SEQUENCE LISTING (55 sequences)

SEQ ID NO: 1 - protein, 29 aa, Artificial sequence
  Synthetic
GCGCGKRTADGSEFESPKKKRKVGSGSGG

SEQ ID NO: 2 - protein, 16 aa, Artificial sequence
  Synthetic
KRPAATKKAGQAKKKK

SEQ ID NO: 3 - protein, 25 aa, Artificial sequence
  Synthetic
MSRRRKANPTKLSENAKKLAKEVEN

SEQ ID NO: 4 - protein, 18 aa, Artificial sequence
  Synthetic
KRTADGSEFESPKKKRKV

SEQ ID NO: 5 - DNA, 6 bp, Artificial sequence
  Synthetic
  misc_feature (1)..(1): n is a, c, g, or t
naaaac

SEQ ID NO: 6 - DNA, 7 bp, Artificial sequence
  Synthetic
  misc_feature (1)..(2): n is a, c, g, or t
  misc_feature (7)..(7): W=A or T
nnagaaw

SEQ ID NO: 7 - DNA, 8 bp, Artificial sequence
  Synthetic
  misc_feature (1)..(4): n is a, c, g, or t
nnnngatt

SEQ ID NO: 8 - DNA, 6 bp, Artificial sequence
  Synthetic
  misc_feature (1)..(2): n is a, c, g, or t
nnagaa

SEQ ID NO: 9 - protein, 844 aa, Artificial sequence
  Synthetic
MGPKKKRKVGSGMATSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYGAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYV

SEQ ID NO: 10 - protein, 842 aa, Artificial sequence
  Synthetic
MATSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYGAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYVGSGPKKKRKV

SEQ ID NO: 11 - protein, 861 aa, Artificial sequence
  Synthetic
MATSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYGGSGSGKRTADGSEFESPKKKRKVGSGSGGAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYV

SEQ ID NO: 12 - protein, 861 aa, Artificial sequence; identical to SEQ ID NO: 11
  Synthetic

SEQ ID NO: 13 - protein, 883 aa, Artificial sequence
  Synthetic
MDYKDHDGDYKDHDIDYKDDDDKATSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYGGSGSGKRTADGSEFESPKKKRKVGSGSGGAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYV

SEQ ID NO: 14 - protein, 930 aa, Artificial sequence
  Synthetic
MATSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYGGSGSGKRTADGSEFESPKKKRKVGSGSGGAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYVGGSGGSGEASGSGRADALDDFDLDMLGSDALDDFDLDMLGSDALDDFDLDMLGSDALDDFDLDMLINSR

SEQ ID NO: 15 - DNA, 4609 bp, Artificial sequence
  Synthetic
aggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcgggagaattcgccaccatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtgagggcccagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaatttataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactaga

SEQ ID NO: 16 - DNA, 8833 bp, Artificial sequence
  Synthetic
caactttgtatagaaaagttggctccgaatttctcgagttaattaaatgtgctgcaaggcgattaagttgggtaacgccagggttttcccagtcacgacgttgtaaaacgacggccagtgaattgtaatacgactcactatagggcgaattggagctaggcctacgtagcgcgcgagctccaccgcggtggactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgaccccaagtttgtacaaaaaagcagcgccaccatggtgagcaagggcgaggagctgttcaccggggtggtgcccatcctggtcgagctggacggcgacgtaaacggccacaagttcagcgtgaggggcgagggcgagggcgatgccaccaacggcaagctgaccctgaagttcatctgcaccaccggcaagctgcccgtgccctggcccaccctcgtgaccaccctgagccacggcgtgcagtgcttcgcccgctaccccgaccacatgaagcagcacgacttcttcaagtccgccatgcccgaaggctacgtccaggagcgcaccatcttcttcaaggacgacggcacctacaagacccgcgccgaggtgaagttcgagggcgacaccctagtgaaccgcatcgagctgaagggcgtcgacttcaaggaggacggcaacatcctggggcacaagctggagtacaacttcaacagccacaacatctatatcatggccgtcaagcagaagaacggcatcaaggtgaacttcaagatccgccacaacgtggaggacggcagcgtgcagctcgccgaccactaccagcagaacacccccatcggcgacggccccgtgctgctgcccgacagccactacctgagcacccagtccgtgctgagcaaagaccccaacgagaagcgcgatcacatggtcctgctggagttccgcaccgccgccgggatcactctcggcatggacgagctgtacaagtaatacccagctttcttgtacaaagtggtacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgacagtactaagcttggtagataagttgatttagccataaaatattgtttccgtgaccccttaattaatagggataacagggtaatctcgaggtttaaacgtcgacgcggccgctactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttattaccctgttatccctaaccggttgcttgcaaaagcagtaattggaaagcactctcaaagaatccaagcttctgacctcttctcttcctcccacagggcctcgagacaattgcatcgatggtaccagtgaaagtctgcaatgaattcgagttggcgcgcctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggagggggctgtccccgtgagctcccaggcgcgcctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcccaggcgcgccaactcggtaccgtatcgatgtcgacgttaacgctagtgatat

SEQ ID NO: 17 - DNA, 4883 bp, Artificial sequence
  Synthetic
cagctggcgaaagggggatgtgctgcaaggcgattaagttgggtaacgccagggttttcccagtcacgacgttgtaaaacgacggccagtgaattgtaatacgactcactatagggcgaattgggccctctagatgcatgctcgagcggccgccagtgtgatggatatctgcagaattcgcccttgcgacgtagggataacagggtaatcctcaggagactgtgtaacaccaatgcaggcaaatcaaccttagtctgaaggcctaggcaaagaagaacacaacttacttcttaaggtcccctccaccccacagtggggcgaggtaggcgtgtacggtgggaggcctatataagcagagctcgtttagtgaaccgtcagatcgcctggagaattcgccaccatggactacaaggatgacgacgataaaacttccggtggcggactgggttccaccgcgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctacaggacggcgagttcatctacaaggtgaagctgcgcggcaccaacttcccctccgacggccccgtaatgcagaagaagacgatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagttggacatcacctcccacaacgaggactacaccatcgtggaacagtacgagcgcgccgagggccgccactccaccggcggcatggacgagctgtacaaggcccgcggttaagatgcatgccagttctaggaattcgagctcggtacccggggatcctctagtcagctgacgcgtgctagcgcggccgcatcgataagcttgtcgacgatatctctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcctcgagcttcctcgctcactgactcgctgcgctcggtcgttcggctgcggcgagcggtatcagctcactcaaaggcggtaataaagggcgaattccagcacactggcggccgttactagtggatccgagctcggtaccaagcttgatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtggccctcctcacgtgctattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgccccggcgtgtcaataatatcactctgtacatccacaaacagacgataacggctctctcttttataggtgtaaaccttaaactgccgtacgtataggctgcgcaactgttgggaagggcgatcggtgcgggcctcttcgctattacgc

SEQ ID NO: 18 - DNA, 10693 bp, Artificial sequence
  Synthetic
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgaaacttaagcttaccggtagccaccatggggcccaagaagaaaaggaaggtcggctccggcatgacacggttcgtccagctgcatctgctgacttcctaccctcccgcaaacctgaaccgggacgatctgggcaatcctaagacagccagactgggaggagtggagcggctgcgggtgagcagccagagcctgaagagggcatggcggacatccgagctgtttcagcagcagctggccggcaccatcggcaccagaacaaagaggctgggcatcgaggtgttcgaggccctgctgggagccggagtgaccgagaagcaggcaagagagtgggcaggacagatcgccaaggtgtatggagccgccaagaaggataacccactggagatcgagcagctggtgcacatcgcccccgaggagagggccagcctggaccagctggtggccacactggccgcagagaagaggggaccaaccgacgaggagctggatgccctgctgcaccaccagacagccgtggacatcgccatgtttggccggatgctggccagcaagacccagttcaatggagaggcagcagtgcaggtggcacacgcaatcggagtgcacgcctccgccatcgaggacgattacttcaccgccgtggacgatctgaacagaaatgatccaggagccgcacacatcggagagtccggctttgcagccgccgtgttctaccagtatatctgcatcgaccgggatctgctgaagagaaacctgggcggcgacgaggtgctgacacagaaggccctgagggccctgaccgaggccgccctgaaggtcggacctagcggcaagcagaacagcttcgccagcagggcctttgcccacttcgccctggccgagaagggcacagatcagcctagaagcctgtccctggccttcgtgaagccagtggccggcaccgactacgcaggcgatgcagtggccgccctgcagcaggtgcgggacaacatggataaggtgtatggcgtgtgcgccgagagcagatgtcagtttaatgtgctgacaggcgagggctccgtggcagacctgctggatttcgtggcagcagaggctagctcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaggcggcgagggaaggggctctctgctgacctgcggcgacgtggaggagaaccctggacctggaccgaaaaagaagcgaaaagttggatccggcatgaagcctcgcaagccacggctgaatgaggcccagcagagatgggtgagggattggtggagggccctgcagccaagggcagagggcgacgagcctatcccaggcgagctgagcgtgatgggaaggggagagcgggcccagctgcggagatgtaccgacgccgatgagctgctgacacagtccgccaccctgctgctggcccacaggctggtggccctgaacggagagaggggaccactgcctgataattctctgagctatgagaggatggcatgggtggcaggcgtgctggccaacgtgaaggacgatctgcgcgatggcaagtccctggccacccacctgggacaggcagcagacgccgagaggccacccatgtctgagctgcgctttcgggcaatgcagaggggcacagcaatgcaggagctgttcctgcactggaggcgcgccctgcagctggccggaggcaagaccgacgtggcacacctggcagacgatctgctgagctggcagatcgagcagggacagagcgccgcacaggcctccaatggcgtgaagtttcactgggcctacgactactatctgtctgccagagatagggccgccgccaaggagcctgagttcaacaaggagatatccaaggggtccggcgaaggaaggggcagcctgctgacatgcggcgatgtggaggagaaccctggacctgctccaaaaaagaaacgcaaggtgggcagcggcatgaccgactacctgctgctgagactgtatggcccactggcctcctggggagagatcgcagtgggagagagcaggcactccgccgtgcagccatccagatctgccctgctgggcctgctgggggccgccctgggcatcgagagacacgacgatgcagcacagcaggccctggtggatggctacaggttcgccatcaagctggagtgtatcggctctcccctgcgcgactatcacaccgtgcaagtgggcgtgccccctaggaagttccagtttagaagccggagacaggagctggccgcagacaaggtggatacaatcctgtctaccagagagtataggtgcgatagcctggccctggtggcagtggaggccctgccaggggcccccgtggacctggccagcctggccgaggccctgcgcaagcctaggtttgccctgtacctgggaaggaagtcctgtccactggccctgccactgtctcccaagatcctggccgcctctagcgtgagagaggtgttcgataacctggagctgccctctctgctgggcctgctggacagataccagccagagcaggcctggccaagcaggcaggatcagcaggccctgaggcctggagtggcaaggtactattgggaggacggcatgacagcaggaatggccccctcctttgaggcacagaggcacgatcagcctctgtctcggcggcggtggcagttcgcaccaagaagggagtgggtggccctgaacgatggaggacagagcggctcgagcgagggaaggggctccctgctgacttgtggcgacgtggaggagaaccctggacctagccctaaaaaaaagcggaaggtaggtaccggcatgagccactatttttccctggtgaggctgatcggctctcccaggcacgacgcatggctgcgggatctgagcagacacggcgaggcctaccgggaccacgcactgatctggagactgttcccaggcgacggagccgcaagggatttcgtgtttcgccggctggaggatgagaagtctttttatgtggtgagcgccagacctccacaggcagacgcaggcctgttccacatccagtctaaggcctacagccctgagctggccgagggcgactgggtgaggttcgatctgcgcgccaacccaacagtgagcgtgagaagggagaatggcagatcccagaggcacgatgtgctgatgcacgccaagcagctggcctccaccgagaagtctgccctgcccgagcggctggaggcagcaggaagagagtggctgaaggacagggcagagcggtggggcctggacctgagaaccgattccctgatgcagaacggctacagacagcagaggctgaagcgcaagggcaagcacatcgccttttctacactggactatcagggcatcgcccaagtgaccgatcctgagcagctgcgccgggccctgctggacggagtgggacactccaagggattcggatgcggcctgctgctggtgaagagggtggatggtaaccaggagggaagaggctccctgctgacatgtggcgacgtggaagaaaaccctggacctggcccgaagaaaaaacgtaaagtgggctccggaatggacctgctgtccgatacatggctgcagtgcaggcacagggacggcaccctgaagcctatcgccatcggccagatcggcctggaggactgtctggagctggtggccccccggcccgacttccggggggccctgtaccagttcctgatcggcctgctgcagacagcctatgcccctgaggacctgcaggagtggagagataggtacgcaaacccaccaaccgcagacgatctggcagaggtgtttgccccctatagggatgccttccagctggagaacagcggccctacattcatgcaggacctgaccctgcccgacgatgtgaatcagctgcctgtgctggagctgctgatcgacgccggcagctcctctaaccagtactttaataagcctgcagtggagcacggaatgtgcgagggctgtttcacacaggccctgctgaccatgcagctgaatgcaccatctggcggaaggggcatcagaacaagcctgcgcggaggaggaccactgaccacactgctggtgcccgcagagcagaacgccaccctgtggcagaagctgtggctgaatgtgctgccactggacgcactggatcaccctccaatcaagatgctgtccgatgtgctgccctggctggcccctaccaggacatctgacgataagcagggccaggacacaccccctgagagcgtgcacccactgcaggcctactggtccatgccaaggcggatcagactggacgcagccaccctggaccagggcgattgcgccgtgtgcggggcccagaacgtgaagcgcatccggcactacagaacaaggcacggcggcaccaattataccggcacatggacccaccctctgaccccatatagcctggattccaagggcgagaagccaccactgagcatcaagggcaggcaggcaggaaggggctaccgcgactggctgggcctggtgctgggaaacgaggaccaccagccagatgcagcacaggtggtgcggcacttcacagcaaagctgggcaagccttccgtgagactgtggtgctttggcttcgacatgtctaatatgaaggccctgtgctggtacgatagcctgctgcctgtgcacggcgtggccccagacgtgcagaggaagtttacccgcagcgtgaagcaggtgctggacagcgccaacgatatggcctccgtgctgcacaagcaggtgaaggcagcatggttcagaaggcctggcgacgcaggacaggagccagcagtgacacagagcttttggcagggcagcgagacagccttctatcaggtgctggagcagctgtctaagctggactttgatagcgccgcagagctggccgcaatctacagggcatggctgcaggcaacacgccggctggtgctgtccctgttcgatcactgggtgctgtctggccccctggaggacatggatatgcagagggtggtgaaggcaagagcagacctggccaaggagctgaataccgggaaagcccagaagccactgtggactattgtgaaccagcatctgaaagagcagggttaacgatagaattctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 19 - DNA, 4796 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaatacgactcactatagggtctagaattcgtgttccccgcacctgcggggatgaaccgtaagttgtgttcttctttgcctaggccttcaggtgttccccgcacctgcggggatgaaccggcggccgctttttttcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 20 - DNA, 4796 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaatacgactcactatagggtctagaattcgtgttccccgcacctgcggggatgaaccgacgaagtggaggaggacacgggcaatgaacttgtgttccccgcacctgcggggatgaaccggcggccgctttttttcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 21 - DNA, 6638 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccctacttccaatccaatgccaccatgggcccgaaaaagaagcgaaaagttggctccggaatgttcctccagaggccaaagccctacagcgacgagtccctggagtctttctttatcagagtggccaataagaacggctatggcgatgtgcacaggttcctggaggccaccaagcggttcctccaggacatcgatcacaacggctaccagacctttccaacagacatcaccaggatcaatccctatagcgccaagaacagctcctctgcccgcacagcctccttcctgaagctggcccagctgacctttaatgagccacctgagctgctgggactggccatcaatcggacaaacatgaagtactccccttctaccagcgccgtggtgagaggagcagaggtgttcccacggtccctgctgagaacccacagcatcccatgctgtcccctgtgcctgagagagaacggctacgccagctatctgtggcacttccagggctacgagtattgtcactcccacaatgtgcctctgatcaccacatgctcttgtggcaaggagtttgactacagggtgagcggcctgaagggcatctgctgtaagtgcaaggagccaatcaccctgacaagccgcgagaatggccacgaggccgcctgtaccgtgtccaactggctggccggccacgagtctaagcctctgccaaacctgcccaagtcctatagatggggactggtgcactggtggatgggcatcaaggactccgagttcgatcacttctcttttgtgcagttctttagcaactggcctcggagcttccactccatcatcgaggacgaggtggagtttaatctggagcacgccgtggtgtccacctctgagctgcggctgaaggatctgctgggcagactgttctttggcagcatcaggctgccagagcgcaatctccagcacaacatcatcctgggcgagctgctgtgctacctggagaaccgcctgtggcaggacaagggcctgatcgccaatctgaagatgaacgccctggaggccacagtgatgctgaattgttccctggatcagatcgcctctatggtggagcagcgcatcctgaagcccaatagaaagtccaagcctaactctccactggacgtgaccgattacctgttccactttggcgacatcttctgcctgtggctggccgagtttcagtccgatgagttcaaccggagcttctacgtgagccggtggtaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 22 - DNA, 7376 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccctacttccaatccaatgccaccatgggcccgaaaaagaagcgaaaagttggctccggaatgcagaccctgaaggagctgatcgccagcaaccccgacgatctgaccacagagctgaagagggccttccgccccctgacacctcacatcgccatcgacggcaatgagctggatgccctgaccatcctggtgaacctgacagacaagaccgacgatcagaaggacctgctggatcgggccaagtgtaagcagaagctgagagatgagaagtggtgggcctcctgcatcaactgcgtgaactacaggcagtctcacaacccaaagttccccgacatccgcagcgagggcgtgatcagaacacaggccctgggcgagctgcccagctttctgctgtctagctccaagatcccaccctaccactggtcttatagccacgactctaagtatgtgaataagagcgccttcctgaccaacgagttttgctgggatggcgagatcagctgtctgggcgagctgctgaaggacgccgatcaccctctgtggaacacactgaagaagctgggctgctcccagaaaacctgtaaggcaatggccaagcagctggccgacatcacactgaccacaatcaatgtgaccctggcccccaactacctgacacagatctctctgcctgactccgatacatcttatatctccctgtctcctgtggccagcctgtccatgcagagccacttccaccagaggctccaggatgagaacaggcactccgccatcacccggttcagccggaccacaaatatgggagtgacagcaatgacctgcggaggagccttcaggatgctgaaaagcggcgccaagttttctagccctccacaccaccggctgaacagcaagagatcctggctgacctccgagcatgtgcagtctctgaagcagtaccagcggctgaataagagcctgatcccagagaactccagaatcgccctgcggagaaagtataagatcgagctgcaaaatatggtgcgctcttggttcgccatgcaggaccacacactggatagcaatatcctgatccagcacctgaaccacgacctgtcctacctgggcgccaccaagcggttcgcctatgatcccgccatgacaaagctgtttaccgagctgctgaagagagagctgtctaacagcatcaacaatggcgagcagcacaccaatggcagctttctggtgctgcctaacatcagagtgtgcggagcaaccgccctgtcctctcctgtgacagtgggcatcccatccctgaccgccttctttggcttcgtgcacgcctttgagaggaatatcaaccgcaccacaagctccttcagggtggagagctttgccatctgcgtgcaccagctgcatgtggagaagcgcggcctgacagccgagttcgtggagaagggcgacggaacaatctccgccccagcaaccagggacgattggcagtgtgatgtggtgttctctctgatcctgaataccaactttgcccagcacatcgaccaggatacactcgtcacctctctgccaaagaggctggcaaggggaagcgccaagatcgccatcgacgatttcaagcacatcaactccttttctacactggaaaccgcaatcgagagcctgccaatcgaggcaggcagatggctgagtctgtacgcccagtccaacaataacctgtctgacctgctggccgccatgacagaggatcaccagctgatggccagctgcgtgggctaccacctgctggaggagcctaaagacaagccaaactccctcagaggctataagcacgccatcgccgagtgtatcatcggcctgatcaattccatcaccttctctagcgagacagatcccaacaccatcttttggagcctgaagaattaccagaactatctggtggtgcagcctcgctccatcaatgacgaaaccaccgataagtcctctctgtaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 23 - DNA, 6512 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccctacttccaatccaatgccaccatgggcccgaaaaagaagcgaaaagttggctccggaatgaagctgccaaccaacctggcctacgagcggagcatcgaccccagcgacgtgtgcttcttcgtggtgtggccagacgataggaaaacccccctgacatacaattcccgcacactgctgggacagatggaggcagccagcctggcatacgacgtgtccggccagcctatcaagtctgccaccgcagaggccctggcacagggcaaccctcaccaggtggatttctgccatgtgccatacggcgccagccacatcgagtgtagcttctccgtgtcttttagctccgagctgcggcagccatataagtgtaattctagcaaggtgaagcagacactggtgcagctggtggagctgtacgaaaccaagatcggctggacagagctggccacccggtatctgatgaatatctgcaacggcaagtggctgtggaagaatacaagaaaggcctactgttggaacatcgtgctgaccccctggccttggaatggcgagaaagtgggcttcgaggacatcaggaccaactatacatcccgccaggacttcaagaacaataagaattggtctgccatcgtggagatgatcaagacagccttctcctctaccgacggcctggccatctttgaggtgagggccacactgcacctgccaaccaacgcaatggtgcgccctagccaggtgttcacagagaaggagagcggctccaagtctaagagcaagacccagaactctagggtgttccagagcaccacaatcgatggagagcggagcccaatcctgggagcctttaagacaggcgccgccatcgccaccatcgacgattggtatccagaggcaaccgagcctctgagggtgggccgctttggagtgcacagggaggacgtgacatgctacagacacccatctaccggcaaggatttctttagcatcctccagcaggccgagcactatatcgaggtgctgagtgccaacaagacacccgcccaggaaaccatcaatgacatgcacttcctgatggccaacctgatcaagggcggcatgtttcagcacaagggcgactaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 24 - DNA, 6053 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccctacttccaatccaatgccaccatgggcccgaaaaagaagcgaaaagttggctccggagtgaagtggtactataagaccatcacattcctgcctgagctgtgcaacaatgagtctctggcagcaaagtgtctgcgggtgctgcacggcttcaattaccagtatgagacaagaaacatcggcgtgtcctttccactgtggtgcgacgccaccgtgggcaagaagatctctttcgtgagcaagaacaagatcgagctggatctgctgctgaagcagcactacttcgtgcagatggagcagctccagtattttcacatcagcaatacagtgctggtgcccgaggactgcacctacgtgagctttcggagatgtcagtccatcgataagctgaccgcagcaggactggcaagaaagatcaggcgcctggagaagcgggccctgtccagaggcgagcagttcgacccatctagctttgcccagaaggagcacacagccatcgcccactaccactctctgggcgagtcctctaagcagaccaatcggaacttcagactgaacatcaggatgctgagtgagcagccaagggagggcaattccatcttcagctcctatggcctgagcaattccgagaactcttttcagcccgtgcctctgatctaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 25 - DNA, 7436 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccctacttccaatccaatgccaccatgggcccgaaaaagaagcgaaaagttggctccggaatgcagaccctgaaggagctgatcgccagcaaccccgacgatctgaccacagagctgaagagggccttccgccccctgacacctcacatcgccatcgacggcaatgagctggatgccctgaccatcctggtgaacctgacagacaagaccgacgatcagaaggacctgctggatcgggccaagtgtaagcagaagctgagagatgagaagtggtgggcctcctgcatcaactgcgtgaactacaggcagtctcacaacccaaagttccccgacatccgcagcgagggcgtgatcagaacacaggccctgggcgagctgcccagctttctgctgtctagctccaagatcccaccctaccactggtcttatagccacgactctaagtatgtgaataagagcgccttcctgaccaacgagttttgctgggatggcgagatcagctgtctgggcgagctgctgaaggacgccgatcaccctctgtggaacacactgaagaagctgggctgctcccagaaaacctgtaaggcaatggccaagcagctggccgacatcacactgaccacaatcaatgtgaccctggcccccaactacctgacacagatctctctgcctgactccgatacatcttatatctccctgtctcctgtggccagcctgtccatgcagagccacttccaccagaggctccaggatgagaacaggcactccgccatcacccggttcagccggaccacaaatatgggagtgacagcaatgacctgcggaggagccttcaggatgctgaaaagcggcgccaagttttctagccctccacaccaccggctgaacagcaagagatcctggctgacctccgagcatgtgcagtctctgaagcagtaccagcggctgaataagagcctgatcccagagaactccagaatcgccctgcggagaaagtataagatcgagctgcaaaatatggtgcgctcttggttcgccatgcaggaccacacactggatagcaatatcctgatccagcacctgaaccacgacctgtcctacctgggcgccaccaagcggttcgcctatgatcccgccatgacaaagctgtttaccgagctgctgaagagagagctgtctaacagcatcaacaatggcgagcagcacaccaatggcagctttctggtgctgcctaacatcagagtgtgcggagcaaccgccctgtcctctcctgtgacagtgggcatcccatccctgaccgccttctttggcttcgtgcacgcctttgagaggaatatcaaccgcaccacaagctccttcagggtggagagctttgccatctgcgtgcaccagctgcatgtggagaagcgcggcctgacagccgagttcgtggagaagggcgacggaacaatctccgccccagcaaccagggacgattggcagtgtgatgtggtgttctctctgatcctgaataccaactttgcccagcacatcgaccaggatacactcgtcacctctctgccaaagaggctggcaaggggaagcgccaagatcgccatcgacgatttcaagcacatcaactccttttctacactggaaaccgcaatcgagagcctgccaatcgaggcaggcagatggctgagtctgtacgcccagtccaacaataacctgtctgacctgctggccgccatgacagaggatcaccagctgatggccagctgcgtgggctaccacctgctggaggagcctaaagacaagccaaactccctcagaggctataagcacgccatcgccgagtgtatcatcggcctgatcaattccatcaccttctctagcgagacagatcccaacaccatcttttggagcctgaagaattaccagaactatctggtggtgcagcctcgctccatcaatgacgaaaccaccgataagtcctctctgggctcgagcgagggaaggggctctctgctgacctgcggcgacgtggaggagaaccctggataattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 26 - DNA, 4801 bp, Artificial sequence
  Synthetic
ttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaatacgactcactatagggtctagaattcctgtgaactgccgagtaggtagctgataacgcgaggtattcggctccgcgacggaggctaaggtgaactgccgagtaggtagctgataacgttgcagcggccgctttttttcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctc

SEQ ID NO: 27 - DNA, 12120 bp, Artificial sequence
  Synthetic
attcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgggtgaactgccgagtaggtagctgataacagtacagcgcggctgaaatcatcattaaagcggtgaactgccgagtaggtagctgataacgaattcgagcgaggaggttacatatgggacctaagaaaaagcgtaaagttggttccggtatgtttttgcaaagacctaaaccttacagcgatgaaagtttagaaagtttctttatccgagtggctaacaaaaatggctacggtgatgtccatcgcttcctagaagccactaaacgattccttcaagacattgaccataatggctatcaaacctttccgactgatataactcggataaacccatactcagctaaaaacagttccagcgcacgaactgcgtcattcctgaaacttgcacaattgacatttaatgaaccgccagagctacttgggttggcaattaacagaacaaacatgaaatactcgccgtcaactagcgcggttgttagaggtgcagaagtctttcctcgcagtttactacggacgcactccatcccctgctgtcctttgtgtctgcgagaaaatggctacgcctcctacctttggcactttcaggggtacgaatactgccacagccataacgtacctttaattaccacttgtagctgtggtaaggagtttgactaccgagtatctgggttaaagggcatttgctgcaaatgcaaggagcctatcaccttaaccagcagggagaacggtcatgaggcagcgtgtactgtttcaaactggcttgctggccatgaatctaaacctctgccaaatcttcctaaaagctaccgatggggtttagttcattggtggatgggtattaaagatagcgagttcgatcacttttcgttcgttcaatttttctcaaactggccaaggtcattccactcgataatcgaagatgaagtagagttcaaccttgagcatgctgttgtcagcacgtctgaattacgactaaaagatcttcttggtcgattgtttttcggttcaattcggttacctgagcggaatcttcaacacaatatcatccttggtgagcttctctgctatttagaaaatcgtttatggcaagacaagggattaatcgccaacctcaaaatgaacgcgttagaggcgactgtaatgttaaattgtagcctcgatcagattgcatcaatggttgaacaacgcatcttgaagccaaatcgaaaaagcaagcccaacagccctcttgatgttaccgattatctatttcatttcggcgatatcttctgtctttggttagctgagttccaaagcgatgagtttaaccgttcgttttatgtgtcgaggtggtaaatgggtcccaagaaaaagcgcaaggtcggtaccggtatgcaaactctgaaagaactaatcgcatccaatcctgacgacttaacaactgaacttaagagagcatttcgtccactcacaccgcatattgcaattgatggtaatgaacttgacgcactgacgatattagtcaatttaaccgataagactgatgatcagaaagacctgctcgatcgagccaaatgcaagcaaaaacttcgagatgaaaaatggtgggctagctgcataaattgcgttaattacagacaaagccataacccaaaattcccggatatacgttctgaaggcgtgattcgaacccaagccctgggtgaattaccgagtttcctactctcgtcctcaaaaatcccaccataccattggtcatatagccatgattcaaaatacgtcaacaaaagcgcattcctcaccaatgagttttgttgggatggtgagatctcatgtttgggtgagcttcttaaagatgcagatcacccactttggaatactttgaaaaagttaggttgttctcaaaaaacttgcaaagcaatggcaaaacaactagctgatattactctcacgactatcaatgtcacactcgcaccaaattacctgactcaaatatctctccccgatagtgatacatcctacatttcactctctcctgttgcatcgctatcgatgcaaagccactttcatcagaggctccaagatgaaaatcggcatagcgcgataacgcggtttagccgaactaccaacatgggggtcactgcgatgacatgtggcggcgcatttagaatgttaaagtctggcgctaagttttccagcccccctcatcaccgattaaacagtaaacgaagttggttgacgtcagagcatgttcagtcattaaaacagtaccagcgcctcaacaaaagcctcatacctgaaaactctcggattgcactccgtagaaaatacaaaatcgagcttcaaaatatggtcagatcttggtttgcaatgcaagaccatacccttgattcgaacatacttatccaacatttgaatcatgacctatcttacttaggagccacaaaacgttttgcatacgatccagcgatgaccaagctctttactgagcttttgaaacgagagttatcaaattcaatcaataatggtgagcaacacactaatggatcgtttttagtcctaccgaatatcagggtttgtggcgcaacagctttaagctccccggtaacggtggggattccatcacttacagctttctttggcttcgttcacgcatttgaacggaatataaatcgcaccacctcatcgtttcgtgttgaatcctttgcgatatgcgtccatcaactacatgtcgagaagcgaggtttgacagcagagtttgtggaaaaaggcgacgggactatatccgctcccgcgacccgggatgactggcagtgtgatgtcgtatttagccttattttgaacaccaactttgctcaacatattgaccaagatacgttagttacatcactaccaaagcgattggctcggggttcagcaaaaattgcgattgatgactttaaacatatcaactcattctcgacattagaaacagcgatcgaatctctgccaatagaagctggtaggtggttatcactttacgcacagtcaaacaataatctaagtgatctattagcagccatgacagaggaccatcagctcatggcaagctgcgtcggttaccacttgttagaggagcccaaagataaaccaaactccctcagaggttacaaacacgctatcgccgagtgcatcattggactcattaactcaatcacctttagctcagagactgatcccaacacaatcttttggtcgctaaagaactatcaaaactacctagtggtacagccaaggagtatcaacgacgaaactaccgacaaatctagcctatgaagcttgaaggaggtaaataatgggcccgaagaaaaagcgaaaagtaggctcgagtatgaaactaccgacaaatctagcctatgagcgctctatcgacccatcagatgtctgtttttttgtcgtctggcccgatgatagaaaaacacctttaacctacaattctcgtactctgctcgggcaaatggaagcggcatcattagcctatgatgtctcaggtcaaccaataaaaagtgccaccgctgaggcgttagctcaagggaaccctcatcaagttgatttctgccacgttccatacggtgcgagtcatattgaatgcagtttctccgtctcgttttcttctgaactacgtcaaccatataagtgtaactcaagcaaagttaaacaaacgctagtgcaattagtcgaactctacgaaacgaaaatcggctggactgagctagcaacccgatatttgatgaacatttgcaacggtaaatggctgtggaaaaatacccgtaaagcctattgctggaacattgtacttacaccttggccttggaacggggaaaaggttggatttgaagatattcgtactaactacacctcacggcaagactttaaaaataataaaaattggtctgctatagttgaaatgatcaaaaccgcattttctagtactgatgggctggcgatatttgaagtcagggccaccttgcacttgccaacgaatgctatggtgcggccaagccaagttttcacagaaaaagaaagtggcagtaaaagtaaatctaaaactcaaaacagtcgagtttttcagagtacaactattgatggtgaacgatcgccaatactaggggcctttaaaacgggagcagctattgcaaccattgacgactggtatcctgaagccactgagccactaagggtcggacggtttggggttcatcgcgaagatgtcacttgctaccgtcatccgtctaccggaaaagattttttctcgatattacaacaagcagagcactatattgaagtgttgagcgccaacaaaactcccgctcaagaaactatcaacgacatgcactttttaatggctaacctgattaagggtgggatgttccagcataaaggagactgactatggggcctaagaaaaagcggaaggtgggttccggagtgaaatggtattataagacaatcacctttctgccagagttgtgcaacaacgagtcactggctgcaaagtgtctccgcgttctgcatggatttaactatcagtatgagacacgaaatatcggcgtttcatttccgctttggtgtgatgcaacggttggaaaaaagatttcatttgtcagcaagaacaagatagaactcgacttactacttaaacaacactatttcgtccaaatggaacaacttcaatactttcatatatccaacactgttctcgtcccagaagattgtacatacgtttcctttagacgctgtcaatctatagataagctcacagcagcagggctggcaaggaaaatcagacgcctggagaaacgtgctctatctagaggcgagcaatttgacccatcatcttttgctcaaaaagagcatactgcaatagcgcactaccactcacttggggagtccagcaaacagacgaaccgcaactttcgactcaatatcaggatgctctcggagcaaccccgtgaggggaactcgatttttagtagctatggcttatcaaattcagaaaactcgtttcagcctgtacccttaatctgagtcgacataaggagatatatcatggcgacaagtttacctacgccctcagcaattacgacttcggcgttagagtatgcattccatactcccgctcgcaatctaacgaaatctcgcggaaaaaacattcatcgttatgtcagtgtaaagatgagtaagaggattacggtagaatctactctagagtgtgatgcctgctatcactttgattttgagccaagtattgttcgcttttgcgcacaaccgattcgatttttatattatctcaatggtcagtctcactcctatgttcctgactttctagttcaatttgataccaacgagtttgttctatatgaagtaaagtcagcttatgctaagaacaaacctgattttgatgttgaatgggaggcgaaagtaaaagcagcaactgaactagggctagaattggagcttgttgaagagagtgatattagggatacggttgtattaaataatcttaagcgcatgcatcgttatgcttcgaaagatgagctgaataacgtacataactctctcttaaaaataataaagtacaatggcgcccaatctgcaagatgcttgggagaacagttgggtttaaaaggccgaactgttttaccaattttgtgcgatttgctgtcaaggtgtttactcgatacacgtttggataagcctctatctcttgaatctcgatttgagttggcctcgtacggcggttcacccaagaaaaagcgcaaggtctaactagtaggaggctcgagatgggaccgaagaaaaagagaaaagtgggatccgcgatggctaagaaagggttctcaagtttccatagaaaagcagtctcttcccaagatacgcttgaatcgatagagcttgtctctagcgctaattgtctagaaagtgttacgtatcaagatatatcagcatttcccgaaacaattgcggtagagattaatttccgattaagcattcttcgctttttagcgcgaaagtgcgaaaccattgtggccaaatcaattgaaccacatcgtgtagagctacagcaaaactatagtagaaaaatacccagtgcaataacgatatatcgatggtggcttgcttttcgaaaatcagactacaaccccattagcttagcacctaatatcaaggatagaggtaatagagagacaaaagtgtcaacagttgttgattctattatggaacaggcagttgaaagagttatatctggacgaaaagtcaatgttagctctgcatataaacgtgttcgacgaaaagttcgtcaatacaatctaactcatggaacgaaatacacgtatcctaagtacgaatctgtaagaaagcgagtaaaaaagaaaaccccatttgagttattagccgcagggaaaggggagagagtagctaagagagagtttcgccgaatgggaaaaaagatcctcacgtctagcgtgctagagagggttgaaatagatcacactgtcgttgacctttttgcagtacacgaagagtatcgaatcccattgggccgaccttggcttactcaattggttgattgttacagtaaagctgtaatcggtttttatttaggtttcgagcctcctagctatgtgtcggtttcccttgcacttaagaatgcaatacaacgcaaagatgacttaatctcctcgtatgaatcgatcgagaatgaatggctatgttatggcatcccagacctactcgtaactgataatggtaaagagtttttgtcgaaagcatttgatcaagcatgtgaatcactattgatcaatgtgcatcaaaataaagttgagacgcccgacaacaaacctcatgttgaacgtaactacgggactattaatacttctctgttagacgatttacctgggaaatccttcagccagtaccttcaaagagaagggtacgactctgtgggagaagctacccttacactcaatgagattagagaaatttacttaatttggttggtggatatttatcataaaaaacccaatcagagaggcactaattgtcctaatgttgcgtggaaaaagggttgtcaagaatgggaaccagaggagttctctggttctaaagacgaattagactttaaatttgctattgttgattacaaacaacttactaaagtagggataactgtctacaaagaactgagttatagcaatgaccgtttagctgaatatagagggaagaaaggaaaccataaagttcagttcaagtataaccctgagtgtatggcagttatttgggtgttggatgaggatatgaatgagtactttacagttaatgcgattgactacgaatatgcaagtagagtatcactttggcaacataaatataacatgaaatatcaagcagaactaaattcagcagaatatgatgaggacaaggaaattgatgcagaaataaaaattgaagaaatcgcagatcgttcaattgttaagactaacaaaatcagagctcggaggcgtggcgctaggcatcaagagaatagcgcaagggctaagtcaatcagtaatgcgaacccggcctcgatacaaaaacatgaagatgaaatcgttagtgcagataatgacgattgggatattgattatgtctgagaaatcgtcaaatgggtcctaagaaaaagaggaaagtaggctcttccatgagtgaaacgcgtgaggctcgtatatcaagagctaaaagggcatttgtatccacaccgagcgttaggaaaatcttgagttacatggatagatgtagagatctatcagacctagagtctgagcctacatgcatgatggtctatggtgcttcgggtgtaggtaaaacgaccgtcatcaagaaatacttaaatcaaaacagaagagagtccgaagccgggggcgatataataccggttttgcatattgagttgccagacaatgcgaagccagtagatgcagcaagggaattgctggttgaaatgggtgacccgctagcactttatgaaactgacttagctagattgacgaaaagactgactgaattaatccctgcggtcggcgtgaagctgattattatcgatgagttccaacatttggtggaagaaaggtcaaatcgggttcttacccaagtaggtaattggctaaaaatgatacttaacaaaacgaaatgtccaattgttatatttggtatgccatactcaaaagttgtactgcaagcaaactcgcaacttcacgggcgattttccattcaggttgaactgcgcccctttagctaccagggaggtagaggtgtatttaaaacttttttggaataccttgataaagccctaccttttgaaaaacaggctggcttagccaacgaaagtttgcagaaaaaattgtatgcattctctcagggaaacatgcgttcgttgagaaaccttatttatcaagcatctatcgaagcaattgataatcagcatgagacgataaccgaagaagatttcgtttttgcatcgaagttgacatcgggcgataaacccaactcatggaaaaatccttttgaggagggtgttgaggtaacagaagatatgttacgaccgccaccaaaagatattggttgggaggactatttgagacattcaaccccgagagtgagtaaaccaggtagaaataaaaactttttcgaataacctaggctgctgcgctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcac

SEQ ID NO: 28 - DNA, 13839 bp, Artificial sequence
  Synthetic
attcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgggtgaactgccgagtaggtagctgataacagtacagcgcggctgaaatcatcattaaagcggtgaactgccgagtaggtagctgataacgaattcgagcgaggaggttacatatgggacctaagaaaaagcgtaaagttggttccggtatgtttttgcaaagacctaaaccttacagcgatgaaagtttagaaagtttctttatccgagtggctaacaaaaatggctacggtgatgtccatcgcttcctagaagccactaaacgattccttcaagacattgaccataatggctatcaaacctttccgactgatataactcggataaacccatactcagctaaaaacagttccagcgcacgaactgcgtcattcctgaaacttgcacaattgacatttaatgaaccgccagagctacttgggttggcaattaacagaacaaacatgaaatactcgccgtcaactagcgcggttgttagaggtgcagaagtctttcctcgcagtttactacggacgcactccatcccctgctgtcctttgtgtctgcgagaaaatggctacgcctcctacctttggcactttcaggggtacgaatactgccacagccataacgtacctttaattaccacttgtagctgtggtaaggagtttgactaccgagtatctgggttaaagggcatttgctgcaaatgcaaggagcctatcaccttaaccagcagggagaacggtcatgaggcagcgtgtactgtttcaaactggcttgctggccatgaatctaaacctctgccaaatcttcctaaaagctaccgatggggtttagttcattggtggatgggtattaaagatagcgagttcgatcacttttcgttcgttcaatttttctcaaactggccaaggtcattccactcgataatcgaagatgaagtagagttcaaccttgagcatgctgttgtcagcacgtctgaattacgactaaaagatcttcttggtcgattgtttttcggttcaattcggttacctgagcggaatcttcaacacaatatcatccttggtgagcttctctgctatttagaaaatcgtttatggcaagacaagggattaatcgccaacctcaaaatgaacgcgttagaggcgactgtaatgttaaattgtagcctcgatcagattgcatcaatggttgaacaacgcatcttgaagccaaatcgaaaaagcaagcccaacagccctcttgatgttaccgattatctatttcatttcggcgatatcttctgtctttggttagctgagttccaaagcgatgagtttaaccgttcgttttatgtgtcgaggtggtaaatgggtcccaagaaaaagcgcaaggtcggtacccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttcgggaggtggttcgggtggctctggatcaagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgtcaggcggtggtagtggtgggagcggtagtgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtggcgcgcctagtggttcagaaacacccggcacatccgagtccgcgacacctgagagtggaggttctggaggttccggcggtaccggtatgcaaactctgaaagaactaatcgcatccaatcctgacgacttaacaactgaacttaagagagcatttcgtccactcacaccgcatattgcaattgatggtaatgaacttgacgcactgacgatattagtcaatttaaccgataagactgatgatcagaaagacctgctcgatcgagccaaatgcaagcaaaaacttcgagatgaaaaatggtgggctagctgcataaattgcgttaattacagacaaagccataacccaaaattcccggatatacgttctgaaggcgtgattcgaacccaagccctgggtgaattaccgagtttcctactctcgtcctcaaaaatcccaccataccattggtcatatagccatgattcaaaatacgtcaacaaaagcgcattcctcaccaatgagttttgttgggatggtgagatctcatgtttgggtgagcttcttaaagatgcagatcacccactttggaatactttgaaaaagttaggttgttctcaaaaaacttgcaaagcaatggcaaaacaactagctgatattactctcacgactatcaatgtcacactcgcaccaaattacctgactcaaatatctctccccgatagtgatacatcctacatttcactctctcctgttgcatcgctatcgatgcaaagccactttcatcagaggctccaagatgaaaatcggcatagcgcgataacgcggtttagccgaactaccaacatgggggtcactgcgatgacatgtggcggcgcatttagaatgttaaagtctggcgctaagttttccagcccccctcatcaccgattaaacagtaaacgaagttggttgacgtcagagcatgttcagtcattaaaacagtaccagcgcctcaacaaaagcctcatacctgaaaactctcggattgcactccgtagaaaatacaaaatcgagcttcaaaatatggtcagatcttggtttgcaatgcaagaccatacccttgattcgaacatacttatccaacatttgaatcatgacctatcttacttaggagccacaaaacgttttgcatacgatccagcgatgaccaagctctttactgagcttttgaaacgagagttatcaaattcaatcaataatggtgagcaacacactaatggatcgtttttagtcctaccgaatatcagggtttgtggcgcaacagctttaagctccccggtaacggtggggattccatcacttacagctttctttggcttcgttcacgcatttgaacggaatataaatcgcaccacctcatcgtttcgtgttgaatcctttgcgatatgcgtccatcaactacatgtcgagaagcgaggtttgacagcagagtttgtggaaaaaggcgacgggactatatccgctcccgcgacccgggatgactggcagtgtgatgtcgtatttagccttattttgaacaccaactttgctcaacatattgaccaagatacgttagttacatcactaccaaagcgattggctcggggttcagcaaaaattgcgattgatgactttaaacatatcaactcattctcgacattagaaacagcgatcgaatctctgccaatagaagctggtaggtggttatcactttacgcacagtcaaacaataatctaagtgatctattagcagccatgacagaggaccatcagctcatggcaagctgcgtcggttaccacttgttagaggagcccaaagataaaccaaactccctcagaggttacaaacacgctatcgccgagtgcatcattggactcattaactcaatcacctttagctcagagactgatcccaacacaatcttttggtcgctaaagaactatcaaaactacctagtggtacagccaaggagtatcaacgacgaaactaccgacaaatctagcctatgaagcttgaaggaggtaaataatgggcccgaagaaaaagcgaaaagtaggctcgagtatgaaactaccgacaaatctagcctatgagcgctctatcgacccatcagatgtctgtttttttgtcgtctggcccgatgatagaaaaacacctttaacctacaattctcgtactctgctcgggcaaatggaagcggcatcattagcctatgatgtctcaggtcaaccaataaaaagtgccaccgctgaggcgttagctcaagggaaccctcatcaagttgatttctgccacgttccatacggtgcgagtcatattgaatgcagtttctccgtctcgttttcttctgaactacgtcaaccatataagtgtaactcaagcaaagttaaacaaacgctagtgcaattagtcgaactctacgaaacgaaaatcggctggactgagctagcaacccgatatttgatgaacatttgcaacggtaaatggctgtggaaaaatacccgtaaagcctattgctggaacattgtacttacaccttggccttggaacggggaaaaggttggatttgaagatattcgtactaactacacctcacggcaagactttaaaaataataaaaattggtctgctatagttgaaatgatcaaaaccgcattttctagtactgatgggctggcgatatttgaagtcagggccaccttgcacttgccaacgaatgctatggtgcggccaagccaagttttcacagaaaaagaaagtggcagtaaaagtaaatctaaaactcaaaacagtcgagtttttcagagtacaactattgatggtgaacgatcgccaatactaggggcctttaaaacgggagcagctattgcaaccattgacgactggtatcctgaagccactgagccactaagggtcggacggtttggggttcatcgcgaagatgtcacttgctaccgtcatccgtctaccggaaaagattttttctcgatattacaacaagcagagcactatattgaagtgttgagcgccaacaaaactcccgctcaagaaactatcaacgacatgcactttttaatggctaacctgattaagggtgggatgttccagcataaaggagactgactatggggcctaagaaaaagcggaaggtgggttccggagtgaaatggtattataagacaatcacctttctgccagagttgtgcaacaacgagtcactggctgcaaagtgtctccgcgttctgcatggatttaactatcagtatgagacacgaaatatcggcgtttcatttccgctttggtgtgatgcaacggttggaaaaaagatttcatttgtcagcaagaacaagatagaactcgacttactacttaaacaacactatttcgtccaaatggaacaacttcaatactttcatatatccaacactgttctcgtcccagaagattgtacatacgtttcctttagacgctgtcaatctatagataagctcacagcagcagggctggcaaggaaaatcagacgcctggagaaacgtgctctatctagaggcgagcaatttgacccatcatcttttgctcaaaaagagcatactgcaatagcgcactaccactcacttggggagtccagcaaacagacgaaccgcaactttcgactcaatatcaggatgctctcggagcaaccccgtgaggggaactcgatttttagtagctatggcttatcaaattcagaaaactcgtttcagcctgtacccttaatctgagtcgacataaggagatatatcatggcgacaagtttacctacgccctcagcaattacgacttcggcgttagagtatgcattccatactcccgctcgcaatctaacgaaatctcgcggaaaaaacattcatcgttatgtcagtgtaaagatgagtaagaggattacggtagaatctactctagagtgtgatgcctgctatcactttgattttgagccaagtattgttcgcttttgcgcacaaccgattcgatttttatattatctcaatggtcagtctcactcctatgttcctgactttctagttcaatttgataccaacgagtttgttctatatgaagtaaagtcagcttatgctaagaacaaacctgattttgatgttgaatgggaggcgaaagtaaaagcagcaactgaactagggctagaattggagcttgttgaagagagtgatattagggatacggttgtattaaataatcttaagcgcatgcatcgttatgcttcgaaagatgagctgaataacgtacataactctctcttaaaaataataaagtacaatggcgcccaatctgcaagatgcttgggagaacagttgggtttaaaaggccgaactgttttaccaattttgtgcgatttgctgtcaaggtgtttactcgatacacgtttggataagcctctatctcttgaatctcgatttgagttggcctcgtacggcggttcacccaagaaaaagcgcaaggtctaactagtaggaggctcgagatgggaccgaagaaaaagagaaaagtgggatccgcgatggctaagaaagggttctcaagtttccatagaaaagcagtctcttcccaagatacgcttgaatcgatagagcttgtctctagcgctaattgtctagaaagtgttacgtatcaagatatatcagcatttcccgaaacaattgcggtagagattaatttccgattaagcattcttcgctttttagcgcgaaagtgcgaaaccattgtggccaaatcaattgaaccacatcgtgtagagctacagcaaaactatagtagaaaaatacccagtgcaataacgatatatcgatggtggcttgcttttcgaaaatcagactacaaccccattagcttagcacctaatatcaaggatagaggtaatagagagacaaaagtgtcaacagttgttgattctattatggaacaggcagttgaaagagttatatctggacgaaaagtcaatgttagctctgcatataaacgtgttcgacgaaaagttcgtcaatacaatctaactcatggaacgaaatacacgtatcctaagtacgaatctgtaagaaagcgagtaaaaaagaaaaccccatttgagttattagccgcagggaaaggggagagagtagctaagagagagtttcgccgaatgggaaaaaagatcctcacgtctagcgtgctagagagggttgaaatagatcacactgtcgttgacctttttgcagtacacgaagagtatcgaatcccattgggccgaccttggcttactcaattggttgattgttacagtaaagctgtaatcggtttttatttaggtttcgagcctcctagctatgtgtcggtttcccttgcacttaagaatgcaatacaacgcaaagatgacttaatctcctcgtatgaatcgatcgagaatgaatggctatgttatggcatcccagacctactcgtaactgataatggtaaagagtttttgtcgaaagcatttgatcaagcatgtgaatcactattgatcaatgtgcatcaaaataaagttgagacgcccgacaacaaacctcatgttgaacgtaactacgggactattaatacttctctgttagacgatttacctgggaaatccttcagccagtaccttcaaagagaagggtacgactctgtgggagaagctacccttacactcaatgagattagagaaatttacttaatttggttggtggatatttatcataaaaaacccaatcagagaggcactaattgtcctaatgttgcgtggaaaaagggttgtcaagaatgggaaccagaggagttctctggttctaaagacgaattagactttaaatttgctattgttgattacaaacaacttactaaagtagggataactgtctacaaagaactgagttatagcaatgaccgtttagctgaatatagagggaagaaaggaaaccataaagttcagttcaagtataaccctgagtgtatggcagttatttgggtgttggatgaggatatgaatgagtactttacagttaatgcgattgactacgaatatgcaagtagagtatcactttggcaacataaatataacatgaaatatcaagcagaactaaattcagcagaatatgatgaggacaaggaaattgatgcagaaataaaaattgaagaaatcgcagatcgttcaattgttaagactaacaaaatcagagctcggaggcgtggcgctaggcatcaagagaatagcgcaagggctaagtcaatcagtaatgcgaacccggcctcgatacaaaaacatgaagatgaaatcgttagtgcagataatgacgattgggatattgattatgtctgagaaatcgtcaaatgggtcctaagaaaaagaggaaagtaggctcttccatgagtgaaacgcgtgaggctcgtatatcaagagctaaaagggcatttgtatccacaccgagcgttaggaaaatcttgagttacatggatagatgtagagatctatcagacctagagtctgagcctacatgcatgatggtctatggtgcttcgggtgtaggtaaaacgaccgtcatcaagaaatacttaaatcaaaacagaagagagtccgaagccgggggcgatataataccggttttgcatattgagttgccagacaatgcgaagccagtagatgcagcaagggaattgctggttgaaatgggtgacccgctagcactttatgaaactgacttagctagattgacgaaaagactgactgaattaatccctgcggtcggcgtgaagctgattattatcgatgagttccaacatttggtggaagaaaggtcaaatcgggttcttacccaagtaggtaattggctaaaaatgatacttaacaaaacgaaatgtccaattgttatatttggtatgccatactcaaaagttgtactgcaagcaaactcgcaacttcacgggcgattttccattcaggttgaactgcgcccctttagctaccagggaggtagaggtgtatttaaaacttttttggaataccttgataaagccctaccttttgaaaaacaggctggcttagccaacgaaagtttgcagaaaaaattgtatgcattctctcagggaaacatgcgttcgttgagaaaccttatttatcaagcatctatcgaagcaattgataatcagcatgagacgataaccgaagaagatttcgtttttgcatcgaagttgacatcgggcgataaacccaactcatggaaaaatccttttgaggagggtgttgaggtaacagaagatatgttacgaccgccaccaaaagatattggttgggaggactatttgagacattcaaccccgagagtgagtaaaccaggtagaaataaaaactttttcgaataacctaggctgctgcgctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcac

SEQ ID NO: 29 - DNA, 12372 bp, Artificial sequence
  Synthetic
attcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgggtgaactgccgagtaggtagctgataacagtacagcgcggctgaaatcatcattaaagcggtgaactgccgagtaggtagctgataacgaattcgagcgaggaggttacatatgggacctaagaaaaagcgtaaagttggttccggtatgtttttgcaaagacctaaaccttacagcgatgaaagtttagaaagtttctttatccgagtggctaacaaaaatggctacggtgatgtccatcgcttcctagaagccactaaacgattccttcaagacattgaccataatggctatcaaacctttccgactgatataactcggataaacccatactcagctaaaaacagttccagcgcacgaactgcgtcattcctgaaacttgcacaattgacatttaatgaaccgccagagctacttgggttggcaattaacagaacaaacatgaaatactcgccgtcaactagcgcggttgttagaggtgcagaagtctttcctcgcagtttactacggacgcactccatcccctgctgtcctttgtgtctgcgagaaaatggctacgcctcctacctttggcactttcaggggtacgaatactgccacagccataacgtacctttaattaccacttgtagctgtggtaaggagtttgactaccgagtatctgggttaaagggcatttgctgcaaatgcaaggagcctatcaccttaaccagcagggagaacggtcatgaggcagcgtgtactgtttcaaactggcttgctggccatgaatctaaacctctgccaaatcttcctaaaagctaccgatggggtttagttcattggtggatgggtattaaagatagcgagttcgatcacttttcgttcgttcaatttttctcaaactggccaaggtcattccactcgataatcgaagatgaagtagagttcaaccttgagcatgctgttgtcagcacgtctgaattacgactaaaagatcttcttggtcgattgtttttcggttcaattcggttacctgagcggaatcttcaacacaatatcatccttggtgagcttctctgctatttagaaaatcgtttatggcaagacaagggattaatcgccaacctcaaaatgaacgcgttagaggcgactgtaatgttaaattgtagcctcgatcagattgcatcaatggttgaacaacgcatcttgaagccaaatcgaaaaagcaagcccaacagccctcttgatgttaccgattatctatttcatttcggcgatatcttctgtctttggttagctgagttccaaagcgatgagtttaaccgttcgttttatgtgtcgaggtggtaaatgggtcccaagaaaaagcgcaaggtcggtaccggtatgcaaactctgaaagaactaatcgcatccaatcctgacgacttaacaactgaacttaagagagcatttcgtccactcacaccgcatattgcaattgatggtaatgaacttgacgcactgacgatattagtcaatttaaccgataagactgatgatcagaaagacctgctcgatcgagccaaatgcaagcaaaaacttcgagatgaaaaatggtgggctagctgcataaattgcgttaattacagacaaagccataacccaaaattcccggatatacgttctgaaggcgtgattcgaacccaagccctgggtgaattaccgagtttcctactctcgtcctcaaaaatcccaccataccattggtcatatagccatgattcaaaatacgtcaacaaaagcgcattcctcaccaatgagttttgttgggatggtgagatctcatgtttgggtgagcttcttaaagatgcagatcacccactttggaatactttgaaaaagttaggttgttctcaaaaaacttgcaaagcaatggcaaaacaactagctgatattactctcacgactatcaatgtcacactcgcaccaaattacctgactcaaatatctctccccgatagtgatacatcctacatttcactctctcctgttgcatcgctatcgatgcaaagccactttcatcagaggctccaagatgaaaatcggcatagcgcgataacgcggtttagccgaactaccaacatgggggtcactgcgatgacatgtggcggcgcatttagaatgttaaagtctggcgctaagttttccagcccccctcatcaccgattaaacagtaaacgaagttggttgacgtcagagcatgttcagtcattaaaacagtaccagcgcctcaacaaaagcctcatacctgaaaactctcggattgcactccgtagaaaatacaaaatcgagcttcaaaatatggtcagatcttggtttgcaatgcaagaccatacccttgattcgaacatacttatccaacatttgaatcatgacctatcttacttaggagccacaaaacgttttgcatacgatccagcgatgaccaagctctttactgagcttttgaaacgagagttatcaaattcaatcaataatggtgagcaacacactaatggatcgtttttagtcctaccgaatatcagggtttgtggcgcaacagctttaagctccccggtaacggtggggattccatcacttacagctttctttggcttcgttcacgcatttgaacggaatataaatcgcaccacctcatcgtttcgtgttgaatcctttgcgatatgcgtccatcaactacatgtcgagaagcgaggtttgacagcagagtttgtggaaaaaggcgacgggactatatccgctcccgcgacccgggatgactggcagtgtgatgtcgtatttagccttattttgaacaccaactttgctcaacatattgaccaagatacgttagttacatcactaccaaagcgattggctcggggttcagcaaaaattgcgattgatgactttaaacatatcaactcattctcgacattagaaacagcgatcgaatctctgccaatagaagctggtaggtggttatcactttacgcacagtcaaacaataatctaagtgatctattagcagccatgacagaggaccatcagctcatggcaagctgcgtcggttaccacttgttagaggagcccaaagataaaccaaactccctcagaggttacaaacacgctatcgccgagtgcatcattggactcattaactcaatcacctttagctcagagactgatcccaacacaatcttttggtcgctaaagaactatcaaaactacctagtggtacagccaaggagtatcaacgacgaaactaccgacaaatctagcctatgaagcttgaaggaggtaaataatgggcccgaagaaaaagcgaaaagtaggcgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaggcgcgcctagtggttcagaaacacccggcacatccgagtccgcgacacctgagagtggaggttctgggagtatgaaactaccgacaaatctagcctatgagcgctctatcgacccatcagatgtctgtttttttgtcgtctggcccgatgatagaaaaacacctttaacctacaattctcgtactctgctcgggcaaatggaagcggcatcattagcctatgatgtctcaggtcaaccaataaaaagtgccaccgctgaggcgttagctcaagggaaccctcatcaagttgatttctgccacgttccatacggtgcgagtcatattgaatgcagtttctccgtctcgttttcttctgaactacgtcaaccatataagtgtaactcaagcaaagttaaacaaacgctagtgcaattagtcgaactctacgaaacgaaaatcggctggactgagctagcaacccgatatttgatgaacatttgcaacggtaaatggctgtggaaaaatacccgtaaagcctattgctggaacattgtacttacaccttggccttggaacggggaaaaggttggatttgaagatattcgtactaactacacctcacggcaagactttaaaaataataaaaattggtctgctatagttgaaatgatcaaaaccgcattttctagtactgatgggctggcgatatttgaagtcagggccaccttgcacttgccaacgaatgctatggtgcggccaagccaagttttcacagaaaaagaaagtggcagtaaaagtaaatctaaaactcaaaacagtcgagtttttcagagtacaactattgatggtgaacgatcgccaatactaggggcctttaaaacgggagcagctattgcaaccattgacgactggtatcctgaagccactgagccactaagggtcggacggtttggggttcatcgcgaagatgtcacttgctaccgtcatccgtctaccggaaaagattttttctcgatattacaacaagcagagcactatattgaagtgttgagcgccaacaaaactcccgctcaagaaactatcaacgacatgcactttttaatggctaacctgattaagggtgggatgttccagcataaaggagactgactatggggcctaagaaaaagcggaaggtgggttccggagtgaaatggtattataagacaatcacctttctgccagagttgtgcaacaacgagtcactggctgcaaagtgtctccgcgttctgcatggatttaactatcagtatgagacacgaaatatcggcgtttcatttccgctttggtgtgatgcaacggttggaaaaaagatttcatttgtcagcaagaacaagatagaactcgacttactacttaaacaacactatttcgtccaaatggaacaacttcaatactttcatatatccaacactgttctcgtcccagaagattgtacatacgtttcctttagacgctgtcaatctatagataagctcacagcagcagggctggcaaggaaaatcagacgcctggagaaacgtgctctatctagaggcgagcaatttgacccatcatcttttgctcaaaaagagcatactgcaatagcgcactaccactcacttggggagtccagcaaacagacgaaccgcaactttcgactcaatatcaggatgctctcggagcaaccccgtgaggggaactcgatttttagtagctatggcttatcaaattcagaaaactcgtttcagcctgtacccttaatctgagtcgacataaggagatatatcatggcgacaagtttacctacgccctcagcaattacgacttcggcgttagagtatgcattccatactcccgctcgcaatctaacgaaatctcgcggaaaaaacattcatcgttatgtcagtgtaaagatgagtaagaggattacggtagaatctactctagagtgtgatgcctgctatcactttgattttgagccaagtattgttcgcttttgcgcacaaccgattcgatttttatattatctcaatggtcagtctcactcctatgttcctgactttctagttcaatttgataccaacgagtttgttctatatgaagtaaagtcagcttatgctaagaacaaacctgattttgatgttgaatgggaggcgaaagtaaaagcagcaactgaactagggctagaattggagcttgttgaagagagtgatattagggatacggttgtattaaataatcttaagcgcatgcatcgttatgcttcgaaagatgagctgaataacgtacataactctctcttaaaaataataaagtacaatggcgcccaatctgcaagatgcttgggagaacagttgggtttaaaaggccgaactgttttaccaattttgtgcgatttgctgtcaaggtgtttactcgatacacgtttggataagcctctatctcttgaatctcgatttgagttggcctcgtacggcggttcacccaagaaaaagcgcaaggtctaactagtaggaggctcgagatgggaccgaagaaaaagagaaaagtgggatccgcgatggctaagaaagggttctcaagtttccatagaaaagcagtctcttcccaagatacgcttgaatcgatagagcttgtctctagcgctaattgtctagaaagtgttacgtatcaagatatatcagcatttcccgaaacaattgcggtagagattaatttccgattaagcattcttcgctttttagcgcgaaagtgcgaaaccattgtggccaaatcaattgaaccacatcgtgtagagctacagcaaaactatagtagaaaaatacccagtgcaataacgatatatcgatggtggcttgcttttcgaaaatcagactacaaccccattagcttagcacctaatatcaaggatagaggtaatagagagacaaaagtgtcaacagttgttgattctattatggaacaggcagttgaaagagttatatctggacgaaaagtcaatgttagctctgcatataaacgtgttcgacgaaaagttcgtcaatacaatctaactcatggaacgaaatacacgtatcctaagtacgaatctgtaagaaagcgagtaaaaaagaaaaccccatttgagttattagccgcagggaaaggggagagagtagctaagagagagtttcgccgaatgggaaaaaagatcctcacgtctagcgtgctagagagggttgaaatagatcacactgtcgttgacctttttgcagtacacgaagagtatcgaatcccattgggccgaccttggcttactcaattggttgattgttacagtaaagctgtaatcggtttttatttaggtttcgagcctcctagctatgtgtcggtttcccttgcacttaagaatgcaatacaacgcaaagatgacttaatctcctcgtatgaatcgatcgagaatgaatggctatgttatggcatcccagacctactcgtaactgataatggtaaagagtttttgtcgaaagcatttgatcaagcatgtgaatcactattgatcaatgtgcatcaaaataaagttgagacgcccgacaacaaacctcatgttgaacgtaactacgggactattaatacttctctgttagacgatttacctgggaaatccttcagccagtaccttcaaagagaagggtacgactctgtgggagaagctacccttacactcaatgagattagagaaatttacttaatttggttggtggatatttatcataaaaaacccaatcagagaggcactaattgtcctaatgttgcgtggaaaaagggttgtcaagaatgggaaccagaggagttctctggttctaaagacgaattagactttaaatttgctattgttgattacaaacaacttactaaagtagggataactgtctacaaagaactgagttatagcaatgaccgtttagctgaatatagagggaagaaaggaaaccataaagttcagttcaagtataaccctgagtgtatggcagttatttgggtgttggatgaggatatgaatgagtactttacagttaatgcgattgactacgaatatgcaagtagagtatcactttggcaacataaatataacatgaaatatcaagcagaactaaattcagcagaatatgatgaggacaaggaaattgatgcagaaataaaaattgaagaaatcgcagatcgttcaattgttaagactaacaaaatcagagctcggaggcgtggcgctaggcatcaagagaatagcgcaagggctaagtcaatcagtaatgcgaacccggcctcgatacaaaaacatgaagatgaaatcgttagtgcagataatgacgattgggatattgattatgtctgagaaatcgtcaaatgggtcctaagaaaaagaggaaagtaggctcttccatgagtgaaacgcgtgaggctcgtatatcaagagctaaaagggcatttgtatccacaccgagcgttaggaaaatcttgagttacatggatagatgtagagatctatcagacctagagtctgagcctacatgcatgatggtctatggtgcttcgggtgtaggtaaaacgaccgtcatcaagaaatacttaaatcaaaacagaagagagtccgaagccgggggcgatataataccggttttgcatattgagttgccagacaatgcgaagccagtagatgcagcaagggaattgctggttgaaatgggtgacccgctagcactttatgaaactgacttagctagattgacgaaaagactgactgaattaatccctgcggtcggcgtgaagctgattattatcgatgagttccaacatttggtggaagaaaggtcaaatcgggttcttacccaagtaggtaattggctaaaaatgatacttaacaaaacgaaatgtccaattgttatatttggtatgccatactcaaaagttgtactgcaagcaaactcgcaacttcacgggcgattttccattcaggttgaactgcgcccctttagctaccagggaggtagaggtgtatttaaaacttttttggaataccttgataaagccctaccttttgaaaaacaggctggcttagccaacgaaagtttgcagaaaaaattgtatgcattctctcagggaaacatgcgttcgttgagaaaccttatttatcaagcatctatcgaagcaattgataatcagcatgagacgataaccgaagaagatttcgtttttgcatcgaagttgacatcgggcgataaacccaactcatggaaaaatccttttgaggagggtgttgaggtaacagaagatatgttacgaccgccaccaaaagatattggttgggaggactatttgagacattcaaccccgagagtgagtaaaccaggtagaaataaaaactttttcgaataacctaggctgctgcgctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcac

SEQ ID NO: 30 - DNA, 13833 bp, Artificial sequence
  Synthetic
attcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgggtgaactgccgagtaggtagctgataacagtacagcgcggctgaaatcatcattaaagcggtgaactgccgagtaggtagctgataacgaattcgagcgaggaggttacatatgggacctaagaaaaagcgtaaagttggttccggtatgtttttgcaaagacctaaaccttacagcgatgaaagtttagaaagtttctttatccgagtggctaacaaaaatggctacggtgatgtccatcgcttcctagaagccactaaacgattccttcaagacattgaccataatggctatcaaacctttccgactgatataactcggataaacccatactcagctaaaaacagttccagcgcacgaactgcgtcattcctgaaacttgcacaattgacatttaatgaaccgccagagctacttgggttggcaattaacagaacaaacatgaaatactcgccgtcaactagcgcggttgttagaggtgcagaagtctttcctcgcagtttactacggacgcactccatcccctgctgtcctttgtgtctgcgagaaaatggctacgcctcctacctttggcactttcaggggtacgaatactgccacagccataacgtacctttaattaccacttgtagctgtggtaaggagtttgactaccgagtatctgggttaaagggcatttgctgcaaatgcaaggagcctatcaccttaaccagcagggagaacggtcatgaggcagcgtgtactgtttcaaactggcttgctggccatgaatctaaacctctgccaaatcttcctaaaagctaccgatggggtttagttcattggtggatgggtattaaagatagcgagttcgatcacttttcgttcgttcaatttttctcaaactggccaaggtcattccactcgataatcgaagatgaagtagagttcaaccttgagcatgctgttgtcagcacgtctgaattacgactaaaagatcttcttggtcgattgtttttcggttcaattcggttacctgagcggaatcttcaacacaatatcatccttggtgagcttctctgctatttagaaaatcgtttatggcaagacaagggattaatcgccaacctcaaaatgaacgcgttagaggcgactgtaatgttaaattgtagcctcgatcagattgcatcaatggttgaacaacgcatcttgaagccaaatcgaaaaagcaagcccaacagccctcttgatgttaccgattatctatttcatttcggcgatatcttctgtctttggttagctgagttccaaagcgatgagtttaaccgttcgttttatgtgtcgaggtggtaaatgggtcccaagaaaaagcgcaaggtcggtaccggtatgcaaactctgaaagaactaatcgcatccaatcctgacgacttaacaactgaacttaagagagcatttcgtccactcacaccgcatattgcaattgatggtaatgaacttgacgcactgacgatattagtcaatttaaccgataagactgatgatcagaaagacctgctcgatcgagccaaatgcaagcaaaaacttcgagatgaaaaatggtgggctagctgcataaattgcgttaattacagacaaagccataacccaaaattcccggatatacgttctgaaggcgtgattcgaacccaagccctgggtgaattaccgagtttcctactctcgtcctcaaaaatcccaccataccattggtcatatagccatgattcaaaatacgtcaacaaaagcgcattcctcaccaatgagttttgttgggatggtgagatctcatgtttgggtgagcttcttaaagatgcagatcacccactttggaatactttgaaaaagttaggttgttctcaaaaaacttgcaaagcaatggcaaaacaactagctgatattactctcacgactatcaatgtcacactcgcaccaaattacctgactcaaatatctctccccgatagtgatacatcctacatttcactctctcctgttgcatcgctatcgatgcaaagccactttcatcagaggctccaagatgaaaatcggcatagcgcgataacgcggtttagccgaactaccaacatgggggtcactgcgatgacatgtggcggcgcatttagaatgttaaagtctggcgctaagttttccagcccccctcatcaccgattaaacagtaaacgaagttggttgacgtcagagcatgttcagtcattaaaacagtaccagcgcctcaacaaaagcctcatacctgaaaactctcggattgcactccgtagaaaatacaaaatcgagcttcaaaatatggtcagatcttggtttgcaatgcaagaccatacccttgattcgaacatacttatccaacatttgaatcatgacctatcttacttaggagccacaaaacgttttgcatacgatccagcgatgaccaagctctttactgagcttttgaaacgagagttatcaaattcaatcaataatggtgagcaacacactaatggatcgtttttagtcctaccgaatatcagggtttgtggcgcaacagctttaagctccccggtaacggtggggattccatcacttacagctttctttggcttcgttcacgcatttgaacggaatataaatcgcaccacctcatcgtttcgtgttgaatcctttgcgatatgcgtccatcaactacatgtcgagaagcgaggtttgacagcagagtttgtggaaaaaggcgacgggactatatccgctcccgcgacccgggatgactggcagtgtgatgtcgtatttagccttattttgaacaccaactttgctcaacatattgaccaagatacgttagttacatcactaccaaagcgattggctcggggttcagcaaaaattgcgattgatgactttaaacatatcaactcattctcgacattagaaacagcgatcgaatctctgccaatagaagctggtaggtggttatcactttacgcacagtcaaacaataatctaagtgatctattagcagccatgacagaggaccatcagctcatggcaagctgcgtcggttaccacttgttagaggagcccaaagataaaccaaactccctcagaggttacaaacacgctatcgccgagtgcatcattggactcattaactcaatcacctttagctcagagactgatcccaacacaatcttttggtcgctaaagaactatcaaaactacctagtggtacagccaaggagtatcaacgacgaaactaccgacaaatctagcctatgaagcttgaaggaggtaaataatgggcccgaagaaaaagcgaaaagtaggccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttcgggaggtggttcgggtggctctggatcaagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgtcaggcggtggtagtggtgggagcggtagtgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtggcgcgcctagtggttcagaaacacccggcacatccgagtccgcgacacctgagagtggaggttctggaggttccggctcgagtatgaaactaccgacaaatctagcctatgagcgctctatcgacccatcagatgtctgtttttttgtcgtctggcccgatgatagaaaaacacctttaacctacaattctcgtactctgctcgggcaaatggaagcggcatcattagcctatgatgtctcaggtcaaccaataaaaagtgccaccgctgaggcgttagctcaagggaaccctcatcaagttgatttctgccacgttccatacggtgcgagtcatattgaatgcagtttctccgtctcgttttcttctgaactacgtcaaccatataagtgtaactcaagcaaagttaaacaaacgctagtgcaattagtcgaactctacgaaacgaaaatcggctggactgagctagcaacccgatatttgatgaacatttgcaacggtaaatggctgtggaaaaatacccgtaaagcctattgctggaacattgtacttacaccttggccttggaacggggaaaaggttggatttgaagatattcgtactaactacacctcacggcaagactttaaaaataataaaaattggtctgctatagttgaaatgatcaaaaccgcattttctagtactgatgggctggcgatatttgaagtcagggccaccttgcacttgccaacgaatgctatggtgcggccaagccaagttttcacagaaaaagaaagtggcagtaaaagtaaatctaaaactcaaaacagtcgagtttttcagagtacaactattgatggtgaacgatcgccaatactaggggcctttaaaacgggagcagctattgcaaccattgacgactggtatcctgaagccactgagccactaagggtcggacggtttggggttcatcgcgaagatgtcacttgctaccgtcatccgtctaccggaaaagattttttctcgatattacaacaagcagagcactatattgaagtgttgagcgccaacaaaactcccgctcaagaaactatcaacgacatgcactttttaatggctaacctgattaagggtgggatgttccagcataaaggagactgactatggggcctaagaaaaagcggaaggtgggttccggagtgaaatggtattataagacaatcacctttctgccagagttgtgcaacaacgagtcactggctgcaaagtgtctccgcgttctgcatggatttaactatcagtatgagacacgaaatatcggcgtttcatttccgctttggtgtgatgcaacggttggaaaaaagatttcatttgtcagcaagaacaagatagaactcgacttactacttaaacaacactatttcgtccaaatggaacaacttcaatactttcatatatccaacactgttctcgtcccagaagattgtacatacgtttcctttagacgctgtcaatctatagataagctcacagcagcagggctggcaaggaaaatcagacgcctggagaaacgtgctctatctagaggcgagcaatttgacccatcatcttttgctcaaaaagagcatactgcaatagcgcactaccactcacttggggagtccagcaaacagacgaaccgcaactttcgactcaatatcaggatgctctcggagcaaccccgtgaggggaactcgatttttagtagctatggcttatcaaattcagaaaactcgtttcagcctgtacccttaatctgagtcgacataaggagatatatcatggcgacaagtttacctacgccctcagcaattacgacttcggcgttagagtatgcattccatactcccgctcgcaatctaacgaaatctcgcggaaaaaacattcatcgttatgtcagtgtaaagatgagtaagaggattacggtagaatctactctagagtgtgatgcctgctatcactttgattttgagccaagtattgttcgcttttgcgcacaaccgattcgatttttatattatctcaatggtcagtctcactcctatgttcctgactttctagttcaatttgataccaacgagtttgttctatatgaagtaaagtcagcttatgctaagaacaaacctgattttgatgttgaatgggaggcgaaagtaaaagcagcaactgaactagggctagaattggagcttgttgaagagagtgatattagggatacggttgtattaaataatcttaagcgcatgcatcgttatgcttcgaaagatgagctgaataacgtacataactctctcttaaaaataataaagtacaatggcgcccaatctgcaagatgcttgggagaacagttgggtttaaaaggccgaactgttttaccaattttgtgcgatttgctgtcaaggtgtttactcgatacacgtttggataagcctctatctcttgaatctcgatttgagttggcctcgtacggcggttcacccaagaaaaagcgcaaggtctaactagtaggaggctcgagatgggaccgaagaaaaagagaaaagtgggatccgcgatggctaagaaagggttctcaagtttccatagaaaagcagtctcttcccaagatacgcttgaatcgatagagcttgtctctagcgctaattgtctagaaagtgttacgtatcaagatatatcagcatttcccgaaacaattgcggtagagattaatttccgattaagcattcttcgctttttagcgcgaaagtgcgaaaccattgtggccaaatcaattgaaccacatcgtgtagagctacagcaaaactatagtagaaaaatacccagtgcaataacgatatatcgatggtggcttgcttttcgaaaatcagactacaaccccattagcttagcacctaatatcaaggatagaggtaatagagagacaaaagtgtcaacagttgttgattctattatggaacaggcagttgaaagagttatatctggacgaaaagtcaatgttagctctgcatataaacgtgttcgacgaaaagttcgtcaatacaatctaactcatggaacgaaatacacgtatcctaagtacgaatctgtaagaaagcgagtaaaaaagaaaaccccatttgagttattagccgcagggaaaggggagagagtagctaagagagagtttcgccgaatgggaaaaaagatcctcacgtctagcgtgctagagagggttgaaatagatcacactgtcgttgacctttttgcagtacacgaagagtatcgaatcccattgggccgaccttggcttactcaattggttgattgttacagtaaagctgtaatcggtttttatttaggtttcgagcctcctagctatgtgtcggtttcccttgcacttaagaatgcaatacaacgcaaagatgacttaatctcctcgtatgaatcgatcgagaatgaatggctatgttatggcatcccagacctactcgtaactgataatggtaaagagtttttgtcgaaagcatttgatcaagcatgtgaatcactattgatcaatgtgcatcaaaataaagttgagacgcccgacaacaaacctcatgttgaacgtaactacgggactattaatacttctctgttagacgatttacctgggaaatccttcagccagtaccttcaaagagaagggtacgactctgtgggagaagctacccttacactcaatgagattagagaaatttacttaatttggttggtggatatttatcataaaaaacccaatcagagaggcactaattgtcctaatgttgcgtggaaaaagggttgtcaagaatgggaaccagaggagttctctggttctaaagacgaattagactttaaatttgctattgttgattacaaacaacttactaaagtagggataactgtctacaaagaactgagttatagcaatgaccgtttagctgaatatagagggaagaaaggaaaccataaagttcagttcaagtataaccctgagtgtatggcagttatttgggtgttggatgaggatatgaatgagtactttacagttaatgcgattgactacgaatatgcaagtagagtatcactttggcaacataaatataacatgaaatatcaagcagaactaaattcagcagaatatgatgaggacaaggaaattgatgcagaaataaaaattgaagaaatcgcagatcgttcaattgttaagactaacaaaatcagagctcggaggcgtggcgctaggcatcaagagaatagcgcaagggctaagtcaatcagtaatgcgaacccggcctcgatacaaaaacatgaagatgaaatcgttagtgcagataatgacgattgggatattgattatgtctgagaaatcgtcaaatgggtcctaagaaaaagaggaaagtaggctcttccatgagtgaaacgcgtgaggctcgtatatcaagagctaaaagggcatttgtatccacaccgagcgttaggaaaatcttgagttacatggatagatgtagagatctatcagacctagagtctgagcctacatgcatgatggtctatggtgcttcgggtgtaggtaaaacgaccgtcatcaagaaatacttaaatcaaaacagaagagagtccgaagccgggggcgatataataccggttttgcatattgagttgccagacaatgcgaagccagtagatgcagcaagggaattgctggttgaaatgggtgacccgctagcactttatgaaactgacttagctagattgacgaaaagactgactgaattaatccctgcggtcggcgtgaagctgattattatcgatgagttccaacatttggtggaagaaaggtcaaatcgggttcttacccaagtaggtaattggctaaaaatgatacttaacaaaacgaaatgtccaattgttatatttggtatgccatactcaaaagttgtactgcaagcaaactcgcaacttcacgggcgattttccattcaggttgaactgcgcccctttagctaccagggaggtagaggtgtatttaaaacttttttggaataccttgataaagccctaccttttgaaaaacaggctggcttagccaacgaaagtttgcagaaaaaattgtatgcattctctcagggaaacatgcgttcgttgagaaaccttatttatcaagcatctatcgaagcaattgataatcagcatgagacgataaccgaagaagatttcgtttttgcatcgaagttgacatcgggcgataaacccaactcatggaaaaatccttttgaggagggtgttgaggtaacagaagatatgttacgaccgccaccaaaagatattggttgggaggactatttgagacattcaaccccgagagtgagtaaaccaggtagaaataaaaactttttcgaataacctaggctgctgcgctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcac

SEQ ID NO: 31 - DNA, 8234 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccctacttccaatccaatgccaccatgggcccgaaaaagaagcgaaaagttggctccggacgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttcgggaggtggttcgggtggctctggatcaagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgtcaggcggtggtagtggtgggagcggtagtgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtggcgcgcctagtggttcagaaacacccggcacatccgagtccgcgacacctgagagtggaggttctggaggttccggctcgagtggaatgaagctgccaaccaacctggcctacgagcggagcatcgaccccagcgacgtgtgcttcttcgtggtgtggccagacgataggaaaacccccctgacatacaattcccgcacactgctgggacagatggaggcagccagcctggcatacgacgtgtccggccagcctatcaagtctgccaccgcagaggccctggcacagggcaaccctcaccaggtggatttctgccatgtgccatacggcgccagccacatcgagtgtagcttctccgtgtcttttagctccgagctgcggcagccatataagtgtaattctagcaaggtgaagcagacactggtgcagctggtggagctgtacgaaaccaagatcggctggacagagctggccacccggtatctgatgaatatctgcaacggcaagtggctgtggaagaatacaagaaaggcctactgttggaacatcgtgctgaccccctggccttggaatggcgagaaagtgggcttcgaggacatcaggaccaactatacatcccgccaggacttcaagaacaataagaattggtctgccatcgtggagatgatcaagacagccttctcctctaccgacggcctggccatctttgaggtgagggccacactgcacctgccaaccaacgcaatggtgcgccctagccaggtgttcacagagaaggagagcggctccaagtctaagagcaagacccagaactctagggtgttccagagcaccacaatcgatggagagcggagcccaatcctgggagcctttaagacaggcgccgccatcgccaccatcgacgattggtatccagaggcaaccgagcctctgagggtgggccgctttggagtgcacagggaggacgtgacatgctacagacacccatctaccggcaaggatttctttagcatcctccagcaggccgagcactatatcgaggtgctgagtgccaacaagacacccgcccaggaaaccatcaatgacatgcacttcctgatggccaacctgatcaagggcggcatgtttcagcacaagggcgactaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 32 - DNA, 6773 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccctacttccaatccaatgccaccatgggcccgaaaaagaagcgaaaagttggctccggagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaggcgcgcctagtggttcagaaacacccggcacatccgagtccgcgacacctgagagtggaggttctgggagtggaatgaagctgccaaccaacctggcctacgagcggagcatcgaccccagcgacgtgtgcttcttcgtggtgtggccagacgataggaaaacccccctgacatacaattcccgcacactgctgggacagatggaggcagccagcctggcatacgacgtgtccggccagcctatcaagtctgccaccgcagaggccctggcacagggcaaccctcaccaggtggatttctgccatgtgccatacggcgccagccacatcgagtgtagcttctccgtgtcttttagctccgagctgcggcagccatataagtgtaattctagcaaggtgaagcagacactggtgcagctggtggagctgtacgaaaccaagatcggctggacagagctggccacccggtatctgatgaatatctgcaacggcaagtggctgtggaagaatacaagaaaggcctactgttggaacatcgtgctgaccccctggccttggaatggcgagaaagtgggcttcgaggacatcaggaccaactatacatcccgccaggacttcaagaacaataagaattggtctgccatcgtggagatgatcaagacagccttctcctctaccgacggcctggccatctttgaggtgagggccacactgcacctgccaaccaacgcaatggtgcgccctagccaggtgttcacagagaaggagagcggctccaagtctaagagcaagacccagaactctagggtgttccagagcaccacaatcgatggagagcggagcccaatcctgggagcctttaagacaggcgccgccatcgccaccatcgacgattggtatccagaggcaaccgagcctctgagggtgggccgctttggagtgcacagggaggacgtgacatgctacagacacccatctaccggcaaggatttctttagcatcctccagcaggccgagcactatatcgaggtgctgagtgccaacaagacacccgcccaggaaaccatcaatgacatgcacttcctgatggccaacctgatcaagggcggcatgtttcagcacaagggcgactaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 33 - DNA, 4801 bp, Artificial sequence
  Synthetic
ttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaatacgactcactatagggtctagaattcctgtgaactgccgagtaggtagctgataacaatgcaggcaaatcaaccttagtctgaaggccgtgaactgccgagtaggtagctgataacgttgcagcggccgctttttttcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctc

SEQ ID NO: 34 - DNA, 4609 bp, Artificial sequence; identical to SEQ ID NO: 15
  Synthetic

SEQ ID NO: 35 - DNA, 4801 bp, Artificial sequence
  Synthetic
ttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaatacgactcactatagggtctagaattcctgtgaactgccgagtaggtagctgataacaacatgttcctgatgaggtattatataggcctgtgaactgccgagtaggtagctgataacgttgcagcggccgctttttttcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctc

SEQ ID NO: 36 - DNA, 4801 bp, Artificial sequence
  Synthetic
ttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaatacgactcactatagggtctagaattcctgtgaactgccgagtaggtagctgataactatataatacctcatcaggaacatgttggtcggtgaactgccgagtaggtagctgataacgttgcagcggccgctttttttcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctc

SEQ ID NO: 37 - DNA, 4861 bp, Artificial sequence
  Synthetic
ttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaatacgactcactatagggtctagaattcctgtgaactgccgagtaggtagctgataactatataatacctcatcaggaacatgttggtcggtgaactgccgagtaggtagctgataacgctcggaggacagtactccgctcggaggacaggtgaactgccgagtaggtagctgataacgttgcagcggccgctttttttcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctc

SEQ ID NO: 38 - DNA, 6655 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggaatgttcctccagaggccaaagccctacagcgacgagtccctggagtctttctttatcagagtggccaataagaacggctatggcgatgtgcacaggttcctggaggccaccaagcggttcctccaggacatcgatcacaacggctaccagacctttccaacagacatcaccaggatcaatccctatagcgccaagaacagctcctctgcccgcacagcctccttcctgaagctggcccagctgacctttaatgagccacctgagctgctgggactggccatcaatcggacaaacatgaagtactccccttctaccagcgccgtggtgagaggagcagaggtgttcccacggtccctgctgagaacccacagcatcccatgctgtcccctgtgcctgagagagaacggctacgccagctatctgtggcacttccagggctacgagtattgtcactcccacaatgtgcctctgatcaccacatgctcttgtggcaaggagtttgactacagggtgagcggcctgaagggcatctgctgtaagtgcaaggagccaatcaccctgacaagccgcgagaatggccacgaggccgcctgtaccgtgtccaactggctggccggccacgagtctaagcctctgccaaacctgcccaagtcctatagatggggactggtgcactggtggatgggcatcaaggactccgagttcgatcacttctcttttgtgcagttctttagcaactggcctcggagcttccactccatcatcgaggacgaggtggagtttaatctggagcacgccgtggtgtccacctctgagctgcggctgaaggatctgctgggcagactgttctttggcagcatcaggctgccagagcgcaatctccagcacaacatcatcctgggcgagctgctgtgctacctggagaaccgcctgtggcaggacaagggcctgatcgccaatctgaagatgaacgccctggaggccacagtgatgctgaattgttccctggatcagatcgcctctatggtggagcagcgcatcctgaagcccaatagaaagtccaagcctaactctccactggacgtgaccgattacctgttccactttggcgacatcttctgcctgtggctggccgagtttcagtccgatgagttcaaccggagcttctacgtgagccggtggtaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 39 - DNA, 7393 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggaatgcagaccctgaaggagctgatcgccagcaaccccgacgatctgaccacagagctgaagagggccttccgccccctgacacctcacatcgccatcgacggcaatgagctggatgccctgaccatcctggtgaacctgacagacaagaccgacgatcagaaggacctgctggatcgggccaagtgtaagcagaagctgagagatgagaagtggtgggcctcctgcatcaactgcgtgaactacaggcagtctcacaacccaaagttccccgacatccgcagcgagggcgtgatcagaacacaggccctgggcgagctgcccagctttctgctgtctagctccaagatcccaccctaccactggtcttatagccacgactctaagtatgtgaataagagcgccttcctgaccaacgagttttgctgggatggcgagatcagctgtctgggcgagctgctgaaggacgccgatcaccctctgtggaacacactgaagaagctgggctgctcccagaaaacctgtaaggcaatggccaagcagctggccgacatcacactgaccacaatcaatgtgaccctggcccccaactacctgacacagatctctctgcctgactccgatacatcttatatctccctgtctcctgtggccagcctgtccatgcagagccacttccaccagaggctccaggatgagaacaggcactccgccatcacccggttcagccggaccacaaatatgggagtgacagcaatgacctgcggaggagccttcaggatgctgaaaagcggcgccaagttttctagccctccacaccaccggctgaacagcaagagatcctggctgacctccgagcatgtgcagtctctgaagcagtaccagcggctgaataagagcctgatcccagagaactccagaatcgccctgcggagaaagtataagatcgagctgcaaaatatggtgcgctcttggttcgccatgcaggaccacacactggatagcaatatcctgatccagcacctgaaccacgacctgtcctacctgggcgccaccaagcggttcgcctatgatcccgccatgacaaagctgtttaccgagctgctgaagagagagctgtctaacagcatcaacaatggcgagcagcacaccaatggcagctttctggtgctgcctaacatcagagtgtgcggagcaaccgccctgtcctctcctgtgacagtgggcatcccatccctgaccgccttctttggcttcgtgcacgcctttgagaggaatatcaaccgcaccacaagctccttcagggtggagagctttgccatctgcgtgcaccagctgcatgtggagaagcgcggcctgacagccgagttcgtggagaagggcgacggaacaatctccgccccagcaaccagggacgattggcagtgtgatgtggtgttctctctgatcctgaataccaactttgcccagcacatcgaccaggatacactcgtcacctctctgccaaagaggctggcaaggggaagcgccaagatcgccatcgacgatttcaagcacatcaactccttttctacactggaaaccgcaatcgagagcctgccaatcgaggcaggcagatggctgagtctgtacgcccagtccaacaataacctgtctgacctgctggccgccatgacagaggatcaccagctgatggccagctgcgtgggctaccacctgctggaggagcctaaagacaagccaaactccctcagaggctataagcacgccatcgccgagtgtatcatcggcctgatcaattccatcaccttctctagcgagacagatcccaacaccatcttttggagcctgaagaattaccagaactatctggtggtgcagcctcgctccatcaatgacgaaaccaccgataagtcctctctgtaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 40 - DNA, 6529 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggaatgaagctgccaaccaacctggcctacgagcggagcatcgaccccagcgacgtgtgcttcttcgtggtgtggccagacgataggaaaacccccctgacatacaattcccgcacactgctgggacagatggaggcagccagcctggcatacgacgtgtccggccagcctatcaagtctgccaccgcagaggccctggcacagggcaaccctcaccaggtggatttctgccatgtgccatacggcgccagccacatcgagtgtagcttctccgtgtcttttagctccgagctgcggcagccatataagtgtaattctagcaaggtgaagcagacactggtgcagctggtggagctgtacgaaaccaagatcggctggacagagctggccacccggtatctgatgaatatctgcaacggcaagtggctgtggaagaatacaagaaaggcctactgttggaacatcgtgctgaccccctggccttggaatggcgagaaagtgggcttcgaggacatcaggaccaactatacatcccgccaggacttcaagaacaataagaattggtctgccatcgtggagatgatcaagacagccttctcctctaccgacggcctggccatctttgaggtgagggccacactgcacctgccaaccaacgcaatggtgcgccctagccaggtgttcacagagaaggagagcggctccaagtctaagagcaagacccagaactctagggtgttccagagcaccacaatcgatggagagcggagcccaatcctgggagcctttaagacaggcgccgccatcgccaccatcgacgattggtatccagaggcaaccgagcctctgagggtgggccgctttggagtgcacagggaggacgtgacatgctacagacacccatctaccggcaaggatttctttagcatcctccagcaggccgagcactatatcgaggtgctgagtgccaacaagacacccgcccaggaaaccatcaatgacatgcacttcctgatggccaacctgatcaagggcggcatgtttcagcacaagggcgactaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 41 - DNA, 6070 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggagtgaagtggtactataagaccatcacattcctgcctgagctgtgcaacaatgagtctctggcagcaaagtgtctgcgggtgctgcacggcttcaattaccagtatgagacaagaaacatcggcgtgtcctttccactgtggtgcgacgccaccgtgggcaagaagatctctttcgtgagcaagaacaagatcgagctggatctgctgctgaagcagcactacttcgtgcagatggagcagctccagtattttcacatcagcaatacagtgctggtgcccgaggactgcacctacgtgagctttcggagatgtcagtccatcgataagctgaccgcagcaggactggcaagaaagatcaggcgcctggagaagcgggccctgtccagaggcgagcagttcgacccatctagctttgcccagaaggagcacacagccatcgcccactaccactctctgggcgagtcctctaagcagaccaatcggaacttcagactgaacatcaggatgctgagtgagcagccaagggagggcaattccatcttcagctcctatggcctgagcaattccgagaactcttttcagcccgtgcctctgatctaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 42 - DNA, 7282 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggaatggccaagaagggcttcagctcctttcaccgcaaggccgtgtctagccaggacaccctggagagcatcgagctggtgtcctctgccaactgcctggagagcgtgacataccaggacatctccgccttccctgaaaccatcgccgtggagatcaatttcagactgagcatcctgagatttctggccaggaagtgtgagacaatcgtggccaagtccatcgagcctcaccgggtggagctgcaacagaactatagccggaagatcccaagcgccatcaccatctaccgctggtggctggcctttcggaaaagcgactataatccaatctccctggcccccaacatcaaggatagaggcaatagggagacaaaggtgtccaccgtggtggactctatcatggagcaggcagtggagcgcgtgatcagcggacggaaggtgaacgtgagttccgcctataagagagtgcggagaaaggtgaggcagtacaatctgacccacggcacaaagtacacctatcccaagtacgagtccgtgagaaagagggtgaagaagaaaacccctttcgagctgctggcagcaggcaagggagagagggtggccaagcgggagtttaggcgcatgggcaagaagatcctgacatctagcgtgctggagcgggtggagatcgaccacaccgtggtggatctgttcgccgtgcacgaggagtatagaatccctctgggcaggccctggctgacccagctggtggactgctactctaaggccgtgatcggcttctatctgggctttgagccccctagctacgtgtctgtgagcctggccctgaagaacgccatccagagaaaggacgatctgatctcctcttatgagagcatcgagaacgagtggctgtgctacggcatcccagacctgctggtgacagataatggcaaggagttcctgagcaaggcctttgaccaggcctgtgagtccctgctgatcaacgtgcaccagaataaggtggagacacccgataataagcctcacgtcgagaggaactacggcacaatcaataccagcctgctggacgatctgcccggcaagtccttctctcagtacctccagagggagggatatgactccgtgggagaggccaccctgacactgaacgagatcagagagatctatctgatctggctggtggatatttaccacaagaagcccaatcagcgcggcaccaactgccctaatgtggcctggaagaagggctgtcaggagtgggagcctgaagagtttagcggctccaaggacgagctggatttcaagtttgccatcgtggactacaagcagctgacaaaagtgggcatcaccgtgtacaaggagctgtcttatagcaacgataggctggcagagtacaggggcaagaagggaaaccacaaggtgcagttcaagtataatccagagtgtatggccgtgatctgggtgctggacgaggatatgaacgagtactttacagtgaatgccatcgattacgagtatgcctctcgggtgagcctgtggcagcacaagtacaacatgaagtatcaggccgagctgaatagcgccgagtatgacgaggataaggagatcgacgccgagatcaagatcgaggagatcgccgatagatccatcgtgaaaaccaacaagatcagggcacggagaaggggagcaagacaccaggagaactctgccagggccaagtccatctctaacgccaatcctgcctctatccagaagcacgaggacgagatcgtgagcgccgataacgacgattgggacatcgattacgtgtaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 43 - DNA, 6157 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccctacttccaatccaatgccaccatgggcgccacatccctgccaaccccctctgccatcaccacaagcgccctggagtacgccttccacacaccagcaaggaacctgaccaagtcccgcggcaagaatatccacagatacgtgagcgtgaagatgagcaagaggatcacagtggagtccaccctggagtgcgacgcctgttaccacttcgattttgagccatctatcgtgcgcttctgtgcccagcccatccggtttctgtactatctgaacggccagagccactcctatgtgcctgacttcctggtgcagtttgataccaacgagttcgtgctgtacgaggtgaaaagcgcctatgccaagaataagccagactttgatgtggagtgggaggccaaggtgaaggcagcaacagagctgggcctggagctggagctggtggaggagtccgacatccgcgataccgtggtgctgaacaatctgaagcgcatgcaccgctacgcctccaaggacgagctgaacaatgtgcacaactctctgctgaagatcatcaagtataatggagcacagtccgccaggtgcctgggagagcagctgggcctgaagggaaggacagtgctgccaatcctgtgcgatctgctgtctaggtgtctgctggacacccgcctggataagcccctgtccctggagtctcggttcgagctggcaagctacggaggctccggaaaacggacagccgacggaagcgagttcgagtcaccgaaaaagaagcgaaaagtttaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 44 - DNA, 6457 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccctacttccaatccaatgccaccatgggctccgagacaagagaggcaaggatctctcgcgcaaagagggcctttgtgagcacaccctccgtgcgcaagatcctgtcctatatggatcgctgccgggacctgtccgatctggagtctgagcctacctgtatgatggtgtacggcgcctctggcgtgggcaagaccacagtgatcaagaagtatctgaatcagaataggagggagagcgaggcaggaggcgacatcatccccgtgctgcacatcgagctgcctgacaacgccaagccagtggatgccgccagagagctgctggtggagatgggcgaccctctggccctgtacgagacagatctggccagactgacaaagaggctgaccgagctgatcccagcagtgggagtgaagctgatcatcatcgatgagttccagcacctggtggaggagagaagcaacagggtgctgacacaagtgggcaactggctgaagatgatcctgaataagaccaagtgccctatcgtgatctttggcatgccatatagcaaggtggtgctccaggccaattctcagctgcacggcagattcagcatccaggtggagctgaggccattttcctaccagggaggaaggggcgtgttcaagacctttctggagtatctggacaaggccctgcccttcgagaagcaggcaggactggccaacgagtctctccagaagaagctgtacgccttttcccagggcaacatgcgctctctgcggaatctgatctatcaggcctccatcgaggccatcgacaatcagcacgaaaccatcacagaggaggatttcgtgtttgccagcaagctgacatccggcgacaagccaaacagctggaagaatcccttcgaggagggcgtggaggtgacagaggatatgctgcggccaccccccaaggacatcggctgggaggattacctgagacactccacccccagggtgtctaagcctggcagaaacaagaatttctttgagggctccggaaaacggacagccgacggaagcgagttcgagtcaccgaaaaagaagcgaaaagtttaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 45 - DNA, 7985 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccagccaccatggccacatccctgccaaccccctctgccatcaccacaagcgccctggagtacgccttccacacaccagcaaggaacctgaccaagtcccgcggcaagaatatccacagatacgtgagcgtgaagatgagcaagaggatcacagtggagtccaccctggagtgcgacgcctgttaccacttcgattttgagccatctatcgtgcgcttctgtgcccagcccatccggtttctgtactatctgaacggccagagccactcctatgtgcctgacttcctggtgcagtttgataccaacgagttcgtgctgtacgaggtgaaaagcgcctatgccaagaataagccagactttgatgtggagtgggaggccaaggtgaaggcagcaacagagctgggcctggagctggagctggtggaggagtccgacatccgcgataccgtggtgctgaacaatctgaagcgcatgcaccgctacgcctccaaggacgagctgaacaatgtgcacaactctctgctgaagatcatcaagtataatggagcacagtccgccaggtgcctgggagagcagctgggcctgaagggaaggacagtgctgccaatcctgtgcgatctgctgtctaggtgtctgctggacacccgcctggataagcccctgtccctggagtctcggttcgagctggcaagctacggaggcagcggctccggaaaacggacagccgacggaagcgagttcgagtcaccaaagaagaagcggaaagtcggatccggctctggaggcgccaagaagggcttcagctcctttcaccgcaaggccgtgtctagccaggacaccctggagagcatcgagctggtgtcctctgccaactgcctggagagcgtgacataccaggacatctccgccttccctgaaaccatcgccgtggagatcaatttcagactgagcatcctgagatttctggccaggaagtgtgagacaatcgtggccaagtccatcgagcctcaccgggtggagctgcaacagaactatagccggaagatcccaagcgccatcaccatctaccgctggtggctggcctttcggaaaagcgactataatccaatctccctggcccccaacatcaaggatagaggcaatagggagacaaaggtgtccaccgtggtggactctatcatggagcaggcagtggagcgcgtgatcagcggacggaaggtgaacgtgagttccgcctataagagagtgcggagaaaggtgaggcagtacaatctgacccacggcacaaagtacacctatcccaagtacgagtccgtgagaaagagggtgaagaagaaaacccctttcgagctgctggcagcaggcaagggagagagggtggccaagcgggagtttaggcgcatgggcaagaagatcctgacatctagcgtgctggagcgggtggagatcgaccacaccgtggtggatctgttcgccgtgcacgaggagtatagaatccctctgggcaggccctggctgacccagctggtggactgctactctaaggccgtgatcggcttctatctgggctttgagccccctagctacgtgtctgtgagcctggccctgaagaacgccatccagagaaaggacgatctgatctcctcttatgagagcatcgagaacgagtggctgtgctacggcatcccagacctgctggtgacagataatggcaaggagttcctgagcaaggcctttgaccaggcctgtgagtccctgctgatcaacgtgcaccagaataaggtggagacacccgataataagcctcacgtcgagaggaactacggcacaatcaataccagcctgctggacgatctgcccggcaagtccttctctcagtacctccagagggagggatatgactccgtgggagaggccaccctgacactgaacgagatcagagagatctatctgatctggctggtggatatttaccacaagaagcccaatcagcgcggcaccaactgccctaatgtggcctggaagaagggctgtcaggagtgggagcctgaagagtttagcggctccaaggacgagctggatttcaagtttgccatcgtggactacaagcagctgacaaaagtgggcatcaccgtgtacaaggagctgtcttatagcaacgataggctggcagagtacaggggcaagaagggaaaccacaaggtgcagttcaagtataatccagagtgtatggccgtgatctgggtgctggacgaggatatgaacgagtactttacagtgaatgccatcgattacgagtatgcctctcgggtgagcctgtggcagcacaagtacaacatgaagtatcaggccgagctgaatagcgccgagtatgacgaggataaggagatcgacgccgagatcaagatcgaggagatcgccgatagatccatcgtgaaaaccaacaagatcagggcacggagaaggggagcaagacaccaggagaactctgccagggccaagtccatctctaacgccaatcctgcctctatccagaagcacgaggacgagatcgtgagcgccgataacgacgattgggacatcgattacgtgtaactgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 46 - DNA, 6727 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggagactacaaggaccacgacggtgactacaaggaccacgacatcgactacaaggacgacgacgacaagtccggaatgttcctccagaggccaaagccctacagcgacgagtccctggagtctttctttatcagagtggccaataagaacggctatggcgatgtgcacaggttcctggaggccaccaagcggttcctccaggacatcgatcacaacggctaccagacctttccaacagacatcaccaggatcaatccctatagcgccaagaacagctcctctgcccgcacagcctccttcctgaagctggcccagctgacctttaatgagccacctgagctgctgggactggccatcaatcggacaaacatgaagtactccccttctaccagcgccgtggtgagaggagcagaggtgttcccacggtccctgctgagaacccacagcatcccatgctgtcccctgtgcctgagagagaacggctacgccagctatctgtggcacttccagggctacgagtattgtcactcccacaatgtgcctctgatcaccacatgctcttgtggcaaggagtttgactacagggtgagcggcctgaagggcatctgctgtaagtgcaaggagccaatcaccctgacaagccgcgagaatggccacgaggccgcctgtaccgtgtccaactggctggccggccacgagtctaagcctctgccaaacctgcccaagtcctatagatggggactggtgcactggtggatgggcatcaaggactccgagttcgatcacttctcttttgtgcagttctttagcaactggcctcggagcttccactccatcatcgaggacgaggtggagtttaatctggagcacgccgtggtgtccacctctgagctgcggctgaaggatctgctgggcagactgttctttggcagcatcaggctgccagagcgcaatctccagcacaacatcatcctgggcgagctgctgtgctacctggagaaccgcctgtggcaggacaagggcctgatcgccaatctgaagatgaacgccctggaggccacagtgatgctgaattgttccctggatcagatcgcctctatggtggagcagcgcatcctgaagcccaatagaaagtccaagcctaactctccactggacgtgaccgattacctgttccactttggcgacatcttctgcctgtggctggccgagtttcagtccgatgagttcaaccggagcttctacgtgagccggtggtaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 47 - DNA, 7465 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggagactacaaggaccacgacggtgactacaaggaccacgacatcgactacaaggacgacgacgacaagtccggaatgcagaccctgaaggagctgatcgccagcaaccccgacgatctgaccacagagctgaagagggccttccgccccctgacacctcacatcgccatcgacggcaatgagctggatgccctgaccatcctggtgaacctgacagacaagaccgacgatcagaaggacctgctggatcgggccaagtgtaagcagaagctgagagatgagaagtggtgggcctcctgcatcaactgcgtgaactacaggcagtctcacaacccaaagttccccgacatccgcagcgagggcgtgatcagaacacaggccctgggcgagctgcccagctttctgctgtctagctccaagatcccaccctaccactggtcttatagccacgactctaagtatgtgaataagagcgccttcctgaccaacgagttttgctgggatggcgagatcagctgtctgggcgagctgctgaaggacgccgatcaccctctgtggaacacactgaagaagctgggctgctcccagaaaacctgtaaggcaatggccaagcagctggccgacatcacactgaccacaatcaatgtgaccctggcccccaactacctgacacagatctctctgcctgactccgatacatcttatatctccctgtctcctgtggccagcctgtccatgcagagccacttccaccagaggctccaggatgagaacaggcactccgccatcacccggttcagccggaccacaaatatgggagtgacagcaatgacctgcggaggagccttcaggatgctgaaaagcggcgccaagttttctagccctccacaccaccggctgaacagcaagagatcctggctgacctccgagcatgtgcagtctctgaagcagtaccagcggctgaataagagcctgatcccagagaactccagaatcgccctgcggagaaagtataagatcgagctgcaaaatatggtgcgctcttggttcgccatgcaggaccacacactggatagcaatatcctgatccagcacctgaaccacgacctgtcctacctgggcgccaccaagcggttcgcctatgatcccgccatgacaaagctgtttaccgagctgctgaagagagagctgtctaacagcatcaacaatggcgagcagcacaccaatggcagctttctggtgctgcctaacatcagagtgtgcggagcaaccgccctgtcctctcctgtgacagtgggcatcccatccctgaccgccttctttggcttcgtgcacgcctttgagaggaatatcaaccgcaccacaagctccttcagggtggagagctttgccatctgcgtgcaccagctgcatgtggagaagcgcggcctgacagccgagttcgtggagaagggcgacggaacaatctccgccccagcaaccagggacgattggcagtgtgatgtggtgttctctctgatcctgaataccaactttgcccagcacatcgaccaggatacactcgtcacctctctgccaaagaggctggcaaggggaagcgccaagatcgccatcgacgatttcaagcacatcaactccttttctacactggaaaccgcaatcgagagcctgccaatcgaggcaggcagatggctgagtctgtacgcccagtccaacaataacctgtctgacctgctggccgccatgacagaggatcaccagctgatggccagctgcgtgggctaccacctgctggaggagcctaaagacaagccaaactccctcagaggctataagcacgccatcgccgagtgtatcatcggcctgatcaattccatcaccttctctagcgagacagatcccaacaccatcttttggagcctgaagaattaccagaactatctggtggtgcagcctcgctccatcaatgacgaaaccaccgataagtcctctctgtaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 48 - DNA, 6601 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggagactacaaggaccacgacggtgactacaaggaccacgacatcgactacaaggacgacgacgacaagtccggaatgaagctgccaaccaacctggcctacgagcggagcatcgaccccagcgacgtgtgcttcttcgtggtgtggccagacgataggaaaacccccctgacatacaattcccgcacactgctgggacagatggaggcagccagcctggcatacgacgtgtccggccagcctatcaagtctgccaccgcagaggccctggcacagggcaaccctcaccaggtggatttctgccatgtgccatacggcgccagccacatcgagtgtagcttctccgtgtcttttagctccgagctgcggcagccatataagtgtaattctagcaaggtgaagcagacactggtgcagctggtggagctgtacgaaaccaagatcggctggacagagctggccacccggtatctgatgaatatctgcaacggcaagtggctgtggaagaatacaagaaaggcctactgttggaacatcgtgctgaccccctggccttggaatggcgagaaagtgggcttcgaggacatcaggaccaactatacatcccgccaggacttcaagaacaataagaattggtctgccatcgtggagatgatcaagacagccttctcctctaccgacggcctggccatctttgaggtgagggccacactgcacctgccaaccaacgcaatggtgcgccctagccaggtgttcacagagaaggagagcggctccaagtctaagagcaagacccagaactctagggtgttccagagcaccacaatcgatggagagcggagcccaatcctgggagcctttaagacaggcgccgccatcgccaccatcgacgattggtatccagaggcaaccgagcctctgagggtgggccgctttggagtgcacagggaggacgtgacatgctacagacacccatctaccggcaaggatttctttagcatcctccagcaggccgagcactatatcgaggtgctgagtgccaacaagacacccgcccaggaaaccatcaatgacatgcacttcctgatggccaacctgatcaagggcggcatgtttcagcacaagggcgactaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 49 - DNA, 6142 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggagactacaaggaccacgacggtgactacaaggaccacgacatcgactacaaggacgacgacgacaagtccggagtgaagtggtactataagaccatcacattcctgcctgagctgtgcaacaatgagtctctggcagcaaagtgtctgcgggtgctgcacggcttcaattaccagtatgagacaagaaacatcggcgtgtcctttccactgtggtgcgacgccaccgtgggcaagaagatctctttcgtgagcaagaacaagatcgagctggatctgctgctgaagcagcactacttcgtgcagatggagcagctccagtattttcacatcagcaatacagtgctggtgcccgaggactgcacctacgtgagctttcggagatgtcagtccatcgataagctgaccgcagcaggactggcaagaaagatcaggcgcctggagaagcgggccctgtccagaggcgagcagttcgacccatctagctttgcccagaaggagcacacagccatcgcccactaccactctctgggcgagtcctctaagcagaccaatcggaacttcagactgaacatcaggatgctgagtgagcagccaagggagggcaattccatcttcagctcctatggcctgagcaattccgagaactcttttcagcccgtgcctctgatctaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 50 - DNA, 6740 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggccaccatgggaagcggcgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaggtggtagtggtgggagcggtggcaaacggacagccgacggaagcgagttcgagtcaccgaaaaagaagcgaaaagttggctccggaatgaagctgccaaccaacctggcctacgagcggagcatcgaccccagcgacgtgtgcttcttcgtggtgtggccagacgataggaaaacccccctgacatacaattcccgcacactgctgggacagatggaggcagccagcctggcatacgacgtgtccggccagcctatcaagtctgccaccgcagaggccctggcacagggcaaccctcaccaggtggatttctgccatgtgccatacggcgccagccacatcgagtgtagcttctccgtgtcttttagctccgagctgcggcagccatataagtgtaattctagcaaggtgaagcagacactggtgcagctggtggagctgtacgaaaccaagatcggctggacagagctggccacccggtatctgatgaatatctgcaacggcaagtggctgtggaagaatacaagaaaggcctactgttggaacatcgtgctgaccccctggccttggaatggcgagaaagtgggcttcgaggacatcaggaccaactatacatcccgccaggacttcaagaacaataagaattggtctgccatcgtggagatgatcaagacagccttctcctctaccgacggcctggccatctttgaggtgagggccacactgcacctgccaaccaacgcaatggtgcgccctagccaggtgttcacagagaaggagagcggctccaagtctaagagcaagacccagaactctagggtgttccagagcaccacaatcgatggagagcggagcccaatcctgggagcctttaagacaggcgccgccatcgccaccatcgacgattggtatccagaggcaaccgagcctctgagggtgggccgctttggagtgcacagggaggacgtgacatgctacagacacccatctaccggcaaggatttctttagcatcctccagcaggccgagcactatatcgaggtgctgagtgccaacaagacacccgcccaggaaaccatcaatgacatgcacttcctgatggccaacctgatcaagggcggcatgtttcagcacaagggcgactaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 51 - DNA, 6659 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccctacttccaatccaatgccaccatgggctccgagacaagagaggcaaggatctctcgcgcaaagagggcctttgtgagcacaccctccgtgcgcaagatcctgtcctatatggatcgctgccgggacctgtccgatctggagtctgagcctacctgtatgatggtgtacggcgcctctggcgtgggcaagaccacagtgatcaagaagtatctgaatcagaataggagggagagcgaggcaggaggcgacatcatccccgtgctgcacatcgagctgcctgacaacgccaagccagtggatgccgccagagagctgctggtggagatgggcgaccctctggccctgtacgagacagatctggccagactgacaaagaggctgaccgagctgatcccagcagtgggagtgaagctgatcatcatcgatgagttccagcacctggtggaggagagaagcaacagggtgctgacacaagtgggcaactggctgaagatgatcctgaataagaccaagtgccctatcgtgatctttggcatgccatatagcaaggtggtgctccaggccaattctcagctgcacggcagattcagcatccaggtggagctgaggccattttcctaccagggaggaaggggcgtgttcaagacctttctggagtatctggacaaggccctgcccttcgagaagcaggcaggactggccaacgagtctctccagaagaagctgtacgccttttcccagggcaacatgcgctctctgcggaatctgatctatcaggcctccatcgaggccatcgacaatcagcacgaaaccatcacagaggaggatttcgtgtttgccagcaagctgacatccggcgacaagccaaacagctggaagaatcccttcgaggagggcgtggaggtgacagaggatatgctgcggccaccccccaaggacatcggctgggaggattacctgagacactccacccccagggtgtctaagcctggcagaaacaagaatttctttgagggctccggaaaacggacagccgacggaagcgagttcgagtcaccgaaaaagaagcgaaaagttggtggtagtggtggaagcggcgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagataaagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 52 - DNA, 6644 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggagactacaaggaccacgacggtgactacaaggaccacgacatcgactacaaggacgacgacgacaagtccggaatgaagctgccaaccaacctggcctacgagcggagcatcgaccccagcgacgtgtgcttcttcgtggtgtggccagacgataggaaaacccccctgacatacaattcccgcacactgctgggacagatggaggcagccagcctggcatacgacgtgtccggccagcctatcaagtctgccaccgcagaggccctggcacagggcaaccctcaccaggtggatttctgccatgtgccatacggcgccagccacatcgagtgtagcttctccgtgtcttttagctccgagctgcggcagccatataagtgtaattctagcaaggtgaagcagacactggtgcagctggtggagctgtacgaaaccaagatcggctggacagagctggccacccggtatctgatgaatatctgcaacggcaagtggctgtggaagaatacaagaaaggcctactgttggaacatcgtgctgaccccctggccttggaatggcgagaaagtgggcttcgaggacatcaggaccaactatacatcccgccaggacttcaagaacaataagaattggtctgccatcgtggagatgatcaagacagccttctcctctaccgacggcctggccatctttgaggtgagggccacactgcacctgccaaccaacgcaatggtgcgccctagccaggtgttcacagagaaggagagcggctccaagtctaagagcaagacccagaactctagggtgttccagagcaccacaatcgatggagagcggagcccaatcctgggagcctttaagacaggcgccgccatcgccaccatcgacgattggtatccagaggcaaccgagcctctgagggtgggccgctttggagtgcacagggaggacgtgacatgctacagacacccatctaccggcaaggatttctttagcatcctccagcaggccgagcactatatcgaggtgctgagtgccaacaagacacccgcccaggaaaccatcaatgacatgcacttcctgatggccaacctgatcaagggcggcatgtttcagcacaagggcgacggctccggcaaaagaaccgccgacggcagcgaattcgagtcacccaagaagaagaggaaagtctgactagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 53 - DNA, 6592 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggcaaaagaaccgccgacggcagcgaattcgagtcacccaagaagaagaggaaagtcggctccggaatgaagctgccaaccaacctggcctacgagcggagcatcgaccccagcgacgtgtgcttcttcgtggtgtggccagacgataggaaaacccccctgacatacaattcccgcacactgctgggacagatggaggcagccagcctggcatacgacgtgtccggccagcctatcaagtctgccaccgcagaggccctggcacagggcaaccctcaccaggtggatttctgccatgtgccatacggcgccagccacatcgagtgtagcttctccgtgtcttttagctccgagctgcggcagccatataagtgtaattctagcaaggtgaagcagacactggtgcagctggtggagctgtacgaaaccaagatcggctggacagagctggccacccggtatctgatgaatatctgcaacggcaagtggctgtggaagaatacaagaaaggcctactgttggaacatcgtgctgaccccctggccttggaatggcgagaaagtgggcttcgaggacatcaggaccaactatacatcccgccaggacttcaagaacaataagaattggtctgccatcgtggagatgatcaagacagccttctcctctaccgacggcctggccatctttgaggtgagggccacactgcacctgccaaccaacgcaatggtgcgccctagccaggtgttcacagagaaggagagcggctccaagtctaagagcaagacccagaactctagggtgttccagagcaccacaatcgatggagagcggagcccaatcctgggagcctttaagacaggcgccgccatcgccaccatcgacgattggtatccagaggcaaccgagcctctgagggtgggccgctttggagtgcacagggaggacgtgacatgctacagacacccatctaccggcaaggatttctttagcatcctccagcaggccgagcactatatcgaggtgctgagtgccaacaagacacccgcccaggaaaccatcaatgacatgcacttcctgatggccaacctgatcaagggcggcatgtttcagcacaagggcgactaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 54 - DNA, 6655 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggcaagcggactgctgatggcagtgaatttgagtccccaaagaagaagagaaaggtggggagcggcaaaagaaccgccgacggcagcgaattcgagtcacccaagaagaagaggaaagtcggctccggaatgaagctgccaaccaacctggcctacgagcggagcatcgaccccagcgacgtgtgcttcttcgtggtgtggccagacgataggaaaacccccctgacatacaattcccgcacactgctgggacagatggaggcagccagcctggcatacgacgtgtccggccagcctatcaagtctgccaccgcagaggccctggcacagggcaaccctcaccaggtggatttctgccatgtgccatacggcgccagccacatcgagtgtagcttctccgtgtcttttagctccgagctgcggcagccatataagtgtaattctagcaaggtgaagcagacactggtgcagctggtggagctgtacgaaaccaagatcggctggacagagctggccacccggtatctgatgaatatctgcaacggcaagtggctgtggaagaatacaagaaaggcctactgttggaacatcgtgctgaccccctggccttggaatggcgagaaagtgggcttcgaggacatcaggaccaactatacatcccgccaggacttcaagaacaataagaattggtctgccatcgtggagatgatcaagacagccttctcctctaccgacggcctggccatctttgaggtgagggccacactgcacctgccaaccaacgcaatggtgcgccctagccaggtgttcacagagaaggagagcggctccaagtctaagagcaagacccagaactctagggtgttccagagcaccacaatcgatggagagcggagcccaatcctgggagcctttaagacaggcgccgccatcgccaccatcgacgattggtatccagaggcaaccgagcctctgagggtgggccgctttggagtgcacagggaggacgtgacatgctacagacacccatctaccggcaaggatttctttagcatcctccagcaggccgagcactatatcgaggtgctgagtgccaacaagacacccgcccaggaaaccatcaatgacatgcacttcctgatggccaacctgatcaagggcggcatgtttcagcacaagggcgactaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 55 - DNA, 6718 bp, Artificial sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgggcaaacggacagccgacggaagcgagttcgagtcaccgaaaaagaagcgaaaagttggctccggcaagcgcaccgccgatggttccgagttcgaaagccccaaaaaaaagcgcaaggtcgggtccggcaagcggactgctgatggcagtgaatttgagtccccaaagaagaagagaaaggtggggagcggcaaaagaaccgccgacggcagcgaattcgagtcacccaagaagaagaggaaagtcggctccggaatgaagctgccaaccaacctggcctacgagcggagcatcgaccccagcgacgtgtgcttcttcgtggtgtggccagacgataggaaaacccccctgacatacaattcccgcacactgctgggacagatggaggcagccagcctggcatacgacgtgtccggccagcctatcaagtctgccaccgcagaggccctggcacagggcaaccctcaccaggtggatttctgccatgtgccatacggcgccagccacatcgagtgtagcttctccgtgtcttttagctccgagctgcggcagccatataagtgtaattctagcaaggtgaagcagacactggtgcagctggtggagctgtacgaaaccaagatcggctggacagagctggccacccggtatctgatgaatatctgcaacggcaagtggctgtggaagaatacaagaaaggcctactgttggaacatcgtgctgaccccctggccttggaatggcgagaaagtgggcttcgaggacatcaggaccaactatacatcccgccaggacttcaagaacaataagaattggtctgccatcgtggagatgatcaagacagccttctcctctaccgacggcctggccatctttgaggtgagggccacactgcacctgccaaccaacgcaatggtgcgccctagccaggtgttcacagagaaggagagcggctccaagtctaagagcaagacccagaactctagggtgttccagagcaccacaatcgatggagagcggagcccaatcctgggagcctttaagacaggcgccgccatcgccaccatcgacgattggtatccagaggcaaccgagcctctgagggtgggccgctttggagtgcacagggaggacgtgacatgctacagacacccatctaccggcaaggatttctttagcatcctccagcaggccgagcactatatcgaggtgctgagtgccaacaagacacccgcccaggaaaccatcaatgacatgcacttcctgatggccaacctgatcaagggcggcatgtttcagcacaagggcgactaattacattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2024/0209399 A1
(43) Pub. Date: Jun. 27, 2024

(54) SYSTEMS, METHODS, AND COMPONENTS FOR RNA-GUIDED EFFECTOR RECRUITMENT

(71) Applicant: The Trustees of Columbia University in the City of New York, New York, NY (US)

(72) Inventors: Samuel Henry Sternberg, New York, NY (US); George Davis Lampe, New York, NY (US); Rebeca Teresa King Davidson, New York, NY (US)

(21) Appl. No.: 18/570,914

(22) PCT Filed: Jun. 17, 2022
(86) PCT No.: PCT/US22/34072

§ 371 Date: Dec. 15, 2023

Related U.S. Application Data
(60) Provisional application No. 63/211,635, filed on Jun. 17, 2021.

(30) Foreign Application Priority Data
Jun. 17, 2021 (US) 63211635

(51) Int. Cl. C12N 15/90; C12N 9/22; C12N 15/11

(52) U.S. Cl. CPC C12N 15/907; C12N 9/22; C12N 15/11; C12N 2310/20; C12N 2800/80; C12N 2800/90

(57) ABSTRACT

The present disclosure provides systems, kits, and methods provide systems and methods for recruiting one or more effector domains to a target nucleic acid and or modulating expression of a target gene in a cell utilizing an engineered Clustered Regularly Interspaced Short Palindromic Repeats (CRISPR)-CRISPR associated (Cas) system. More particularly, the present disclosure provides systems comprising: an engineered CRISPR-Cas system or one or more nucleic acids encoding the engineered CRISPR-Cas system, wherein the CRISPR-Cas system comprises: at least one Cas protein (e.g., Cas6, Cas7, Cas5, Cas8 and/or Cas12k); a guide RNA (gRNA) complementary to at least a portion of the target nucleic acid sequence; and, optionally, at least one transposon-associated protein (e.g., TniQ, TnsC, TnsA, and/or TnsB).

### CROSS-REFERENCE TO RELATED APPLICATIONS

This application claims the benefit of U.S. Provisional Application No. 63/211,635, filed Jun. 17, 2021, the content of which is herein incorporated by reference in their entirety.

### STATEMENT REGARDING FEDERALLY-SPONSORED RESEARCH OR DEVELOPMENT

This invention was made with government support under grant number HG011650 awarded by the National Institutes of Health. The government has certain rights in the invention.

### FIELD

The present invention relates to methods and systems for RNA-guided recruitment of effector molecules (e.g., transcriptional activators, repressors, epigenetic modifiers) to a target nucleic acid.

### SEQUENCE LISTING STATEMENT

The text of the computer readable sequence listing filed herewith, titled “39604-601_SEQUENCE_LISTING_ST25”, created Jun. 17, 2022, having a file size of 430,408 bytes, is hereby incorporated by reference in its entirety.

### BACKGROUND

The ability to precisely and efficiently edit DNA sequences and control gene expression within living cells has been an ultimate goal of life science research for decades and can provide dramatic insight into genetic influences of many diseases. RNA-programmable CRISPR-associated (Cas) nucleases have contributed to the pursuit of this goal through their ability to generate a double stranded DNA break (DSB) at a precise target location in the genome of a wide variety of cells and organisms. In addition, catalytically inactivated Cas nucleases are also useful as programmable DNA-binding proteins that localize tethered proteins to target DNA loci.

### SUMMARY

Provided herein are systems for effector domain recruitment to a target nucleic acid to a target nucleic acid.

In some embodiments, the systems comprise an engineered Clustered Regularly Interspaced Short Palindromic Repeats (CRISPR)-CRISPR associated (Cas) system or one or more nucleic acids encoding the engineered CRISPR-Cas system, wherein the CRISPR-Cas system comprises: a) at least one Cas protein and b) a guide RNA (gRNA) complementary to at least a portion of the target nucleic acid sequence, wherein one or more of the at least one Cas protein comprises at least one effector domain. The system may further comprise at least one transposon-associated protein, or one or more nucleic acids encoding thereof, wherein one or more of the at least one transposon-associated protein comprises at least one effector domain.

In some embodiments, the systems comprise an engineered Clustered Regularly Interspaced Short Palindromic Repeats (CRISPR)-CRISPR associated (Cas) system or one or more nucleic acids encoding the engineered CRISPR-Cas system, wherein the CRISPR-Cas system comprises: a) at least one Cas protein; b) at least one transposon-associated protein; and c) a guide RNA (gRNA) complementary to at least a portion of the target nucleic acid sequence, wherein one or more of the at least one Cas protein and the at least one transposon-associated protein comprises at least one effector domain.

In some embodiments, the target nucleic acid comprises a promoter region of a gene of interest. In some embodiments, the target nucleic acid comprises an upstream activator sequence. In some embodiments, the gene of interest is located on a chromosome in a cell.

In some embodiments one or more of the at least one Cas protein are part of a ribonucleoprotein complex with the gRNA.

In some embodiments, the at least one effector domain comprises a transcription activator, a transcription repressor, a base editor, an epigenetic modifier, a chromosomal locus imaging agent, or a combination thereof.

In some embodiments, the at least one Cas protein is derived from a Type I or Type V CRISPR-Cas system. In some embodiments, the at least one Cas protein comprises Cas5, Cas6, Cas7, and Cas8. In some embodiments, the at least one Cas protein comprises a Cas8-Cas5 fusion protein. In some embodiments, the at least one Cas protein comprises Cas12k.

In some embodiments, effector domain(s) may be appended to Cas7, Cas8, Cas8-Cas5, or any combination thereof. In some embodiments, effector domain(s) may be appended to Cas12k.

In some embodiments, the at least one transposon protein is derived from a Tn7 or Tn7-like transposon system. In some embodiments, the at least one transposon-associated protein comprises TniQ. In some embodiments, the at least one transposon-associated protein further comprises TnsC. In some embodiments, the at least one transposon associated protein further comprises TnsA, TnsB, or a combination thereof.

In some embodiments, effector domain(s) may be appended to TniQ, TnsC, or a combination thereof.

In some embodiments, the at least one transposon protein comprises a TnsA-TnsB fusion protein. In some embodiments, the TnsA-TnsB fusion protein further comprises an amino acid linker between TnsA and TnsB. The linker may be a flexible linker. In some embodiments, the linker comprises at least one glycine-rich region. In some embodiments, the linker comprises a NLS sequence. In some embodiments, the linker comprises a NLS sequence flanked on each end by a glycine rich region.

In some embodiments, one or more of the at least one Cas protein and the at least one transposon-associated protein comprises a nuclear localization signal (NLS). In some embodiments, one or more of the at least one Cas protein and the at least one transposon-associated protein comprises two or more NLSs. In some embodiments, the NLS is appended to the one or more of the at least one Cas protein and the at least one transposon-associated protein at a N-terminus, a C-terminus, or a combination thereof.

The NLS may be a monopartite sequence or a bipartite sequence. In some embodiments, the NLS comprises a sequence having at least 70% similarity to KRTADGSEFESPKKKRKV (SEQ ID NO:4).

In some embodiments, the one or more nucleic acids comprises one or more messenger RNAs, one or more vectors, or a combination thereof.

In some embodiments, the at least one Cas protein, the at least one transposon-associated protein, and the gRNA are encoded by different nucleic acids.

In some embodiments, one or more of the at least one Cas protein, the at least one transposon-associated protein, and the gRNA are encoded by a single nucleic acid.

In certain embodiments, Cas7 is encoded by an individual nucleic acid. In certain embodiments, Cas7 or the nucleic acid encoding Cas7 is in greater abundance compared to the remaining protein components or nucleic acids encoding thereof.

In some embodiments, a single nucleic acid encodes the gRNA and at least one Cas protein (e.g., Cas6 or Cas7).

In some embodiments, each of the at least one Cas protein, the at least one transposon-associated protein, and the gRNA are encoded by a single nucleic acid.

In some embodiments, the at least one gRNA is a non-naturally occurring gRNA. In some embodiments, the at least one gRNA is encoded in a CRISPR RNA (crRNA) array. In some embodiments, the at least one gRNA is transcribed under control of an RNA Polymerase II or an RNA Polymerase III promoter.

In some embodiments, the one or more nucleic acids further comprises or encodes a sequence capable of forming a triple helix downstream of the sequence encoding the at least one Cas protein or the sequence encoding the at least one transposon-associated protein. In some embodiments, the sequence capable of forming a triple helix is in a 3′ untranslated region of the sequence encoding the at least one Cas protein or the sequence encoding the at least one transposon-associated protein.

In some embodiments, one or more of the nucleic acids encoding at least one Cas protein and the nucleic acids encoding the at least one transposon-associated protein comprises a sequence encoding a ribosome skipping peptide. In some embodiments, the ribosome skipping peptide comprises a 2A family peptide.

In some embodiments, the engineered CRISPR-Cas system is derived from Vibrio cholerae, Photobacterium iliopiscarium, Vibrio parahaemolyticus, Pseudoalteromonas sp., Pseudoalteromonas ruthenica, Photobacterium ganghwense, Shewanella sp., Vibrio diazotrophicus, Vibrio sp. 16, Vibrio sp. F12, Vibrio splendidus, Aliivibrio wodanis, Aliivibrio sp., Endozoicomonas ascidiicola, Parashewanella spongiae or Scytonema hofmannii.

Also provided are cells comprising the disclosed systems. In some embodiments, the cell is a prokaryotic cell. In some embodiments, the cell is a eukaryotic cell (e.g., a mammalian cell or a human cell).

Further disclosed are methods for recruiting one or more effector domains to a target nucleic acid in a cell and methods for modulating expression of a target gene in a cell introducing into a cell a system or a composition disclosed herein.

In some embodiments, the target nucleic acid sequence comprises the promoter region or the upstream activator sequence of the target gene.

In some embodiments, the cell is a prokaryotic cell. In some embodiments, the cell is a eukaryotic cell (e.g., a mammalian cell or a human cell).

In some embodiments, introducing the system into the cell comprises administering the system to a subject. In some embodiments, administering comprises in vivo administration. In some embodiments, administering comprises transplantation of ex vivo treated cells comprising the system.

Kits comprising any or all of the components of the systems described herein are also provided. In some embodiments, the kit further comprises one or more reagent, shipping and/or packaging containers, one or more buffers, a delivery device, instructions, or a combination thereof.

Other aspects and embodiments of the disclosure will be apparent in light of the following detailed description.

### BRIEF DESCRIPTION OF THE DRAWINGS

FIGS. 1A and 1B show the development of TniQ-Cascade-based transcriptional repressors. FIG. 1A is a schematic of transcriptional repression design. Binding of the Gal4-VP16 polypeptide to the Upstream Activator Sequence (UAS) leads to RNA Polymerase II recruitment, driving eYFP transcription. Co-transfection of QCascade and proper gRNAs leads to competitive inhibition of eYFP transcription, either by blocking Gal4-VP16 recruitment through competitive binding (gRNA-3) or by blocking RNA Pol II recruitment directly through competitive binding to the minimal CMV promoter (gRNA-1 and gRNA-2). In both cases, successful binding leads to decreased eYFP transcription and expression. FIG. 1B is a graph of eYFP mean fluorescence intensity (MFI) was determined upon co-transfection of the reporter plasmid and various TniQ-Cascade expression vector components, with or without various NLS (e.g., bipartite-NLS, or BP; SV40-NLS, or SV40) fusions. In some control experiments, protein components were intentionally omitted. The numbers above each bar within FIG. 1B represent experimental identifiers that correspond to the information described in Table 1.

FIGS. 2A-2E show the development of TniQ-Cascade-based transcriptional activators. FIG. 2A is an exemplary plasmid design for representative Cas8- or Cas7-activator constructs within a pcDNA3.I-derivative expression vector. One of various activator domains may be fused to either the N- or C-terminus (or both) of one or more Cas8 or Tns proteins. FIG. 2B is a schematic of the transcriptional activation assay. A reporter plasmid contains a minimal CMV promoter, an mCherry cassette, and an upstream 32 base pair target sequence with a 5′ CC PAM. When transfected alone, the reporter minimally expresses mCherry. When plasmids expressing QCascade protein components with an activator fused to either Cas8 or Cas7, together with a plasmid encoding a gRNA that targets the reporter plasmid, are co-transfected, QCascade can be expressed, complexed, and then target the reporter plasmid target sequence, leading to transcriptional activation, RNA Pol II recruitment, and thus elevated levels of mCherry expression, which are quantified by flow cytometry. FIG. 2C is a graph of the E. coli integration efficiency of VchINTEGRATE with various activator fusions to Cas8 and Cas7. RPV refers to a tripartite activator fusion construct comprising Rta-P65-VP64. These results demonstrate that VP64 and RPV fusions to the N-terminus of Cas7 do not negatively affect DNA integration activity. Integration efficiencies are shown for both tRL and tLR orientation products. FIG. 2D is a bar graph showing mCherry MFI relative to the non-targeting (NT) control (bar 69) for transcriptional activation experiments with various QCascade constructs. Activation is achieved with a targeting (T) gRNA and VP64-Cas7 fusion protein. SV40 refers to SV40 NLS tags. FIG. 2E is a bar graph showing mCherry MFI relative to the non-targeting (NT) gRNA for transcriptional activation experiments with various CRISPR systems, and with various tagged version of QCascade protein components. Notably, transcriptional activation with the VchINTEGRATE QCascade complex is noticeably higher when all protein components contain a bipartite-NLS (BP) tag, as opposed to a monopartite SV40 NLS tag (compare bars 78-80). Activation is eliminated in control experiments with a non-targeting gRNA or when Cas6 or Cas8 are removed. Comparisons are shown to S. pyogenes dCas9 (Type II-A) and Pseudomonas S-6-2 Cascade (Type I-E). The numbers above each bar within FIGS. 2C-2E represent experimental identifiers that correspond to the information described in Table 1.

FIGS. 3A-3E show TnsC forms an oligomeric complex that is specifically recruited to DNA by TniQ-Cascade. FIG. 3A (bottom) is chromatograms of size exclusion chromatography (SEC) on a Superdex 200 column of VchTnsC (from Tn6677; VchINTEGRATE) forming a large MW species in the presence of ATP; comparison to standards (right) indicates a MW consistent with a heptamer (predicted molecular weight. MW=268 kDa). FIG. 3A (top) is an SDS-PAGE gel above indicating the protein fractions from the SEC run containing ATP in the reaction. FIG. 3B is raw cryo-electron microscopy (cryoEM) micrographs (right) and reference-free 2D class averages of TnsC•ATP (left) reveal 7-spoked rings with a central pore. FIG. 3C is orthogonal views of a preliminary 3D reconstruction of the TnsC•ATP heptamer, with dimensions shown below. The central pore perfectly accommodates the dimensions of modeled double-stranded DNA (dsDNA, right). FIG. 3D is CryoEM data with TnsC•ATP in low salt shows clear presence of larger filament assemblies. FIG. 3E shows that VchTnsC binds genomic target sites in E. coli with extremely high genome-wide specificity, as assayed by chromatin immunoprecipitation followed by next-generation sequencing (ChIP-seq). Cells expressed a gRNA targeting the region shown with a red triangle (target-4, found in lacZ) alongside all protein components from VchINTEGRATE and the donor DNA. Either VchCas8 (top) or VchTnsC (bottom) contained a 3×FLAG tag. Cells were crosslinked, lysates were sonicated to shear DNA, and protein-DNA crosslinked species were isolated by ChIP, followed by NGS library prep and deep sequencing. These experiments show remarkably high-specificity target binding by TnsC, in a mechanism that depends on the presence of a targeting gRNA and all TniQ-Cascade components.

FIGS. 4A-4D show TnsC-activator fusion proteins can be combined with TniQ-Cascade for potent, high-efficiency synthetic transcriptional activation. FIG. 4A is exemplary plasmid design for TnsC-VP64 constructs within a pcDNA3.1-derivative expression vector. VchTnsC derived from Tn6677 (VchINTEGRATE) is fused to a NLS tag and Vp64 domain. FIG. 4B is a schematic of the transcriptional activation assay. When transfected alone, the mCherry reporter as described in FIG. 2 minimally expresses mCherry because it is controlled by a minimal CMV promoter. When plasmids expressing QCascade, TnsC-VP64, and a gRNA that recognizes the target present on the reporter plasmid are co-transfected, QCascade (blue oval) binds to the target sequence and recruits TnsC-VP64 (light orange ovals), leading to elevated levels of mCherry expression. Three copies of TnsC-VP64 are shown for simplicity to demonstrate the oligomeric nature of TnsC recruitment; the actual number of TnsC proteins that are recruited to target sites in cells may be significantly larger. FIG. 4C is a bar graph showing mCherry activation with various CRISPR-Cas components systems, and with various tagged version of TniQ-Cascade and TnsC components. Data were measured by flow cytometry, and the cellular mCherry mean fluorescence intensity (MFI) was plotted relative to the non-targeting gRNA control for each system, indicated at the bottom. dCas9 derives from S. pyogenes; “Psp S-6-2” refers to a Type I-E Cascade from a Pseudomonas species; Vch refers to VchINTEGRATE derived from Tn6677. As seen on the right, the presence of TnsC fused to BP and VP64, when combined with BP-tagged QCascade components, leads to potent transcriptional activation. This activation is lost when the VP64 domain is removed (bar 94), and when TniQ is omitted (bar 95). Additional permutations are shown and labeled in the text below the bar graph. FIG. 4D is a bar graph showing mCherry activation via TnsC-VP64 fusion proteins, measured in the presence of TnsA, TnsB, or TnsAB_{f}. Data are presented as in FIG. 4C and are plotted relative to the non-targeting (NT) control. TnsAB_{f} together with TniQ-Cascade and the TnsC-BP-VP64 activator led to heightened levels of transcriptional activation. The numbers above each bar within FIGS. 4C and 4D represent experimental identifiers that correspond to the information described in Table 1.

FIGS. 5A and 5B show VchCas7 variants for nuclear trafficking and QCascade assembly and activity. FIG. 5A is schematics of various exemplary constructs generated and tested within a pcDNA3.1-derivative expression vector. VchCas7 (derived from VchINTEGRATE) is cloned downstream of a CMV promoter, with inclusion of either one or more SV40 or BP NLS tags, with inclusion of an optional 3×FLAG tag. FIG. 5B is a bar graph showing QCascade and TnsC transcriptional activation data using different Cas7 variants described in FIG. 5A. TnsC activation experiments were performed and analyzed as described in FIG. 4 and are normalized to the non-targeting gRNA data in bar number 104. The numbers above each bar within FIG. 5B represent experimental identifiers that correspond to the information described in Table 1.

FIG. 6 is a bar graph showing TnsC-mediated transcriptional activation data using different ratios of QCascade and gRNA expression plasmids relative to the TnsC-VP64 expression plasmid. TnsC-VP64 activation experiments were designed and performed as described in FIG. 4, and cellular mCherry MFI was measured by flow cytometry and plotted relative to the non-targeting (NT). Plasmid amounts are shown relative to the amount of TnsC-VP64 plasmid for each transfection, and data are compared to the level of transcriptional activation for S. pyogenes dCas9 fused to VPR. These data show that the level of QCascade and TnsC activation exceeds that of dCas9-VPR when the amount of Cas8 expression plasmid is increased, indicating that this factor was limiting in other experiments. The numbers above each bar represent experimental identifiers that correspond to the information described in Table 1.

FIGS. 7A-7J are schematics of exemplary methods for utilizing QCascade and TnsC for transcriptional repression, transcriptional activation, epigenome editing, base editing, chromosomal DNA imaging, and combinatorial effector delivery. FIG. 7A is a schematic of a possible mechanism of RNA-guided target DNA binding by the TniQ-Cascade (aka QCascade) complex and subsequent recruitment of the AAA+ ATPase, TnsC. The PAM is shown in yellow and target DNA strand is shown in maroon, in the top panel. Target DNA binding involves formation of an R-loop, and the QCascade complex comprises the gRNA (aka crRNA), one copy of Cas8, 6 copies of Cas7, 1 copy of Cas6, and 2 copies of TniQ. The spacer sequence of the gRNA is 32-nucleotides long, though alternate length spacers may also be used, and retain RNA-guided DNA targeting and integration ((Klompe et al., Nature 571, 219-225 (2019); Songailiene, I. et al. Cell Rep 28, 3157-3166.e4 (2019)). QCascade forms a structural scaffold on target DNA that leads to recruitment of multiple copies of TnsC, which subsequently leads to recruitment of the donor DNA bound by TnsA and TnsB, and eventual targeted integration (triangle). Note that the exact number of TnsC subunits that get recruited is not known but is much greater than 1. The representative pathway is shown for a Type I-F CRISPR-transposon system (e.g., VchINTEGRATE), and is also applicable for the I-B system, where the Cas8 protein (a natural Cas8-Cas5 fusion for I-F) is instead joined by a separate molecule of Cas5. The pathway in Type V-K CRISPR-transposon systems is similar, but R-loop formation is driven by binding of Cas12k and a dual-guide RNA (or engineered single-guide RNA, sgRNA). FIG. 7B shows one or more of the N- and/or C termini of the different protein subunits comprising TniQ-Cascade and TnsC may be fused to desired effector domains, for the generation of novel genome perturbation reagents. In various embodiments, these may include transcriptional activators; transcriptional repressors; epigenome modification reagents; DNA methylation or deamination reagents; fluorescent proteins; alternative scaffolding proteins; histone modification enzymes; nuclease enzymes; and more. Nuclear localization signals (NLS) are also included for effector fusion designs, in order to direct efficient nuclear trafficking, but are not shown. In other embodiments. Cascade components traffic to the nucleus after the entire complex forms in the cytoplasm, such that not all protein components contain their own NLS. FIG. 7C is a schematic of transcriptional activators built around the Cascade complex and the fusion of one or more transcriptional activation domains to either the N- or C-terminus, or both termini, of Cas7. Representative domain schematics of various activator constructs are shown at top. Because Cas7 is present in multiple copies in the DNA-bound Cascade complex (6 copies, for a 32-nucleotide spacer), the activation domains are displayed with high valency at the target site, leading to robust transcriptional activation. These Cascade activators may include TniQ (bottom right) or may use only the Cas8-Cas7-Cas6 protein components (bottom left), absent TniQ. Nuclear localization signals (NLS) are also included for effector fusion designs, in order to direct efficient nuclear trafficking, but are not shown. In other embodiments. Cascade components traffic to the nucleus after the entire complex forms in the cytoplasm, such that not all protein components contain their own NLS. FIG. 7D is a schematic of transcriptional activators built around the AAA+ ATPase by fusing activation domains to the C-terminus of TnsC. Various activation domains may be used in distinct embodiments, or multiple effector domains may be fused together. Because TnsC is recruited to DNA via the target-bound QCascade complex, in a TniQ-dependent fashion, and is recruited in multiple copies, the activation domains are displayed with high valency at the target site, leading to robust transcriptional activation. In other embodiments, similar TnsC fusions are constructed using TnsC from Type V-K CRISPR-transposon systems, whereby target DNA is mediated by Cas12k and gRNA, leading to a similar TniQ-dependent TnsC recruitment pathway. Nuclear localization signals (NLS) are also included for effector fusion designs, in order to direct efficient nuclear trafficking, but are not shown. FIG. 7E is a schematic of transcriptional repressors constructed by fusing repression domains either to Cas8, Cas7, or Cas6 (not shown), or to the C-terminus of TnsC (shown). The repression domain may be a KR AB domain, or other histone modification domains such as histone methylases, or other DNA modification domains, such as DNA methyltransferases. The multi-valent recruitment of TnsC to the QCascade-bound target DNA leads to potent repression of gene regulation. Nuclear localization signals (NLS) are also included for effector fusion designs, in order to direct efficient nuclear trafficking, but are not shown. FIG. 7B is a schematic of QCascade and TnsC for high signal-to-noise chromosomal locus imaging, by fusing either Cas7 (not shown) or TnsC (shown) to GFP or another fluorescent protein (FP). Because both Cas7 and TnsC are recruited to the target DNA in multiple copies, a single gRNA leads to recruitment of multiple copies of the FP fusion protein, leading to high fluorescence signal at the target site. Nuclear localization signals (NLS) are also included for effector fusion designs, in order to direct efficient nuclear trafficking, but are not shown. FIG. 7G is a schematic of the construction of base editors by fusing either cytidine deaminase (Cyt. deaminase; Cyt. deam.) or adenine deaminase (2×TadA) domains to either the Cas8 or Cas7 subunits of Cascade. The cytidine deaminase domain is combined with two tandem uracil glycosylase inhibitor domains, as described previously (Doman, J. L. et al. Nat Biotechnol 38, 620-628 (2020); and references therein). The fusions to Cas8 or Cas7 may occur in multiple different arrangements, and lead to variable windows of base editing activity, which may be applied for various desired editing outcomes. The 2×TadA effector may be fused to either the N- or C-terminus of Cas7. These editing reagents may be used together with all components of the QCascade complex, or only the components of the Cascade complex (e.g., omitting TniQ). Nuclear localization signals (NLS) are also included for effector fusion designs, in order to direct efficient nuclear trafficking, but are not shown. In other embodiments, Cascade components traffic to the nucleus after the entire complex forms in the cytoplasm, such that not all protein components must contain their own NLS. FIG. 7H is a schematic of potent transcriptional activators generated through combinatorial addition of effector domains to both the TnsC and Cas7 subunits, simultaneously. Four embodiments are shown at left, in which TnsC and Cas7 are fused to the effector domains indicated. This architecture, when combined with Cas6, Cas8, TniQ, and the gRNA, leads to targeted recruitment of multiple distinct effector domains to a single target site specified by the gRNA (right), leading to high-activity synergistic transcriptional activation. In other embodiments, other effector domain combinations are tested, including effector fusions to the C-terminus of Cas7 instead of the N-terminus. Nuclear localization signals (NLS) are also included for effector fusion designs, in order to direct efficient nuclear trafficking, but are not shown. In other embodiments, Cascade components traffic to the nucleus after the entire complex forms in the cytoplasm, such that not all protein components must contain their own NLS. FIG. 7I is a schematic of potent transcriptional repressors and epigenome editors generated by the combinatorial fusion of multiple effector domains to both TnsC and Cas7. In one embodiment, these comprise the KRAB and Dnmt3A/3L domains used in CRISPRoff reagents (Nuñez, J. K. et al. Cell 184, 2503-2519.e17 (2021)). In other embodiments, additional effector domains are used. The multi-valent recruitment of both Cas7 and TnsC to the target site, guided by a single gRNA, leads to potent and durable recruitment of the associated repressor or epigenome modification domains. Nuclear localization signals (NLS) are also included for effector fusion designs, in order to direct efficient nuclear trafficking, but are not shown. In other embodiments. Cascade components traffic to the nucleus after the entire complex forms in the cytoplasm, such that not all protein components contain their own NLS. FIG. 7J is a schematic of high signal-to-noise DNA labeling/imaging reagents constructed by the combinatorial fusion of multiple spectrally distinct fluorescent proteins (FPs) to both TnsC and Cas7; GFP and RFP fusions are shown in the provided examples (left and right). Because a single gRNA localizes multiple copies of both proteins to the same genomic target site, the dual-color labeling leads to higher signal-to-noise as compared to imaging reagents with just a single color, allowing for internal verification of labeling specificity. In other embodiments, FRET pairs are used, such that the proteins transfer energy and allow for even greater discrimination between co-localized TnsC and Cas7 proteins at the target site, relative to freely diffusing molecules in the cytoplasm and nucleus, thereby reducing the background. Nuclear localization signals (NLS) are also included for effector fusion designs, in order to direct efficient nuclear trafficking, but are not shown. In other embodiments, Cascade components traffic to the nucleus after the entire complex forms in the cytoplasm, such that not all protein components must contain their own NLS.

FIGS. 8A and 8B show that modified gRNA expression cassettes retain potent RNA-guided DNA targeting activity. FIG. 8A is schematic of an exemplary initial gRNA expression strategy (top) employing a separate plasmid encoding the gRNA as a repeat-spacer-repeat array, controlled by a human U6 promoter, and a modified pDonor plasmid (bottom) in which the CRISPR array expression cassette is placed just downstream of the mini-transposon. FIG. 8B is a graph of QCascade and TnsC-VP64 transcriptional activation using the modified gRNA expression plasmids, in which the gRNA was encoded on pDonor itself. The levels of activation, as measured by relative mCherry MFI (normalized to the non-targeting control) are nearly indistinguishable between the initial gRNA expression strategy (FIG. 8A, top) and the modified strategy in which the gRNA is encoded on pDonor (FIG. 8A, bottom). The numbers above each bar in FIG. 8B represent experimental identifiers that correspond to the information described in Table 1.

FIGS. 9A-9C show RNA Polymerase II-based expression of guide RNAs for VchINTEGRATE. FIG. 9A is schematics of different methods to express the gRNA. The CRISPR array (repeat-spacer-repeat) is canonically encoded on an RNA Pol III promoter (e.g., human U6), such that the nascent transcript stays primarily nuclear. However, it can also be encoded within the 3′-UTR of an RNA Pol II transcript, alongside the use of features such as the MALAT1 triplex to stabilize upstream protein-coding transcripts after cleavage. Cleavage occurs upon repeat-spacer-repeat processing by the Cas6 ribonuclease subunit of Cascade. FIG. 9B is schematic of the various constructs generated and tested within a pcDNA3.1-derivative expression vector. The MALAT1 triplex and CRISPR array were inserted into the 3′-UTR of either VchCas6 or VchCas7. FIG. 8C is a bar graph showing transcriptional activation data using constructs described in FIG. 9B. These results demonstrate that Pol II-encoded gRNAs are functional for RNA-guided DNA targeting and TnsC-based activation above background, defined here as the non-targeting gRNA control. The numbers above each bar in FIG. 9C represent experimental identifiers that correspond to the information described in Table 1.

FIG. 10A is an exemplary schematic for TnsC-VP64 transcriptional activation. FIG. 10B is graph of the normalized activation as measured by relative gene expression of TTN via quantitative reverse transcription PCR (RT-qPCR). FIG. 10C is a schematic of four exemplary individual crRNA expression plasmids, or one plasmid expressing an unprocessed CRISPR array containing four spacer sequences targeting the same region as the individual crRNA plasmids. FIG. 10D is schematics of crRNA expression plasmids targeting endogenous genes TIN, MIAT, ASCL1, and ACTC1. FIG. 10E is a graph of normalized activation observed for three additional genomic loci, MIAT, ASCL1, and ACTC1.

### DETAILED DESCRIPTION

The disclosed systems, kits, and methods provide systems and methods for RNA-guided recruitment of effector molecules (e.g., activators, repressors, nucleic acid modifiers) to DNA.

Provided herein are systems and methods that provide synthetic effectors which mediate protein activity through nucleic acid binding in a guide-RNA dependent manner. The oligomeric properties of the system allow downstream applications with more potency and dynamic range than would be possible with a single-copy proteins and systems like dCas9. Furthermore, the multi-component nature of the disclosed systems allows for combinatorial recruitment of multiple different types of effector domains to a single location for synergistic or tunable activity. Thus, the disclosed systems can lead to tunable, potent signal amplification.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009). Durbin et al., eds., Biological Sequence Analysis: Probabilistic Models of Proteins and Nucleic Acids, Cambridge University Press, Cambridge. UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. Hybridization methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA, 46: 453 (1960) and Doty et al., Proc. Natl. Acad. Sci. USA, 46: 461 (1960), have been followed by the refinement of this process into an essential tool of modern biology. For example, hybridization and washing conditions are now well known and exemplified in Sambrook et al., supra. The conditions of temperature and ionic strength determine the “stringency” of the hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) may also be considered a “double-stranded nucleic acid.” For example, triplex structures are considered to be “double-stranded.” In some embodiments, any base-paired nucleic acid is a “double-stranded nucleic acid.”

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement of the systems of the disclosure into a cell, organism, or subject by a method or route which results in at least partial localization of the system to a desired site. The systems can be administered by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### CRISPR-Cas Systems for DNA Integration

In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Transcription of a CRISPR locus produces a “pre-crRNA,” which is processed to yield crRNAs containing spacer-repeat fragments that guide effector nuclease complexes to cleave dsDNA sequences complementary to the spacer. Several different types of CRISPR systems are known, (e.g., type 1, type 11, or type III), and classified based on the Cas protein type and the use of a proto-spacer-adjacent motif (PAM) for selection of proto-spacers in invading DNA.

Although RNA-guided targeting typically leads to endonucleolytic cleavage of the bound substrate, recent studies have uncovered a range of noncanonical pathways in which CRISPR protein-RNA effector complexes have been naturally repurposed for alternative functions.

Disclosed herein are systems or kits for effector domain recruitment to a target nucleic acid sequence comprising: an engineered Clustered Regularly Interspaced Short Palindromic Repeats (CRISPR)-CRISPR associated (Cas) system or one or more nucleic acids encoding the engineered CRISPR-Cas system, wherein the CRISPR-Cas system comprises at least one or both of: a) at least one Cas protein; and b) a guide RNA (gRNA) complementary to at least a portion of the target nucleic acid sequence, wherein one or more of the at least one Cas protein comprises at least one effector domain. In some embodiments, the target nucleic acid comprises a promoter region of a gene of interest. In some embodiments, the target nucleic acid comprises an upstream activator sequence. In some embodiments, the gene of interest is located on a chromosome in a cell.

Also disclosed herein are systems or kits for effector domain recruitment to a target nucleic acid sequence comprising: an engineered Clustered Regularly Interspaced Short Palindromic Repeats (CRISPR)-CRISPR associated (Cas) system or one or more nucleic acids encoding the engineered CRISPR-Cas system, wherein the CRISPR-Cas system comprises at least one or both of: a) at least one Cas protein; b) at least one transposon-associated protein; and c) a guide RNA (gRNA) complementary to at least a portion of the target nucleic acid sequence, wherein one or more of the at least one Cas protein and the at least one transposon-associated protein comprises at least one effector domain.

In some embodiments, one or more of the at least one Cas protein are part of a ribonucleoprotein complex with the gRNA. In some embodiments, the ribonucleoprotein complex comprises one or more of the at least one transposon-associated protein.

In some embodiments, the system comprises two or more engineered CRISPR-Cas systems. Pairing of orthogonal systems allows tandem recruitment of multiple distinct effectors to different target nucleic acids. For example, one, two, three, four, five, or more orthogonal CRISPR-Cas systems may be used to deliver multiple effectors to various target nucleic acids.

The CRISPR-Cas system(s) may be derived from Vibrio cholerae, Photobacterium iliopiscarium, Vibrio parahaemolyticus, Pseudoalteromonas sp., Pseudoalteromonas ruthenica, Photobacterium ganghwense, Shewanella sp., Vibrio diazotrophicus, Vibrio sp. 16, Vibrio sp. F12, Vibrio splendidus, Aliivibrio wodanis, Aliivibrio sp., Endozoicomonas ascidiicola, Parashewanella spongiae or Scytonema hofmannii.

Also disclosed is a cell comprising the system described herein. In some embodiments, the cell is a prokaryotic cell. In some embodiments, the cell is a eukaryotic cell. In some embodiments, the cell is a mammalian cell (e.g., a human cell).

a. CRISPR-Cas System

CRISPR-Cas systems are currently grouped into two classes (1-2), six types (I-VI) and dozens of subtypes, depending on the signature and accessory genes that accompany the CRISPR array. The engineered CRISPR-Cas system may be derived from a Class I CRISPR-Cas system or a Class 2 CRISPR-Cas system. The present system may be derived from a Type I CRISPR-Cas system (such as subtypes I-B, I-D, I-F (including I-F variants). The present system may be derived from a Type V CRISPR-Cas system.

Type I CRISPR-Cas systems encode a multi-subunit protein-RNA complex called Cascade, which utilizes a crRNA (or guide RNA) to target double-stranded DNA during an immune response. Cascade itself has no nuclease activity, and degradation of targeted DNA is instead mediated by a trans-acting nuclease known as Cas3. In Type I-A and I-D systems, the activities of Cas3 are carried out by separate proteins called Cas3′ (helicase) and Cas3″ (nuclease). Type I-D systems also comprise Cas10d instead of Cas8.

In some embodiments, the engineered CRISPR-Cas system comprises Cas5, Cas6, Cas7, Cas8, or any combination thereof. In some embodiments, the engineered CRISPR-Cas system comprises a Cas8-Cas5 fusion protein. In some embodiments, the engineered CRISPR-Cas system comprises Cas5, Cas6, Cas7, and Cas8. In some embodiments, the engineered CRISPR-Cas system comprises Cas5, Cas6, Cas7, and Cas10d.

Type V systems belong to the Class 2 CRISPR-Cas systems, characterized by a single-protein effector complex that is programmed with a gRNA. The transposon-associated Type V CRISPR-Cas systems may be derived from: Anabaena variabilis ATCC 29413 (or Trichormus variabilis ATCC 29413 (see GenBank CP(00117.1)), Cyanobacterium aponinum IPPAS B-1202. Filamentous cyanobacterium CCP2, Nostoc punctiforme PCC 73102, and Scytonema hofmannii PCC 7110.

In some embodiments, the engineered CRISPR-Cas system comprises Cas12k, previously known as C2c5.

A system of the present invention may comprise at least one transposon-associated protein (e.g., transposases or other components of a transposon), or a nucleic acid encoding thereof. The transposon-associated proteins may facilitate recognition of the target nucleic acid.

In some embodiments, the transposon-associated proteins are derived from a Tn7 or Tn7-like transposon. Tn7 and Tn7-like transposons may be categorized based on the presence of the hallmark DDE-like transposase gene, tnsB (also referred to as tniA), the presence of a gene encoding a protein within the AAA+ ATPase family, tnsC (also referred to as tniB), one or more targeting factors that define integration sites (which may include a protein within the tniQ family, also referred to as tnsD, but sometimes includes other distinct targeting factors), and inverted repeat transposon ends that typically comprise multiple binding sites thought to be specifically recognized by the TnsB transposase protein. In Tn7, the targeting factors, or “target selectors,” comprise the genes InsD and insE. Based on biochemical and genetics studies, it is known that TnsD binds a conserved attachment site in the 3′ end of the glmS gene, directing downstream integration, whereas TnsE binds the lagging strand replication fork and directs sequence-non-specific integration primarily into replicating/mobile plasmids.

The most well-studied member of this family of transposons is Tn7, hence why the broader family of transposons may be referred to as Tn7-like. “Tn7-like” term does not imply any particular evolutionary relationship between Tn7 and related transposons; in some cases, a Tn7-like transposon will be even more basal in the phylogenetic tree and thus Tn7 can be considered as having evolved from, or derived from, this related Tn7-like transposon.

Whereas Tn7 comprises tnsD and tnsE target selectors, related transposons comprise other genes for targeting. For example, Tn5090/Tn5053 encode a member of the tniQ family (a homolog of E. coli tnsD) as well as a resolvase gene tniR; Tn6230 encodes the protein TnsF; and Tn6022 encodes two uncharacterized open reading frames orf2 and orf3; Tn6677 and related transposons encode variant Type I-F and Type I-B CRISPR-Cas systems that work together with TniQ for RNA-guided mobilization; and other transposons encode Type V-U5 CRISPR-Cas systems that work together with TniQ for random and RNA-guided mobilization. Any of the above transposon systems are compatible with the systems and methods described herein.

In some embodiments, the at least one transposon-associated protein comprises TniQ. In some embodiments, the at least one transposon-associated protein further comprises TnsC.

In some embodiments, the at least one transposon-associated protein further comprises TnsA and TnsB, also known as TniA and TniB. In some embodiments, the at least one transposon protein comprises a TnsA-TnsB fusion protein. TnsA and TnsB can be fused in any orientation: N-terminus to C-terminus; C-terminus to N-terminus; N-terminus to N-terminus; or C-terminus to C-terminus, respectively. Preferably the C-terminus of TnsA is fused to the N-terminus of TnsB.

In some embodiments, the TnsA-TnsB fusion may be fused using an amino acid linker peptide of various lengths to provide greater physical separation and allow more spatial mobility between the fused portions. The linker may comprise any amino acids and may be of any length. The linker may be less than about 50 (e.g., 40, 30, 20, 10, or 5) amino acid residues.

In some embodiments, the linker is a flexible linker, such that TnsA and TnsB can have orientation freedom in relationship to each other. For example, a flexible linker may include amino acids having relatively small side chains, and which may be hydrophilic. Without limitation, the flexible linker may contain a stretch of glycine and/or serine residues. In some embodiments, the linker comprises at least one glycine-rich region. For example, the glycine-rich region may comprise a sequence comprising [GS]n, wherein n is an integer between 1 and 10.

In some embodiments, the linker further comprises a nuclear localization sequence (NLS). The NLS may be embedded within a linker sequence, such that it is flanked by additional amino acids. In some embodiments, the NLS is flanked on each end by at least a portion of a flexible linker. In some embodiments, the NLS is flanked on each end by a glycine rich region of the linker. Suitable nuclear localization sequences for use with the disclosed system are described further below and are applicable to use with the TnsA-TnsB fusion protein. In some embodiments, the linker comprises the amino acid sequence of GCGCGKRTADGSEFESPKKKRKVGSGSGG (SEQ ID NO: 1).

In certain embodiments, the TnsA-TnsB fusion protein comprises an amino acid sequence having at least 70% (at least 75%, at least 80%, at least 85%, at least 90%, at least 95%) similarity to that of SEQ ID NOs: 9-14. For example, the TnsA-TnsB fusion protein may comprise an amino acid sequence having one or more (e.g., 1, 2, 3, 4, 5, 6, 7, 8, 9, 10, 12, 15, 18, or 20) substitutions compared to that of SEQ ID NOs: 9-14.

In some embodiments, any combination of the at least one Cas protein and the at least one transposon associate protein may be expressed as a single fusion protein. For example, in some embodiments, each of the at least one Cas protein are part of a single fusion protein. In some embodiments, each of the at least one Cas protein and one or more of the at least one transposon-associated protein are part of a single fusion protein in which the components are expressed as a single megapeptide.

Sequences of exemplary Cas proteins and transposon-associated proteins can be found in International Patent Publication WO2020181264 and International Patent Application PCT/US22/32541, each incorporated herein by reference.

However, the invention is not limited to the disclosed or referenced exemplary sequences. Indeed, genetic sequences can vary between different strains, and this natural scope of allelic variation is included within the scope of the invention.

In other embodiments, any of the proteins described or referenced herein may comprise a sequence corresponding to, or substantially corresponding to, the wild-type version of the protein. For example, the sequence may substantially correspond to the wild-type protein sequence except for changes made for facile cloning or removal of known restriction sites. Thus, protein products from potential alternative start codons compared to the predicted nucleic acid sequences in this document are therefore not excluded.

Any of the proteins described or referenced herein may comprise one or more amino acid substitutions as compared to the recited sequences. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer. Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

The components of the system may be present in the system in various ratios. In some embodiments, each of the protein components or the nucleic acids encoding thereof are provided in a 1:1 ratio. For example, when each protein component is encoded on a single nucleic acid, the single nucleic acid comprises a single coding sequence for each protein component.

In some embodiments, any one of the protein components may be provided in greater abundance to any other protein component. In certain embodiments. Cas7 or the nucleic acid encoding Cas7 in greater abundance compared to the remaining protein components or nucleic acids encoding thereof. For example, multiple copies of a nucleic acid encoding Cas7 may be provided for each copy of any of the other components (e.g., Cas6, Cas5, Cas8, TniQ or TnsC). In some embodiments, Cas7 is encoded on a nucleic acid separate from any of the other components such that it can be provided in the system and methods herein at a higher abundance or dosage than the other components. Analogously, higher concentrations of the Cas7 protein can be provided in the systems and methods compared to the other proteins. In some embodiments, for every one copy of Cas6 or Cas8, or nucleic acids encoding thereof, 2 or more copies of Cas7 or a nucleic acid encoding Cas7 are included in the system. In some embodiments, for every one copy of Cas6 or Cas8 or nucleic acids encoding thereof, 5-10 copies of Cas7 or a nucleic acid encoding Cas7 are included in the system.

a. Effector Domain(s)

In the systems disclosed herein, one or more of the at least one Cas protein and, when the system comprises at least one transposon-associated protein, the at least one transposon-associated protein may comprise at least one effector domain. The at least one effector domain may be appended to one or more of the at least one Cas protein and the at least one transposon-associated protein at a N-terminus, a C-terminus, internally, or a combination thereof. The effector domains may be fused in any orientation in relationship to the at least one Cas protein and the at least one transposon-associated protein.

In some embodiments, one or more of the at least one Cas protein and the at least one transposon-associated protein may comprise two or more effector domains. The effector domains may be fused to the at least one Cas protein and the at least one transposon-associated protein in tandem or individually, for example, at the N-terminus and at the C-terminus.

Effector domains contain any protein or fragments thereof that can modify, regulate, or tag a target nucleic acid. The effector domain may comprise a number of functionalities, including but not limited to, nuclease function, recombinase function, epigenetic modifying function, transposase function, integrase function, resolvase function, invertase function, protease function, DNA methyltransferase function, DNA demethylase function, histone acetylase function, histone deacetylase function, transcriptional repressor function, transcriptional activator function, DNA binding protein function, transcription factor recruiting protein function, nuclear-localization signal function, DNA editing function (e.g., deaminase) or any combination thereof. For example, some effector domains function in transcriptional regulation via their ability to interact with the basal transcriptional machinery and general coactivators, interact with other transcription factors to allow cooperative binding, and/or directly or indirectly recruit histone and chromatin modifying enzymes. In some embodiments, any additional domains or proteins necessary for the functionality of the effector domain may be provided as a fusion to the one or more of the at least one Cas protein and the at least one transposon-associated protein or separately.

In some embodiments, the system described herein is used to modulate gene regulatory activity, such as transcriptional or translational activity. For example, the at least one effector domain may comprise activator and/or repressor activity that can affect transcription upstream and downstream of coding regions, and can be used to activate or repress gene expression. In some embodiments, the at least one effector domain may include domains from transcription factors (activators, repressors, coactivators, co-repressors), silencers, and/or chromatin associated proteins and their modifiers (e.g., methylases, demethylases, acetylases and deacetylases).

Accordingly, in some embodiments, a system as disclosed herein having a transcription activator effector domain can be used to directly increase gene expression. In some embodiments, a system as disclosed herein comprising a transcriptional protein recruiting domain, or active fragment thereof, can be used to recruit transcriptional activators or repressors to a specific nucleic acid sequence to localize activators and repressors to modulate gene expression in a targeted manner.

In some embodiments, the at least one effector domains comprise transcriptional repressor function. Transcription repressors prevent, partially or completely, the transcription of genes near to its target site. Exemplary transcriptional repressors include, but are not limited to, KRAB-domain containing proteins, SID, and Spl.

In some embodiments, the at least one effector domains comprise transcriptional activator function. Transcriptional activators can be generally defined as proteins, or domains thereof, that bind to specific sites on promoter DNA and bring about increased transcription of specific genes through interactions with other proteins. Exemplary transcriptional activators include, but are not limited to, VP64, p65, p53, c-Myb, GATA-1, EKLF, MyoD, E2F, dTCF, Tat, HSF1, RTA and SET7/9.

In some embodiments, the at least one effector domains comprise DNA methyltransferase or DNA methylase function. DNA methyltransferases (DNMT's) are a family of DNA modifying proteins composed of different isomers (e.g., DNMT1, DNMT3A, and DNMT3B). Other exemplary DNA methyltransferases include SssI methylase, AluI methylase, HaeIII methylase, HhaI methylase, and HpaII methylase. Their main mechanism of action is addition of a methyl group to the fifth carbon of a cytosine residue (5mc) located adjacent to a guanine residue.

In some embodiments, the at least one effector domains comprise DNA demethylase function. DNA demethylation can be mediated by at least three enzyme families: (i) the ten-eleven translocation (TET) family, mediating the conversion of 5mC into 5hmC; (ii) the AID/APOBEC family, acting as mediators of 5mC or 5hmC deamination; and (iii) the BER (base excision repair) glycosylase family involved in DNA repair.

Kinases, phosphatases, and other proteins that modify or regulate other polypeptides involved in gene regulation are also useful as effector domains. Such modifiers are often involved in switching on or off transcription mediated by, for example, hormones. Other useful domains for regulating gene expression can also be obtained from the gene products of oncogenes (e.g., myc, jun, fos, myb, max, mad, rel, ets, bcl, myb, mos family members) and their associated factors and modifiers.

The at least one effector domains can be used to target enzymatic activity to locations containing the target nucleic acid sequence to which the gRNA is directed. For example, in some embodiments, effector domains having integrase or transposase activity can be used to promote integration of exogenous nucleic acid sequence into specific nucleic acid sequence regions and/or eliminate (knock-out) specific endogenous nucleic acid sequence.

Integrases allow for the insertion of nucleic acids, for example, into a host genome (mammalian, human, mouse, rat, monkey, frog, fish, plant (including crop plants and experimental plants like Arabidopsis), laboratory or biomedical cell lines or primary cell cultures, C. elegans, fly (Drosophila), etc.). Integrases are found in a retrovirus such as HIV (human immunodeficiency virus) and lambda integrase.

In some embodiments, the at least one effector domains comprise transposase functionality. Transposases are enzymes that bind to the end of a transposon and catalyze its movement by a cut and paste mechanism or a replicative transposition mechanism. Exemplary transpoases include, but are not limited to, Tcl transposase, Mos1 transposase, Tn5 transposase, and Mu transposase

In some embodiments, the at least one effector domains modify epigenetic signals and thereby modify gene regulation, for example by promoting histone acetylase and histone deacetylase activity. The term “epigenetic modifier.” as used herein, refers to a protein or catalytic domain thereof having enzymatic activity that results in the epigenetic modification of DNA, for example, chromosomal DNA. Epigenetic modifications include, but are not limited to, histone modifications including methylation and demethylation (e.g., mono-, di- and tri-methylation), histone acetylation and deacetylation, as well as histone ubiquitylation, phosphorylation, and sumoylation.

Histone acetylation and deacetylation are the processes by which the lysine residues within the N-terminal tail protruding from the histone core of the nucleosome are acetylated and deacetylated as part of gene regulation. These reactions are typically catalyzed by enzymes with histone acetyltransferase (HAT) or histone deacetylase (HDAC) activity. Histone acetyltransferases include GNAT family proteins (e.g., Gcn5, Gcn5L, p300/CREB-binding protein associated factor (PCAF), Elp3, HPA2 and HAT1) and MYST family proteins (e.g., Sas3, essential SAS-related acetyltransferase (Esa1), Sas2, Tip60, MOF, MOZ, MORF, and HBO1). Histone deacetylases fall into four classes. Class I includes HDACs 1, 2, 3, and 8. Class II is divided into two subgroups, Class IIA and Class IIB. Class IIA includes HDACs 4, 5, 7, and 9 while Class IIB includes HDACs 6 and 10. Class III contains the Sirtuins and Class IV contains only HDAC11. Classes of HDAC proteins are divided and grouped together based on the comparison to the sequence homologies of Rpd3, Hos1 and Hos2 for Class I HDACs, HDA1 and Hos3 for the Class II HDACs and the sirtuins for Class III HDACs.

The site-specific methylation and demethylation of histone residues are catalyzed by methyltransferases and demethylases, respectively. Histone methylases transfer methyl groups to amino acids (e.g., lysine and arginine) of histone proteins, ultimately effecting transcription of genes. Methylases include SET1, MLL, SMYD3, G9a, GLP, EZH2, and SETDB1. Histone demethylases catalyze the removal of methyl marks from histones, an activity associated with transcriptional regulation and DNA damage repair. Demethylases include, for example, KDM1A, KDM1B, KDM2A, KDM2B, UTX, UTY, Jumonji C (JmJC) domain-containing demethylases, and GSK-J4.

In some embodiments, the at least one effector domains comprise nuclease activity. A nuclease is an agent that induces a break in a nucleic acid sequence, e.g., a single or a double strand break in a double-stranded DNA sequence. Nucleases include those which cut at or near a preselected or specific sequence and those which are not site specific. For example, nucleases include, but are not limited to, zinc finger nucleases (ZFN), homing endonucleases, meganucleases, restriction enzymes, TAL effector nucleases, Argonaute nucleases, CRISPR nucleases, comprising, for example, Cas9, Cpf1, Csm1, CasX or CasY nucleases, micrococcal nuclease, staphylococcal nuclease, DNase 1, T7 endonuclease, or catalytically active fragments thereof.

In some embodiments, the at least one effector domains comprise invertase activity. Invertase activity can be used to alter genome structure by swapping the orientation of a DNA fragment.

In some embodiments, the at least one effector domains comprise recombinase activity. A recombinase is a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3 (also known as TnpR), β-six, CinH, ParA, γδ, Bxb1, ϕC31, TP901, TG1, ϕBT1, R4, ϕRV1, ϕFC1, MR11, A118, U153, and gp29. Examples of tyrosine recombinases include, without limitation, Cre, FLP, R, Lambda, HK101, HK022, and pSAM2. The serine and tyrosine recombinase names stem from the conserved nucleophilic amino acid residue that the recombinase uses to attack the DNA and which becomes covalently linked to the DNA during strand exchange.

In some embodiments, the at least one effector domains comprise resolvase activity. Resolvases are site-specific recombinases that function to excise (as a circle) a segment of DNA contained between two recombination sites (called res) and include, for example, Ruv C resolvase, Holiday junction resolvase Hjc, Tn3 and γδ resolvase.

In some embodiments, the at least one effector domains comprise a peptide or polypeptide sequence responsive to a ligand, such as a hormone receptor ligand binding domain, including, for example, the ligand binding domains of the estrogen receptor, the glucocorticosteroid receptor, and the like. Such effector domains can be used to act as “gene switches,” and be regulated by inducers, such as small molecule or protein ligands, specific for the ligand binding domain.

In some embodiments, the at least one effector domains comprise sequences or domains of polypeptides that mediate direct or indirect protein-protein interactions, including, for example, a leucine zipper domain, a STAT protein N terminal domain, and/or an FK506 binding protein.

In some embodiments, the at least one effector domains comprise DNA editing function (e.g., deaminase, DNA repair activity, DNA damage activity, dismutase activity, alkylation activity, depurination activity, oxidation activity, pyrimidine dimer forming activity, polymerase activity (e.g., reverse transcriptase), ligase activity, helicase activity, photolyase activity or glycosylase activity).

In some embodiments, the activity mediated by the at least one effector domains is a non-biological activity, such as a fluorescence activity (e.g., fluorescent proteins), luminescence activity (e.g., a luminescent protein or enzyme which results in luminescence when interacting with a substrate (e.g., luciferase)), or binding activity, such as those mediated by maltose binding protein (“MBP”), glutathione S transferase (GST), hexahistidine, c-myc, and the FLAG epitope, for facilitating detection, purification, monitoring expression, and/or monitoring cellular and subcellular localization of the polypeptide to which the effector domain is appended. In such embodiments, the systems can also be used as a diagnostic reagent, for example, to detect mutations in gene sequences, to purify restriction fragments from a solution, or to visualize DNA fragments of a gel.

The effector domains described herein are illustrative and merely provide the skilled artisan with examples of effectors that can be used in combination with the systems and methods described herein.

In some embodiments, the at least one effector domain comprises a transcription activator, a transcription repressor, a base editor, an epigenetic modifier, a chromosomal locus imaging agent (e.g., fluorescent protein or protein tag), or a combination thereof.

In some embodiments, the effector domains are fragments of proteins that have been separated from their natural DNA binding domains and engineered to be part of a fusion protein with the components described herein. In some embodiments, the effector domains are proteins which normally bind to other proteins or factors which result in their recruitment to a specific or non-specific nucleic acid.

In some embodiments, Cas7 comprises at least one effector domain. In some embodiments, Cas8 or the Cas8-Cas5 fusion protein comprises at least one effector domain. In some embodiments, Cas12k comprises at least one effector domain. In some embodiments, TniQ comprises at least one effector domain. In some embodiments, TnsC comprises at least one effector domain. In certain embodiments. TnsC is fused at the C-terminus to an effector domain.

In some embodiments, both Cas7 and TnsC comprise at least one effector domain. In some embodiments, the effector domains on Cas7 and TnsC are the same or different type of effector domain. For example, both Cas7 and TnsC may comprise a transcription activator, either the same transcription activator or different transcription activators. Alternatively, Cas7 may comprise a transcription activator, whereas TnsC may comprise a transcription repressor.

b. Nuclear Localization Sequence

In the systems disclosed herein, one or more of the at least one Cas protein and the at least one transposon-associated protein may comprise a nuclear localization signal (NLS). The nuclear localization sequence may be appended to the one or more of the at least one Cas protein and the at least one transposon-associated protein at a N-terminus, a C-terminus, or a combination thereof.

In some embodiments, one or more of the at least one Cas protein and the at least one transposon-associated protein comprises two or more NLSs. The two or more NLSs may be in tandem, separated by a linker, at either end terminus of the protein, or embedded in the protein.

In some embodiments, a NLS is fused to the C-terminus of Cas6. In some embodiments, a NLS is fused to the N-terminus, C-terminus, or both of Cas7. In certain embodiments, Cas7 comprises two NLSs fused in tandem to the N-terminus. In some embodiments, a NLS is fused to the N-terminus or C-terminus of a Cas8-Cas5 fusion protein.

In some embodiments, a NLS is fused to the N-terminus or C-terminus of TniQ. In some embodiments, a NLS is fused to the C-terminus of TnsC. In some embodiments, a NLS is fused to the C-terminus of TnsA. In some embodiments, a NLS is fused to a N-terminus of TnsB.

The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine.

In some embodiments, the NLS is a monopartite sequence. A monopartite NLS comprise a single cluster of positively charged or basic amino acids. In some embodiments, the monopartite NLS comprises a sequence of K-K/R-X-K/R, wherein X can be any amino acid. Exemplary monopartite NLS sequences include those from the SV40 large T-antigen, c-Myc, and TUS-proteins.

In some embodiments, the NLS is a bipartite sequence. Bipartite NLSs comprise two clusters of basic amino acids, separated by a spacer of about 9-12 amino acids. Exemplary bipartite NLSs include the NLS of nucleoplasmin, KR[PAATKKAGQA]KKKK (SEQ ID NO: 2) and the NLS of EGL-13, MSRRRKANPTKLSENAKKLAKEVEN (SEQ ID NO: 3). In some embodiments, the NLS comprises a bipartite SV40 NLS. In certain embodiments, the NLS comprises an amino acid sequence having at least 70% similarity to KRTADGSEFESPKKKRKV(SEQ ID NO: 4). In select embodiments, the NLS consists of an amino acid sequence of KRTADGSEFESPKKKRKV(SEQ ID NO: 4).

The protein components of the disclosed system (e.g., the Cas proteins or the transposon-associated proteins) may further comprise an epitope tag (e.g., 3×FLAG tag, an HA tag, a Myc tag, and the like). In some embodiments, the epitope tag may be adjacent, either upstream or downstream, to a nuclear localization sequence. The epitope tags may be at the N-terminus, a C-terminus, or a combination thereof of the corresponding protein.

c. gRNA

The engineered CRISPR-Cas systems comprise a gRNA complementary to at least a portion of the target nucleic acid sequence, or a nucleic acid encoding the at least one gRNA.

The gRNA may be a crRNA, crRNA/tracrRNA (or single guide RNA, sgRNA). The terms “gRNA,” “guide RNA” and “CRISPR guide sequence” may be used interchangeably throughout and refer to a nucleic acid comprising a sequence that determines the binding specificity of the CRISPR-Cas system. A gRNA hybridizes to (complementary to, partially or completely) a target nucleic acid sequence (e.g., the genome in a host cell). In some embodiments, the at least one gRNA is encoded in a CRISPR RNA (crRNA) array.

The gRNA or portion thereof that hybridizes to the target nucleic acid (a target site) may be between 15-40 nucleotides in length. In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18.19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLoS ONE, 10(3): (2015)); Zhu et al. (PLoS ONE, 9(9) (2014)); Xiao et al. (Bioinformatics. January 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

As described elsewhere herein the protein and gRNA components of the system may be expressed and transcribed from the nucleic acids using any promoter or regulatory sequences known in the art. In some embodiments, the gRNA is transcribed under control of an RNA Polymerase II promoter. In some embodiments, the gRNA is transcribed under control of an RNA Polymerase III promoter.

In some embodiments, the gRNA sequence is at least 50%, 55%, 60%. 65%, 70%, 75%, 80%, 85%, 90%, 95%, 96%. 97%, 98%, 99%, or at least 100% complementary to a target nucleic acid. In some embodiments, the gRNA sequence is at least 50%, 55%. 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The gRNA may be a non-naturally occurring gRNA.

The target nucleic acid may be flanked by a protospacer adjacent motif (PAM). A PAM site is a nucleotide sequence in proximity to a target sequence. For example, PAM may be a DNA sequence immediately following the DNA sequence targeted by the CRISPR-Cas system.

The target sequence may or may not be flanked by a protospacer adjacent motif (PAM) sequence. In certain embodiments, a nucleic acid-guided nuclease can only cleave a target sequence if an appropriate PAM is present, see, for example Doudna et al., Science, 2014, 346(6213): 1258096, incorporated herein by reference. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence) (e.g., for Type I CRISPR/Cas systems). In some embodiments, e.g., Type I systems, the PAM is on the alternate side of the protospacer (the 5′ end). Makarova et al. describes the nomenclature for all the classes, types, and subtypes of CRISPR systems (Nature Reviews Microbiology 13:722-736 (2015)). Guide structures and PAMs are described in by R. Barrangou (Genome Biol. 16:247 (2015)).

Non-limiting examples of the PAM sequences include: CC, CA, AG, GT, TA, AC, CA, GC, CG, GG, CT, TG, GA, AGG, TGG, T-rich PAMs (such as TTIT, TTG, TTC, etc.), NGG, NGA, NAG, NGGNG and NNAGAAW (W=A or T, SEQ ID NO: 6), NNNNGATT (SEQ ID NO: 7), NAAR (R=A or G), NNGRR (R=A or G), NNAGAA (SEQ ID NO: 8) and NAAAAC (SEQ ID NO: 5), where “N” is any nucleotide.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

d. Nucleic Acids

The one or more nucleic acids encoding the engineered CRISPR-Cas system may be any nucleic acid including DNA, RNA, or combinations thereof. In some embodiments, the one or more nucleic acids comprise one or more messenger RNAs, one or more vectors, or any combination thereof.

The at least one Cas protein, the at least one transposon-associated protein, and the at least one gRNA may be on the same or different nucleic acids (e.g., vector(s)). In some embodiments, wherein the at least one transposon-associated protein is encoded on a same or different nucleic acid as the at least one Cas protein and the gRNA. In some embodiments, the at least one Cas protein and the at least one transposon-associated protein are encoded by a single nucleic acid. In some embodiments, each of the at least one Cas protein, the at least one transposon-associated protein, and the gRNA are encoded by a single nucleic acid.

In some embodiments, the at least one gRNA is encoded by a nucleic acid different from the nucleic acid(s) encoding the at least one Cas protein and at least one transposon-associated protein.

In some embodiments, the at least one gRNA is encoded by a nucleic acid also encoding the at least one Cas protein, the at least one transposon-associated protein, or both. In select embodiments, a single nucleic acid encodes the gRNA and at least one Cas protein. For example, in certain embodiments, a single nucleic acid encodes the gRNA and Cas6. In alternative embodiments, a single nucleic acid encodes the gRNA and Cas7.

The gRNA may be encoded anywhere in the nucleic acid encoding the at least one Cas protein. In some embodiments, the gRNA is encoded in the 3′ UTR of the Cas protein.

The one or more nucleic acids encoding the protein components may further comprise, in the case of RNA, or encode, as in the case of DNA, a sequence capable of forming a triple helix adjacent to the sequence encoding the protein component. In some embodiments, the sequence capable of forming a triple helix is downstream of the sequence encoding the at least one Cas protein and/or the sequence encoding the at least one transposon-associated protein. In some embodiments, the sequence capable of forming a triple helix is in a 3′ untranslated region of the sequence encoding the at least one Cas protein or the sequence encoding the at least one transposon-associated protein.

A tiple helix is formed after the binding of a third strand to the major groove of a duplex nucleic acid through Hoogsteen base pairing (e.g., hydrogen bonds) while maintaining the duplex structure of two strands making the major groove. Pyrimidine-rich and purine-rich sequences (e.g., two pyrimidine tracts and one purine tract or vice versa) can form stable triplex structures as a consequence of the formation of triplets (e.g., A-U-A and C-G-C).

In some embodiments, the triple helix forming sequence comprises two uracil-rich tracts and an adenosine-rich tract, each separated by linker or loop regions. As used herein, the term “A-rich tract” refers to a strand of consecutive nucleosides in which at least 80% of the consecutive nucleosides are adenosine. Similarly, the term “U-rich motif” refers to a strand of consecutive nucleosides in which at least 80% of the consecutive nucleosides are uridine.

In some embodiments, the triple helix sequence is derived from the 3′ terminal triple helix sequences of triple helix terminators from a long non-coding RNAs (lncRNAs), e.g., metastasis-associated lung adenocarcinoma transcript 1 (MALAT1).

One or more of the at least one Cas protein and the at least one transposon-associated protein comprise a sequence of an internal ribosome entry site (IRES) or a ribosome skipping peptide. This is particularly advantageous when a single nucleic acid or vector is used to express multiple components of the system.

The ribosome skipping peptide may comprise a 2A family peptide. 2A peptides are short (˜18-25 aa) peptides derived from viruses. There are four commonly used 2A peptides, P2A, T2A, E2A and F2A, that are derived from four different viruses. Any known 2A peptide sequence is suitable for use in the disclosed system.

In certain embodiments, engineering the system for use in eukaryotic cells may involve codon-optimization. It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., 65%, 70%, 75%, 80%, 85%, 90%, 95%, or 98%) of the codons encoded therein are mammalian preferred codons. Furthermore, in some embodiments, engineering the CRISPR-Cas system involves incorporating elements of the native CRISPR array into the disclosed system.

The present disclosure also provides for DNA segments encoding the proteins and nucleic acids disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more or all of the components of the present system. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. For example, this may allow for DNA integration by transforming bacteria of interest, but then being left with engineered strains that have no memory of the plasmids or vectors used for the integration.

A variety of viral constructs may be used to deliver the present system (such as one or more Cas proteins and/or transposon-associated proteins, gRNA(s), etc.) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

In one embodiment, a DNA segment encoding the present protein(s) is contained in a plasmid vector that allows expression of the protein(s) and subsequent isolation and purification of the protein produced by the recombinant vector. Accordingly, the proteins disclosed herein can be purified following expression, obtained by chemical synthesis, or obtained by recombinant methods.

To construct cells that express the present system, expression vectors for stable or transient expression of the present system may be constructed via conventional methods as described herein and introduced into host cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see. e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression of a RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter. GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′-and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

The present system (e.g., proteins, polynucleotides encoding these proteins, and compositions comprising the proteins and/or polynucleotides described herein) may be delivered by any suitable means. In certain embodiments, the system is delivered in vivo. In other embodiments, the system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83), incorporated herein by reference.

Exemplary vectors encoding the systems described herein are provided in SEQ ID NO: 14-55.

### Methods

Disclosed herein are methods for utilizing the disclosed systems in cell. In some embodiments, the methods are directed to recruiting one or more effectors domains to a target nucleic acid in a cell. In some embodiments, the methods are directed to modulating expression of a target gene in a cell. The methods may comprise introducing the disclosed systems into a cell. The descriptions and embodiments provided above for the engineered CRISPR-Cas system, the gRNA, and the effector domains are applicable to the methods described herein.

As described above the system may be introduced into eukaryotic or prokaryotic cells by methods known in the art. In some embodiments, the cell is a eukaryotic cell. In some embodiments, the cell is a mammalian cell (e.g., human cell). In some embodiments, the cell is prokaryotic cell.

In some embodiments, the target nucleic acid is a nucleic acid endogenous to a target cell. In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

In some embodiments, the target nucleic acid comprises a promoter region of a gene of interest. In some embodiments, the target nucleic acid comprises an upstream activator sequence. In some embodiments, the gene of interest is located on a chromosome in a cell.

Polynucleotides containing the target nucleic acid sequence may include, but is not limited to, purified chromosomal DNA, total cDNA, cDNA fractionated according to tissue or expression state (e.g., after heat shock or after cytokine treatment other treatment) or expression time (after any such treatment) or developmental stage, plasmid, cosmid, BAC, YAC, phage library, etc. Polynucleotides containing the target site may include DNA from organisms such as Homo sapiens, Mus domesticus, Mus spretus, Canis domesticus, Bos, Caenorhabditis elegans, Plasmodium falciparum, Plasmodium vivax, Onchocerca volvulus, Brugia malayi, Dirofilaria immitis, Leishmania, Zea maize, Arabidopsis thaliana, Glycine max, Drosophila melanogaster, Saccharomyces cerevisiae, Schizosaccharomyces pombe, Neurospora, Escherichia coli, Salmonella typhimurium, Bacillus subtilis, Neisseria gonorrhoeae, Staphylococcus aureus, Streplococcus pneumonia, Mycobacterium tuberculosis, Aquifex, Thermus aquaticus, Pyrococcus furiosus, Thermus littoralis, Methanobacterium thermoaulotrophicum, Sulfolobus caldoaceticus, and others.

The method may comprise administering to the subject, in vivo, or by transplantation of ex vivo treated cells, an effective amount of the described system. In some embodiments, the vector(s) is delivered to the tissue of interest by, for example, an intramuscular, intravenous, transdermal, intranasal, oral, mucosal, or other delivery methods.

The components of the present system or ex vivo treated cells may be administered with a pharmaceutically acceptable carrier or excipient as a pharmaceutical composition. In some embodiments, the components of the present system may be mixed, individually or in any combination, with a pharmaceutically acceptable carrier to form pharmaceutical compositions, which are also within the scope of the present disclosure.

In some embodiments, an effective amount of the components of the present system or compositions as described herein can be administered. Within the context of the present disclosure, the term “effective amount” refers to that quantity of the components of the system such that recruitment of one or more effector domains and, if desired, modulation of expression of a target gene is achieve.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean eliminate or reduce a patient's tumor burden, or prevent, delay, or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

### Kits

Also within the scope of the present disclosure are kits that include the components of the present system.

The kit may include instructions for use in any of the methods described herein. The instructions can comprise a description of administration of the present system or composition to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The kits provided herein are in suitable packaging. Suitable packaging includes, but is not limited to, vials, bottles, jars, flexible packaging, and the like. A kit may have a sterile access port (for example, the container may be an intravenous solution bag or a vial having a stopper pierceable by a hypodermic injection needle). The container may also have a sterile access port.

The packaging may be unit doses, bulk packages (e.g., multi-dose packages) or subunit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Kits optionally may provide additional components such as buffers and interpretive information. Normally, the kit comprises a container and a label or package insert(s) on or associated with the container. In some embodiment, the disclosure provides articles of manufacture comprising contents of the kits described above. Optional components of the kit include one or more of the following: buffer constituents, control plasmid, sequencing primers, cells.

The kit may further comprise a device for holding or administering the present system or composition. The device may include an infusion device, an intravenous solution bag, a hypodermic needle, a vial, and/or a syringe.

### EXAMPLES

The following are examples of the present invention and are not to be construed as limiting.

Experimental results presented herein and described in accompanying figures employed a large set of variable gRNA and protein expression vectors. Results presented in bar graphs and elsewhere are accompanied by an experimental numeric, which is linked with information provided in Table 1. This table provides a key describing the vectors (aka plasmids) that were used, for the same experimental numeric ID. Plasmid descriptions are provided in Table 2.

### Example 1

### Cascade-Based Transcriptional Repression Activity in Human Cells

The TniQ-Cascade complex encoded by Tn6677 is an RNA-guided DNA binding complex, comprising crRNA (aka guide RNA, in one copy), Cas8 (one copy), Cas7 (six copies), Cas6 (one copy) and TniQ (two copies) (Klompe et al., Nature 571, 219-225 (2019); Halpin-Healy et al., Nature 577, 271-274 (2020)). Based on the ability to purify Cascade without the TniQ subunit, and previous studies of Type I-F Cascade (aka Csy complex) from canonical I-F1 CRISPR-Cas systems, TniQ is not essential for formation of the complex and RNA-guided DNA targeting and binding.

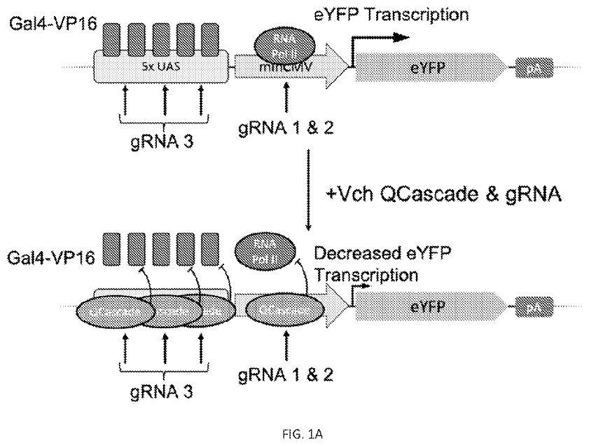

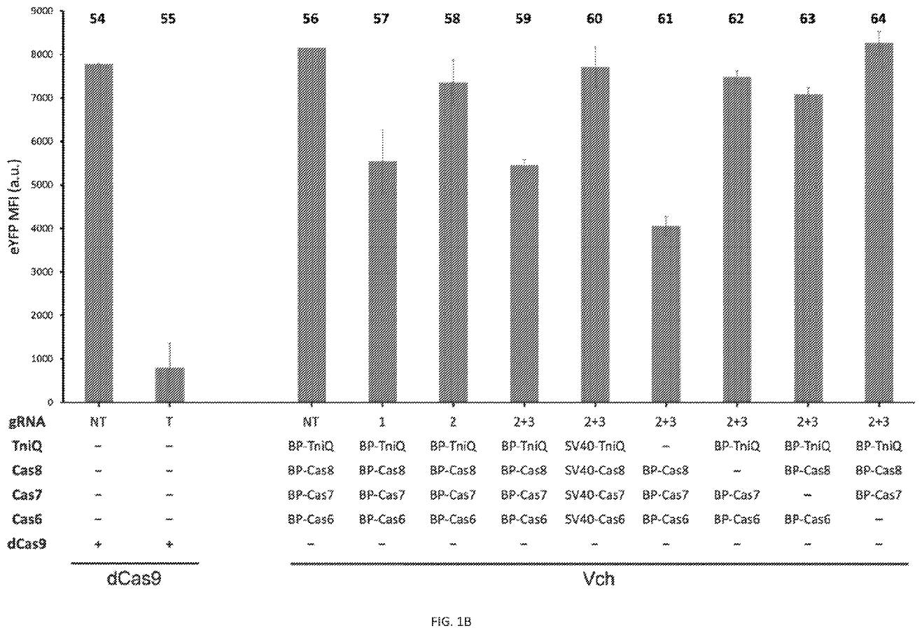

A fluorescence-based transcriptional repression assay was developed to monitor DNA binding activity of V. cholerae TniQ-Cascade in human cells. This assay was based on the ability of programmable DNA binding proteins to either block RNA Pol II recruitment to a minimal CMV promoter upstream of an eYFP reporter gene, or to block binding of a Gal4-VP16 synthetic transcriptional activator to an upstream activation sequence (UAS) cloned upstream of the minimal CMV promoter on the eYFP reporter plasmid (FIGS. 1A & 1B). In control experiments, S. pyogenes dCas9 programmed by an appropriate sgRNA is able to repress eYFP expression by 90 percent upon HEK293T co-transfection with the eYFP reporter plasmid, a Gal4-VP16 expression plasmid, and the Cas9-sgRNA expression plasmid (Yeo et al., Nature Methods 15, 611-616 (2018); FIG. 1B).

VchINTEGRATE gRNAs were designed to target either the minimal CMV promoter directly (gRNA-1 and gRNA-2), or the UAS upstream of the eYFP reporter construct (gRNA-3; FIGS. 1A & 1B). Notably, because of the tandem array nature of the UAS, the UAS-specific gRNA-3 has three identical target sites within the full-length UAS element. When HEK293T cells were transfected with the reporter plasmid, a VchINTEGRATE gRNA expression plasmid driven by a human U6 promoter, and expression plasmids encoding TniQ, Cas8, Cas7, and Cas6 with appropriately designed NLS tags, clear evidence of transcriptional repression was observed (FIG. 1B). This repression activity was absent when a non-targeting gRNA was used. Controls in which plasmids encoding TniQ, Cas8, Cas7, or Cas6 were omitted were completed and repression activity was absent when Cas8, Cas7, or Cas6 was omitted; the absence of TniQ did not completely ablate repression activity (FIG. 1B).

### Example 2

### Cascade and TniQ-Cascade-Based Transcriptional Activation Activity in Human Cells

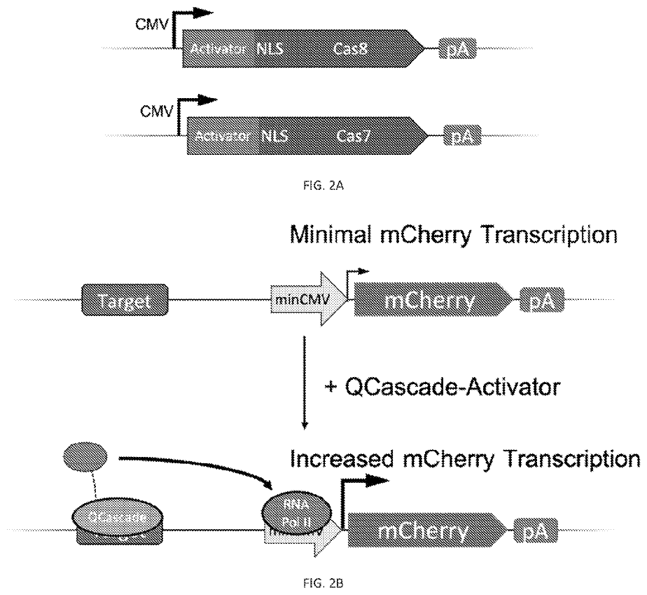

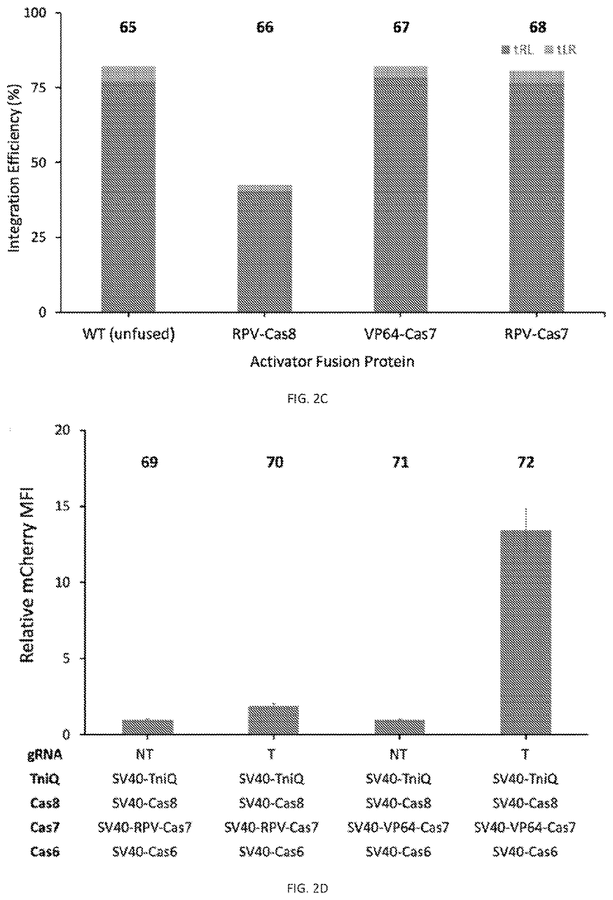

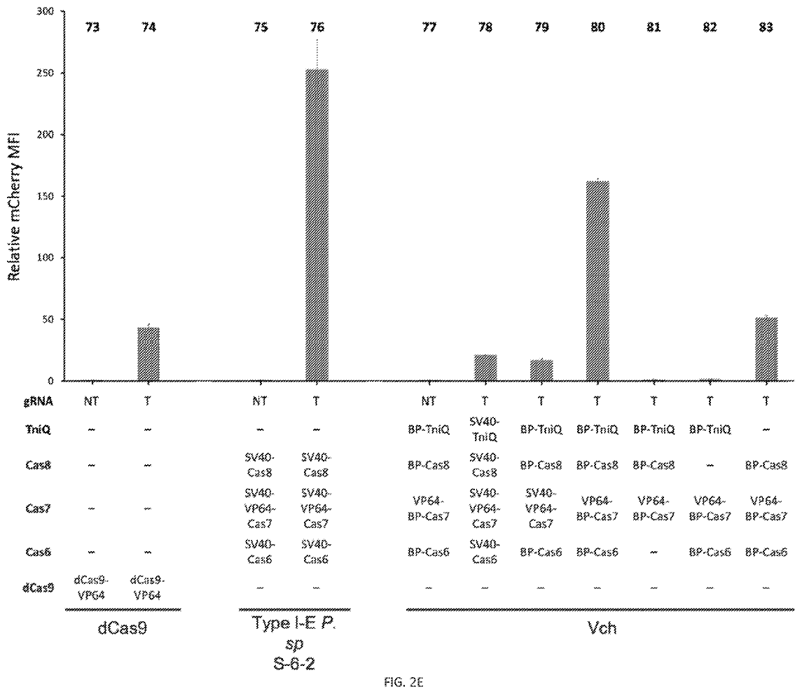

QCascade-based activators were engineered and tested for transcriptional activation activity in a fluorescence-based reporter plasmid assay (FIG. 2B). An mCherry reporter gene was placed downstream of a minimal CMV promoter, such that basal mCherry expression, and thus cellular mCherry fluorescence, was minimal. If a synthetic transcription factor successfully bound to a region upstream of the minimal CMV promoter, this would drive recruitment of RNA Polymerase II to the minimal promoter, thereby resulting in a large increase in mCherry expression. This reporter approach was validated using either a nuclease-dead Cas9 from S. pyogenes fused to the VP64 transcriptional activator (Mali et al., Nature Biotechnol 31, 833-838 (2013)), or a Type I-E Cascade complex derived from Pseudomonas sp. S-6-2 (as described by Cameron et al., Nat Biotechnol 37, 1471-1477 (2019)), with VP64 fused to the C-terminus of the Cas7 subunit. These experiments showed that mCherry transcriptional activation could be robustly observed using appropriately designed gRNAs, and that this activity was absent with non-targeting gRNA controls (FIG. 2E).

A panel of transcriptional activators was generated for V. cholerae TniQ-Cascade (also referred to simply as QCascade). Activator domains may be appended to Cas8, Cas7, Cas6, TniQ, or a combination thereof (see for example FIG. 2A). Fusions of VP64 or RPV (a reversed order of the previously described VPR activator, comprising VP64-p65-Rta domains) to the N-terminus of Cas7, together with an NLS tag upstream of the activator fusion, were generated. A similar fusion protein was generated on a bacterial TniQ-Cascade expression plasmid and tested RNA-guided DNA integration in E. coli. Efficient DNA integration was observed with or without the presence of a mammalian activation domain fused to Cas7 (FIG. 2C) demonstrating that these fusions did not compromise the ability of QCascade to bind target DNA and recruit downstream transposase (e.g., TnsC) components.

Cas7-activator fusions were generated in the context of a mammalian expression vector, and HEK293T cells were co-transfected with the reporter plasmid, a gRNA expression plasmid targeting the region upstream of mCherry and minimal CMV promoter (FIG. 2B), and protein expression plasmids encoding the VP64-Cas7 fusion, Cas8, Cas6, and TniQ. After evaluating mCherry expression levels by flow cytometry, VchINTEGRATE-derived QCascade exhibited potent transcriptional activation activity, as assayed by a large, 162-fold increase in cellular mCherry fluorescence levels (FIGS. 2D & 2E). Certain gRNAs were more potent in transcriptional activation than others, suggesting sequence- and/or position-dependent effects in the robustness of transcriptional activation (FIG. 2E and Table 1). The RNA-guided transcriptional activation activity mediated by VchINTEGRATE QCascade was lost when we used a non-targeting gRNA. Additional controls, in which plasmids encoding TniQ, Cas8, or Cas6 were omitted, determined that transcriptional activation activity was completely eliminated when Cas8 or Cas6 were omitted. The absence of TniQ reduced, but did not eliminate, transcriptional activation (FIG. 2E).

The protein expression plasmids were modified so that the SV40 NLS tag was replaced with a bipartite (BP) NLS tag. When similar transcriptional activation experiments as described above were performed the level of activation was substantially higher with BP NLS tags instead of monopartite (SV40) NLS tags (FIG. 2E; compare bars numbered 78-80).

### Example 3

### TniQ-Cascade and TnsC-Based Transcriptional Activation Activity in Human Cells

Previous studies of the E. coli Tn7 transposon have shown that TnsC is a AAA+ ATPase that has specific interactions with TnsD, a TniQ-family protein that binds the conserved attachment site in the glmS gene. TnsC is a regulator protein that engages TnsA and TnsB, and indeed, it has specific interactions with both proteins and is thought to bridge interactions between the targeting module (TnsD in Tn7) and the excision/integration module (TnsA and TnsB). VchTnsC derived from Tn6677 mediates RNA-guided DNA integration activity.

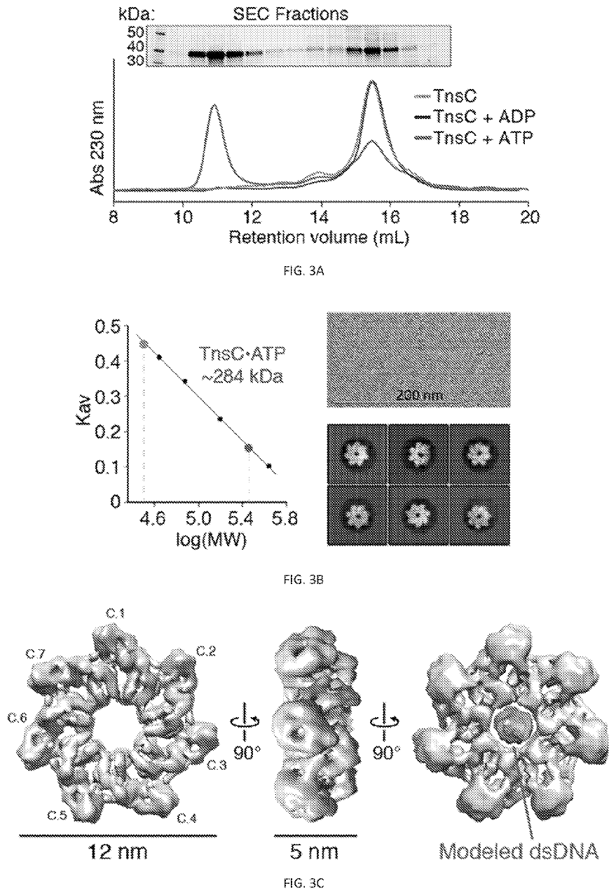

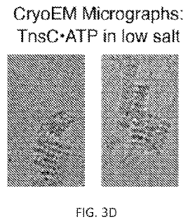

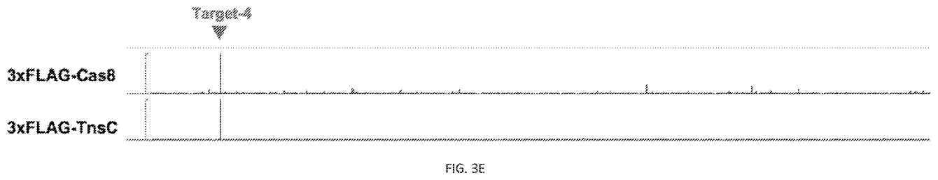

VchTnsC can be purified from bacteria in a monomeric state, which remains soluble only under high ionic strength conditions (e.g., 1 M monovalent salt, such as sodium chloride). In the presence of ATP or ATP analogs, however, VchTnsC forms a stable heptamer species that remains soluble under physiological buffer and salt conditions, and forms a ring-like architecture that is expected to nucleate around a nucleic acid substrate (FIGS. 3A-3C). In addition, TnsC can form larger stacked rings under certain conditions, as visualized by negative stain electron microscopy (FIG. 3D). In E. coli experiments, VchTnsC is specifically recruited to genomic target sites bound by TniQ-Cascade, with excellent genome-wide fidelity as assayed by chromatin immunoprecipitation experiments followed by next-generation sequencing (ChIP-seq; FIG. 3E).

Synthetic transcriptional activators were constructed using the TniQ-Cascade and TnsC components derived from Tn6677 to specifically test if using TniQ-Cascade and TnsC would allow downstream applications with more potency and dynamic range than would be possible with a single-copy effector protein like dCas9.

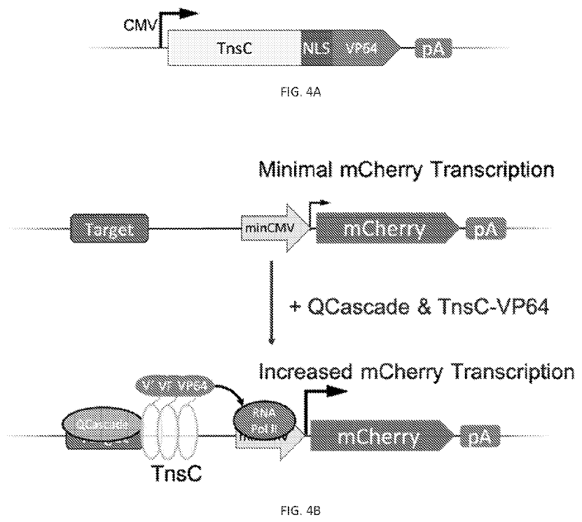

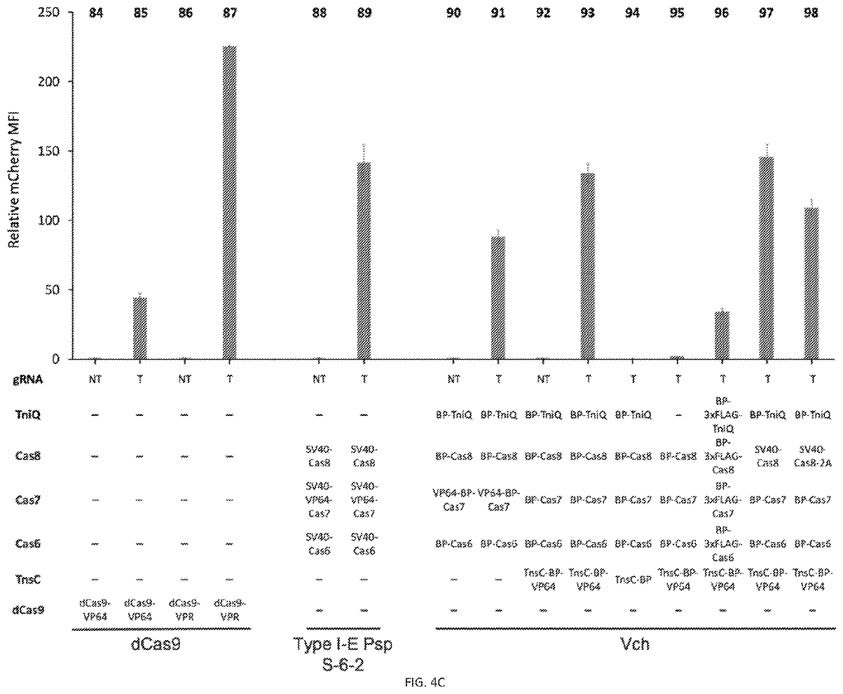

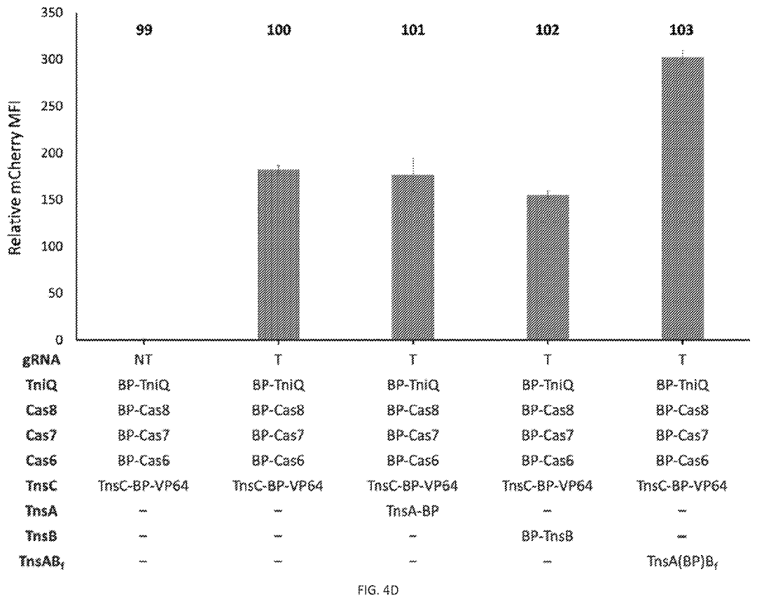

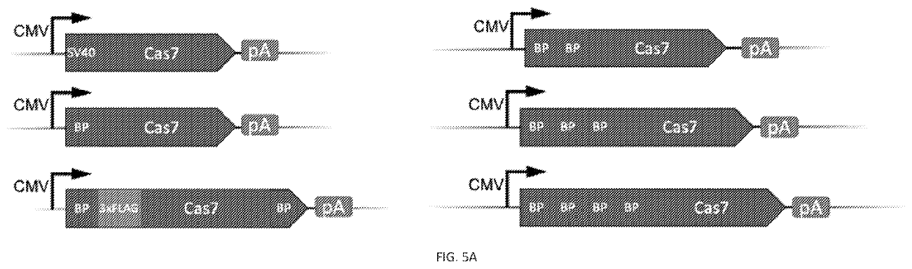

V. cholerae TnsC was fused to a VP64 transcriptional activation domain (FIG. 4A) and HEK293T cells were co-transfected with the mCherry reporter plasmid described above, a gRNA expression plasmid, and protein expression plasmids encoding Cas8, Cas7, Cas6, TniQ, and TnsC-VP64 activators. In these experiments, the protein components may have no NLS tags, SV40 NLS tags, bipartite (BP) NLS tags, or a combination thereof. Targeting of the mCherry reporter plasmid by TniQ-Cascade in a gRNA-dependent manner would lead to stable binding, thereby leading to subsequent recruitment of many copies of the intrinsically oligomeric TnsC polypeptide (FIGS. 4A and 4B). Because TnsC is fused to the VP64 activator domain, the recruitment of multiple copies of TnsC to a localized site elicited a potent and robust activation of gene expression through multivalency.

A much greater activation of mCherry expression was observed when compared with VP64 fused to the Cas7 subunit of QCascade, dCas9, or a Type I-E Cascade complex derived from Pseudomonas sp. S-6-2 with VP64 fused to the C-terminus of Cas7 subunit (FIG. 4C). Activation was dependent on the gRNA targeting the matching sequence upstream of the minimal CMV promoter, and was lost when the TniQ expression plasmid was omitted or a non-targeting gRNA was used instead (FIG. 4C). When similar experiments were performed including TnsA, TnsB, or TnsAB_{f} expression plasmids, the level of transcriptional activation was increased by approximately 2× when TnsAB_{f} was included, suggesting that the presence of TnsAB_{f} stabilizes and/or stimulates TnsC recruitment to the target site marked by QCascade (FIG. 4D). Different combinations of NLS, epitope (3×FLAG), or 2A motifs led to varied levels of transcriptional activation (FIG. 4C).

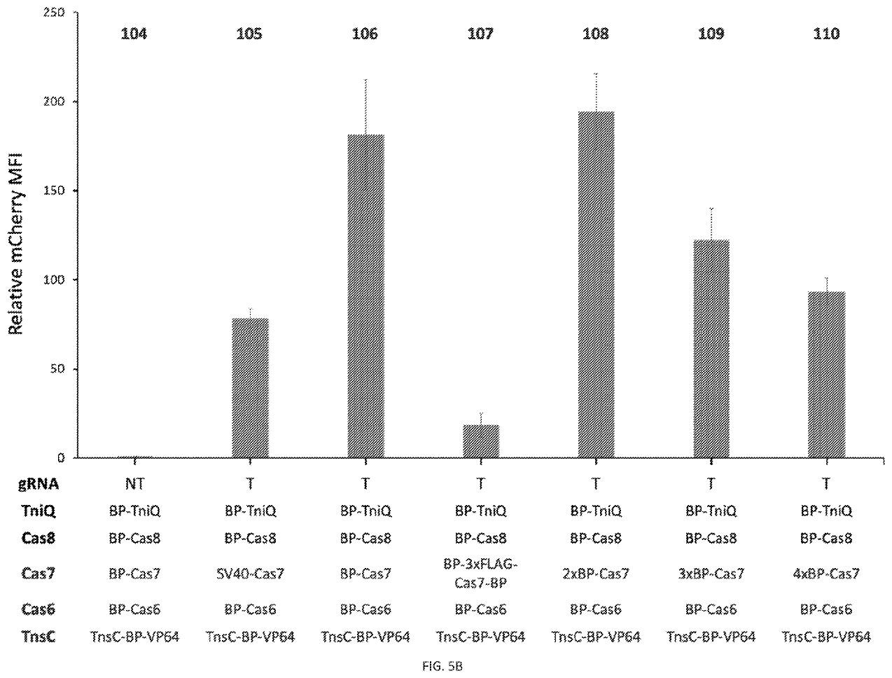

Data suggested that substitution of SV40 NLS tags with BP NLS tags for Cas7 can affect QCascade complex formation and targeting (FIG. 2E). Additional permutations of Cas7, including multiple tandem BP NLS tags, and/or combinations of NLS tags and 3×FLAG epitope tags were tested (FIG. 5A). Using the same TnsC-VP64 activation assay described above, QCascade and TnsC activity was substantially increased when only Cas7 was switched from an SV40 NLS to a BP NLS (compare bars 105 and 106), and that a 2×BP-NLS tag slightly increased TnsC transcriptional activation. In contrast, the addition of more BP-NLS tags led to a decrease of transcriptional activation, suggesting a negative effect of Cas7 expression and/or activity (FIG. 5B).

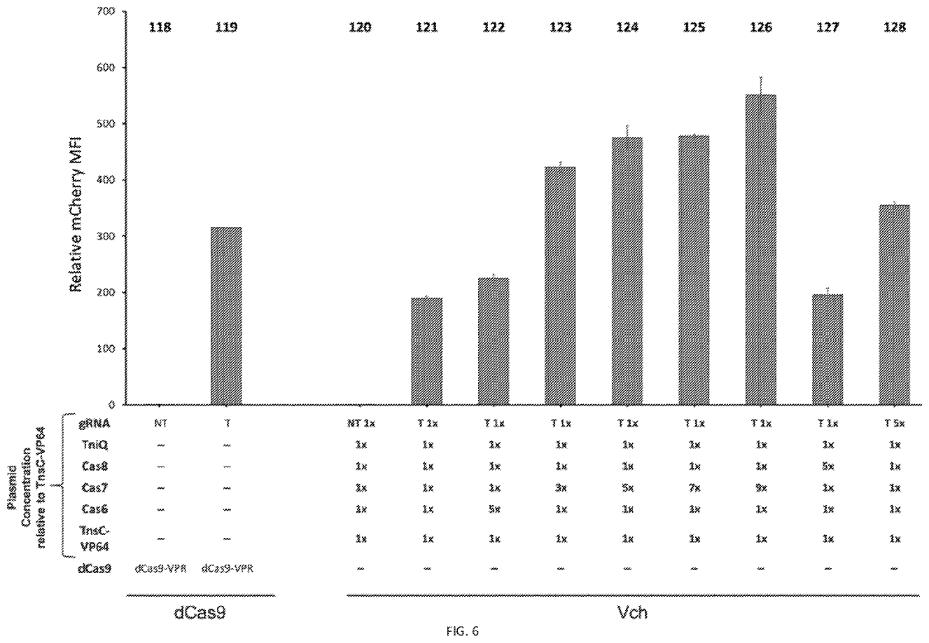

The relative concentration of a Cas7 expression plasmid was sequentially increased compared to all other components, and resulted in a dose-dependent increase in mCherry activation via the previously described TnsC-based transcriptional activation assay (FIG. 6). Additionally, increasing the relative concentration of other subunits resulted in limited increases in mCherry activation, and in some cases a reduction in mCherry activation (FIG. 6). In some cases, stronger mCherry activation was generated than seen with dCas9-VPR, underscoring the potency of TnsC as a transcriptional activator because of its multivalency upon recruitment to DNA-bound QCascade.

The ability of TnsC-VP64 to transcriptionally induce endogenous gene expression was profiled in HEK293T cells (FIG. 10A). Protein expression plasmids of TnsC-VP64, Cas8, Cas7, Cas6, and TniQ were transfected alongside 3 or 4 crRNA expression plasmids targeting endogenous genes TTN, MIAT, ASCL1, and ACTC1 (FIG. 10D). The relative gene expression of targeted loci was measured via quantitative reverse transcription PCR (RT-qPCR). For the TTN locus, controls conditions included in the genomic activation experiments included transfection of plasmids encoding all necessary protein components and a non-targeting crRNA, all necessary protein components and a TnsC lacking a VP64 domain, and all necessary protein components except for TniQ. Relative to the non-targeting conditions, experimental samples that received 4 separate crRNA expression plasmids displayed a 278-fold increase in TTN expression (FIG. 10B). Conditions that lacked either TniQ or a VP64 tag fused to TnsC did not exhibit robust transcriptional induction. TTN activation by TnsC-VP64 greatly exceeded activation at the same locus by dCas9-VP64 and demonstrated similar activation compared to dCas9-VPR, highlighting the potency of TnsC as a result of its multivalent recruitment to genomic target sites in human cells. Similarly, transcriptional activation was observed for the other 3 genomic loci profiled, at levels ranging from 35-fold for ACTC1 to 1290-fold for ASCL1 (FIG. 10E).

TnsC-VP64 genomic transcriptional induction was profiled as a function of crRNA expression plasmid delivery. HEK293T cells were transfected with plasmids expressing all necessary protein components and either 4 crRNA expression plasmids targeting TN, or 1 plasmid expressing an unprocessed CRISPR array containing 4 spacer sequences targeting the same region of TTN as the individual crRNA plasmids (FIG. 10C). While both the individual crRNA plasmids and the CRISPR array plasmid, hereinafter referred to as the “multiplexed crRNA” plasmid, displayed robust TTN expression induction, the multiplexed crRNA plasmid demonstrated higher levels of TTN expression. This work highlights the advantageous mechanism through which Cas6 can process multiple crRNAs from one CRISPR array, facilitating both targeting multiple sequences within a gene of interest, or using one multiplexed crRNA plasmid to target multiple different genes within one cell.

### Example 4

### TniQ-Cascade and TnsC-Based Genome Engineering, Epigenome Engineering, and Chromosome Imaging

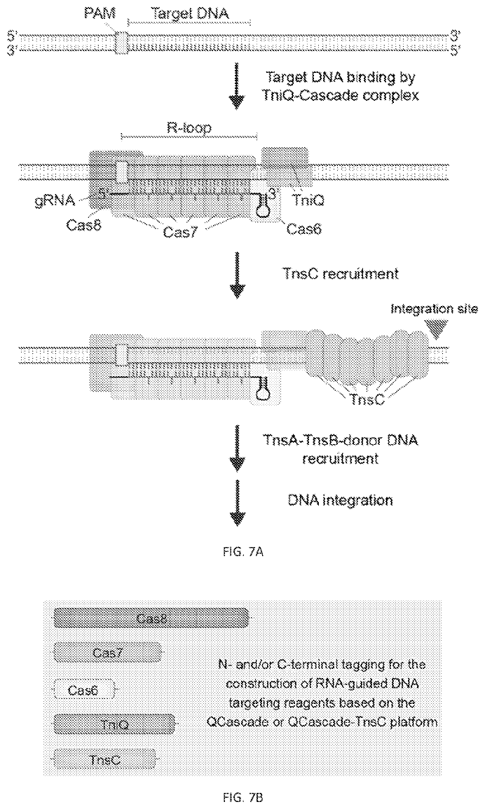

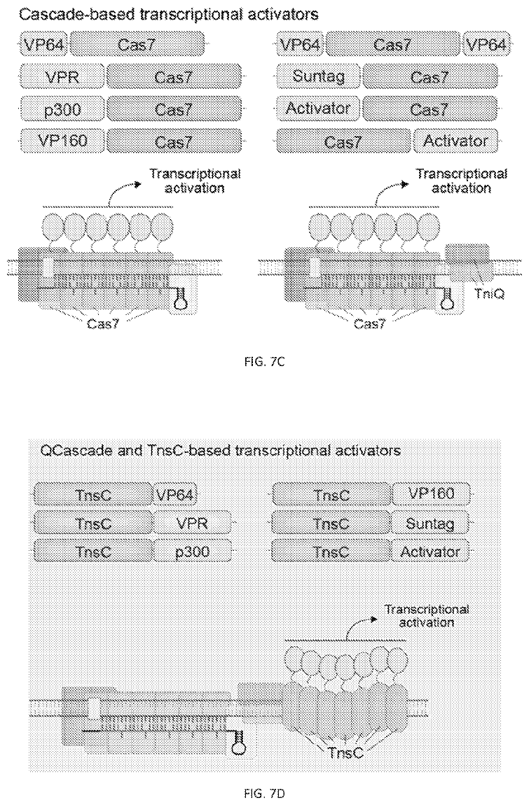

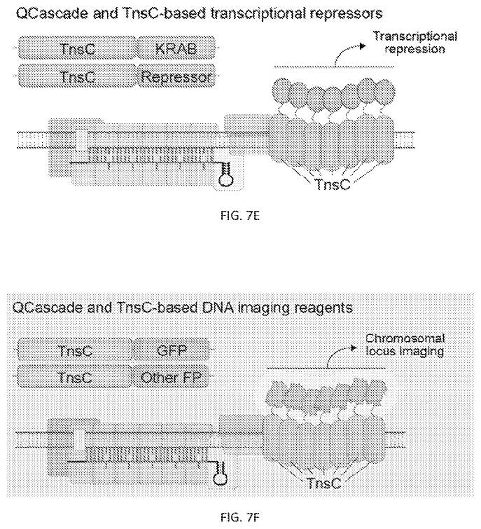

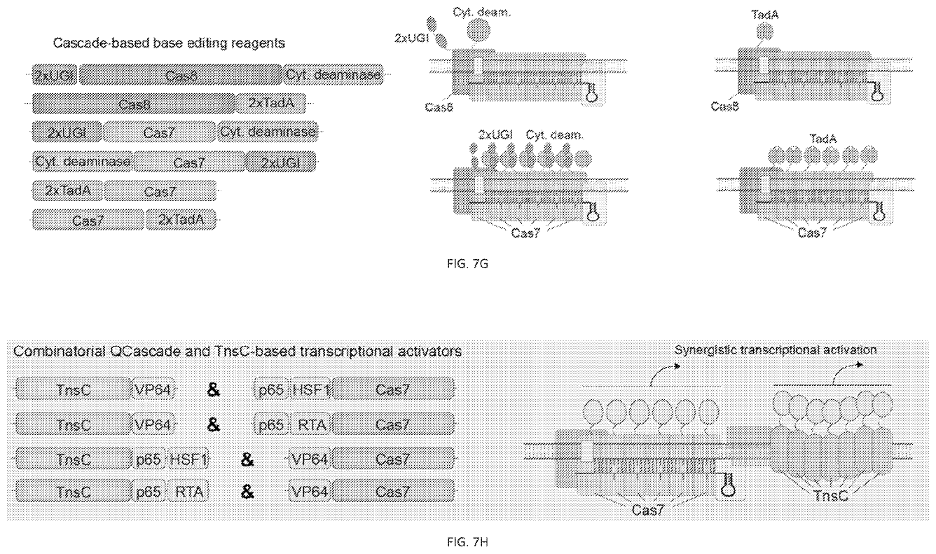

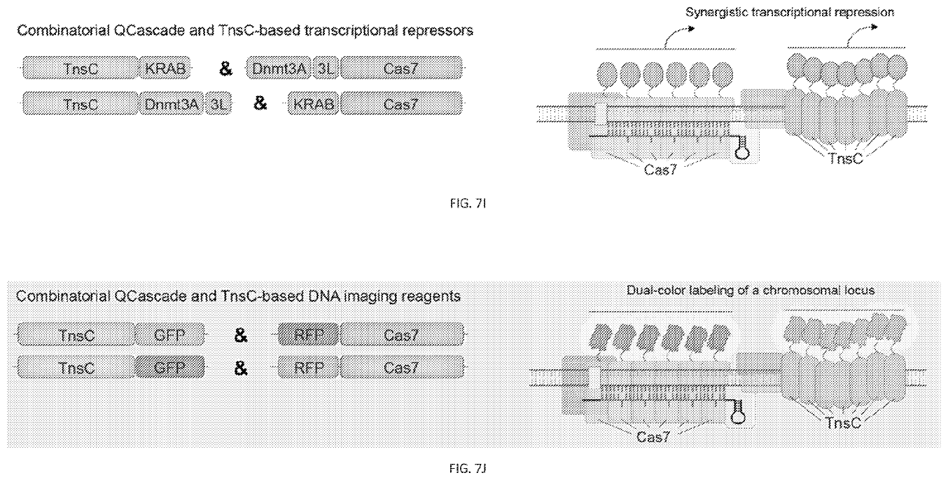

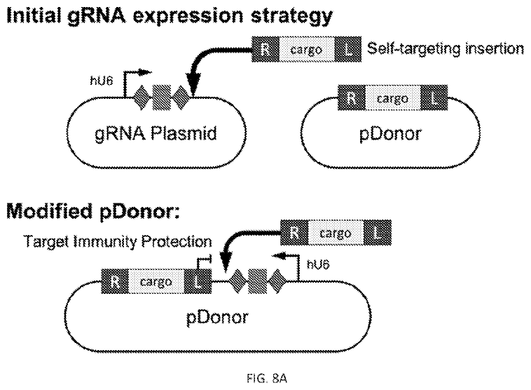

RNA-guided DNA targeting by QCascade, and the subsequent recruitment of TnsC, result in high-copy localization of both Cas7 and TnsC (FIG. 7A). In the case of Cas7, a typical gRNA for I-F CRISPR-Cas systems results in 6 copies of Cas7 being present, though the gRNA may also be extended for the formation of even larger R-loops ((Klompe et al., Nature 571, 219-225 (2019); Halpin-Healy, T. S. et al., Nature 577. 271-274 (2020); Songailiene, I. et al. Cell Rep 28, 3157-3166.e4 (2019)). The exact number of TnsC copies recruited during the subsequent step is not known, though it is likely to be at least 7. Thus, based on the results described above, by tethering effector domains to either the N- or C-terminus of one or more protein components of QCascade and TnsC, a variety of novel genome engineering tools are constructed (FIG. 7B).

A series of embodiments are presented in FIG. 7 that describe eukaryotic transcriptional activation tools (CRISPRa), transcriptional repression tools (CRISPRi), base editing tools (CBE and ABE), and chromosomal locus imaging tools based on the systems and methods described herein. Also described is the combinatorial addition of multiple distinct effector domains on both a component of Cascade (e.g., Cas7) and on TnsC, such that synergistic activity of both fusion proteins, present in high copies, leads to potent and durable perturbations in a highly-specific, targeted manner using just one gRNA.

The same approaches may be applied to homologous CRISPR-transposon systems, which may derive from either Type I-F (e.g., homologous to VchINTEGRATE), Type I-B (e.g., homologous to AvCAST; Saito, M. et al. Cell 184, 2441-2453.e18 (2021)), or Type V-K (e.g., homologous ShoINTEGRATE; Vo, P. L. H. et al. Nat Biotechnol 359, eaan4672 (2020)). In the case of homologous Type I-F and Type I-B systems, the same fusion strategies as described in FIG. 7 may be applied; the only substantive differences between these systems, in terms of the protein components necessary for RNA-guided DNA targeting and integration, is that Type I-F encodes a natural Cas8-Cas5 fusion (referred to herein simply as Cas8), whereas the Type I-B system encodes separate Cas8 and Cas5 polypeptides. Additionally, Type I-B systems encode TnsA-TnsB fusion polypeptides. In embodiments in which components from Type V-K systems are used, effector fusions may still be generated with TnsC, which is similarly present and is recruited to genomic target sites in multiple copies. Alternatively, fusion constructs may be generated with Cas12k, or combinatorial effector fusions are made to Cas12k and TnsC, or TniQ and TnsC, or Cas12k and TniQ, or Cas12k and TniQ and TnsC.

Cascade-based transcriptional activators may be constructed by fusing VP64 to the N-terminus of Cas7, together with appropriate nuclear localization signals (NLS). In addition to the VP64-TnsC fusions described (FIG. 2), a range of other activation domains may be utilized (FIG. 7C). Cas7-based transcriptional activators may be used in the context of the Cascade complex alone (e.g., lacking TniQ), or in the context of the QCascade complex. The multi-valent recruitment of Cas7 to the target site, in 6 or more copies, depending on the length of the gRNA, leads to potent transcriptional activation in response to target with just a single gRNA.

RNA-guided transcriptional activators may also be generated by fusing transcriptional activation domains to the C-terminus of TnsC. In addition to the extensive data provided for TnsC-VP64 activators (FIGS. 4-6), TnsC may be fused to a wide range of alternative activation or epigenome modification domains (FIG. 7D). An NLS is included (but not shown in the figure), and may be encoded at the C-terminus, or in between TnsC and the effector domain, or internally. TnsC is recruited to genomic target sites by the QCascade complex, and is recruited in multiple copies, such that the high valency leads to potent activity of the fused effector domains (FIG. 7D).

TnsC may alternatively be fused to transcriptional repression domains, such as KRAB domains or other repressive domains (FIG. 7E). An NLS is included (but not shown in the figure), and may be encoded at the C-terminus, or in between TnsC and the effector domain, or internally. TnsC is recruited to genomic target sites by the QCascade complex, and is recruited in multiple copies, such that the high valency leads to potent activity (FIG. 7E).

TnsC may also be fused to fluorescent proteins (FPs), such as GFP, for chromosomal labeling (FIG. 7F). An NLS is included (but not shown in the figure), and may be encoded at the C-terminus, or in between TnsC and the FP domain, or internally. TnsC is recruited to genomic target sites by the QCascade complex, and is recruited in multiple copies, such that the high valency leads to high signal-to-noise localization of multiple chromophores at the same target site, in response to targeting by just one gRNA (FIG. 7F).

Cas8 or Cas7 may be fused to base editing reagents (FIG. 7G), as described (Anzalone et al., Nat Biotechnol 38, 824-844 (2020) and references therein). Various fusions allow variable windows of base editing across the 32-nucleotide R-loop, or smaller/larger R-loops in the case of modified gRNAs containing distinct lengths of spacer. In the case of cytosine base editors (CBEs), the target Cascade component is fused to both the deaminase domain as well as uracil glycosylase inhibitor domains. In the case of adenine base editors (ABEs), the target Cascade component is fused to two tandem TadA domains, one of which is evolved to deaminate deoxyadenosine. In the case of Cas7-CBE and Cas7-ABE base editors, a large window of the R-loop is subjected to potent deamination, given the multiple copies of Cas7 throughout the Cascade complex. Cascade base editors may also be combined with Cas9 nickase enzymes, in order to nick one strand of DNA and thereby improve purity of the final product.

QCascade and TnsC also offer opportunities for combinatorial fusion of multiple effector domains to distinct protein components, to allow synergistic responses (FIGS. 7H-7J). Transcriptional activation domains may be fused to both TnsC and Cas7 simultaneously, leading to high-copy recruitment of both Cas7-effectors and TnsC-effectors to the same target site in close proximity, guided by just a single gRNA (FIG. 7H). Nuclear localization signal (NLS) tags are encoded on both proteins as well, but are not shown in the figure. Various combinations of activation domains are possible, leading to distinct levels and intensities of transcriptional activation. Repressive domains may be combinatorically fused to both TnsC and Cas7, to achieve synergistic transcriptional repression. One set of embodiments is shown in FIG. 7I, in which a KRAB domain is fused to one component, and Dnmt3A-3L domains are fused to the other component. Nuclear localization signal (NLS) tags are encoded on both fusion proteins as well, but are not shown in the figure. Other combinatorial repression domains are possible. High signal-to-noise chromosomal locus DNA imaging reagents may be generated by fusing TnsC and Cas7 to distinct fluorescent proteins (FPs), such that a single gRNA leads to targeted recruitment and binding of multiple copies of both TnsC and Cas7 (FIG. 7J). Because this leads to co-localization of two spectrally distinct FPs, background cellular fluorescence can be more readily discriminated against by screening only for foci that show signal from both FPs. In other embodiments, TnsC and Cas7 are instead fused to FPs that can undergo FRET, such that energy transfer and fluorescence at a distinct wavelength leads to high signal-to-noise locus imaging.

The genome perturbation reagents described above may also be generated using Type I-B CRISPR-transposon systems, by fusing effector domains to the same Cas8, Cas7, and/or TnsC subunits described above. This strategy also applies for the combinatorial effector strategies outlined in FIGS. 7H-7J.

The genome perturbation reagents described above may also be generated using Type V-K CRISPR-transposon systems, by fusing effector domains to Cas12k, TniQ, and/or TnsC subunits. This strategy also applies for the combinatorial effector strategies outlined in FIGS. 7H-7J, though there is no equivalent to the multi-copy Cas7 subunit of Cascade in the Type V-K CRISPR-Cas systems; only a single molecule of Cas12k is involved in target DNA binding together with sgRNA.

### Example 5

### Alternative Guide RNA Expression Vectors

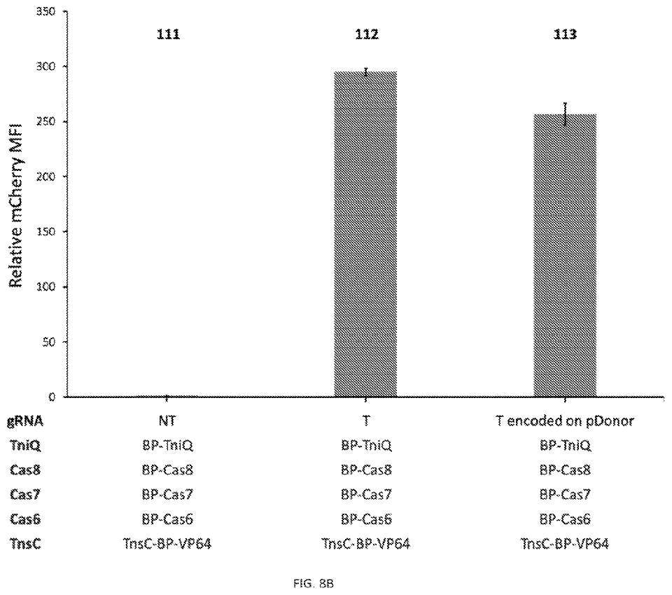

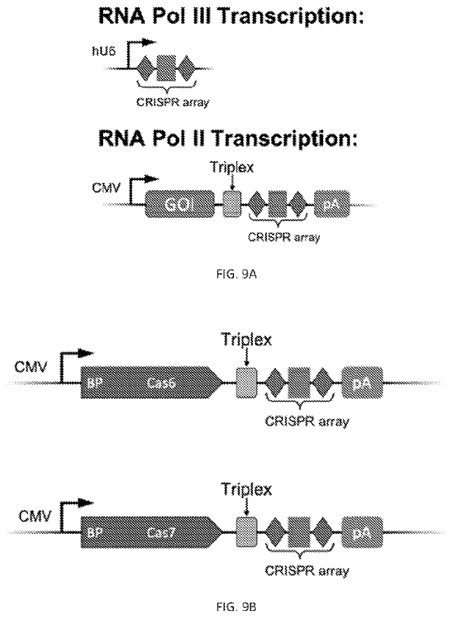

Canonical approaches for exploiting CRISPR-Cas systems for genome editing, including the vast majority of CRISPR-Cas9 methods, encode the guide RNA downstream of an RNA Polymerase III U6 promoter. Within the context of CRISPR-Cas transposon systems such as VchINTEGRATE, expression of the guide RNA on a separate plasmid separate from the mini-transposon donor DNA leads to a risk of self-targeting, as previously described (Vo et al., Nature Biotechnology 39, 480-489 (2021)). Self-targeting could reduce the efficiency of the overall system by inactivating a select pool of expression vectors, and could also lead to undesirable integration events. In order to avoid this, a new donor DNA plasmid (pDonor) was designed that encodes the guide RNA downstream of an RNA Polymerase III U6 promoter immediately adjacent to the mini-transposon donor itself (FIG. 8A). This approach leverages the natural mechanism of target immunity to ‘privilege’ the CRISPR array and prevent self-targeting, leading to proper RNA-guided DNA integration at the intended genomic target site. To determine whether this strategy could be similarly adopted in mammalian cells, gRNA function was tested in the context of transcriptional activation assays relying on TnsC-BP-VP64 fusion proteins. Targeting gRNA encoded on pDonor led to nearly indistinguishable levels of transcriptional activation, as the exact same gRNA encoded on its own plasmid separate from pDonor.

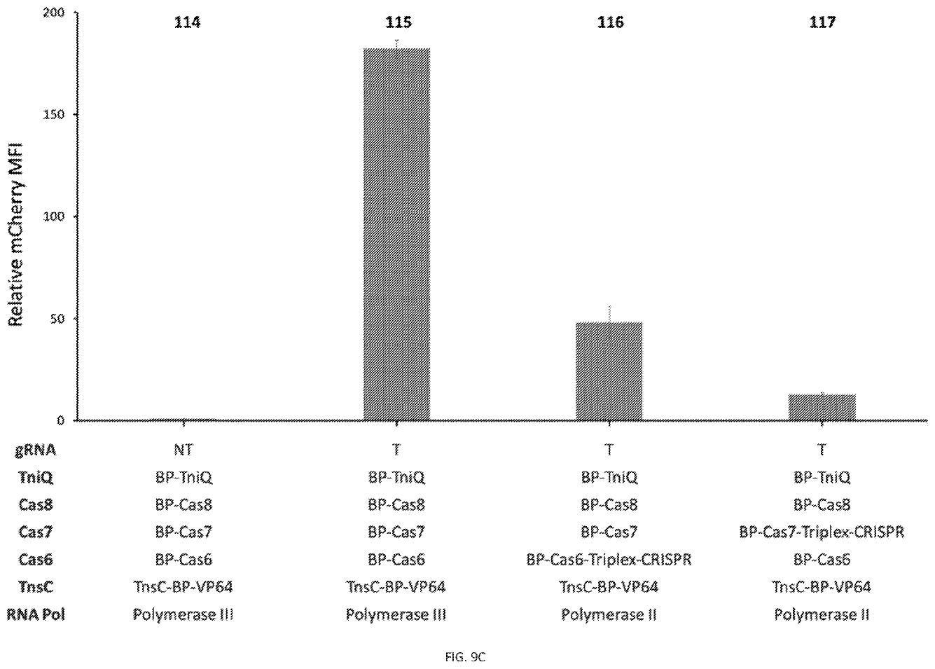

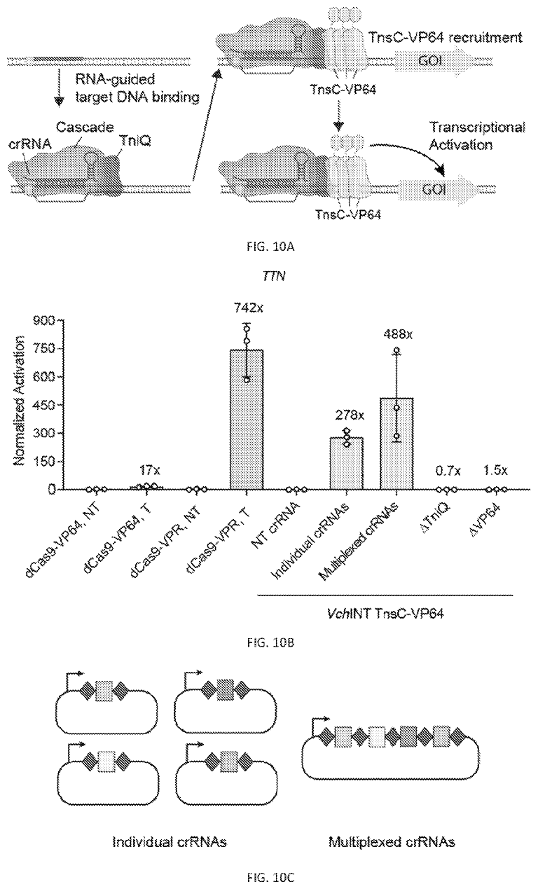

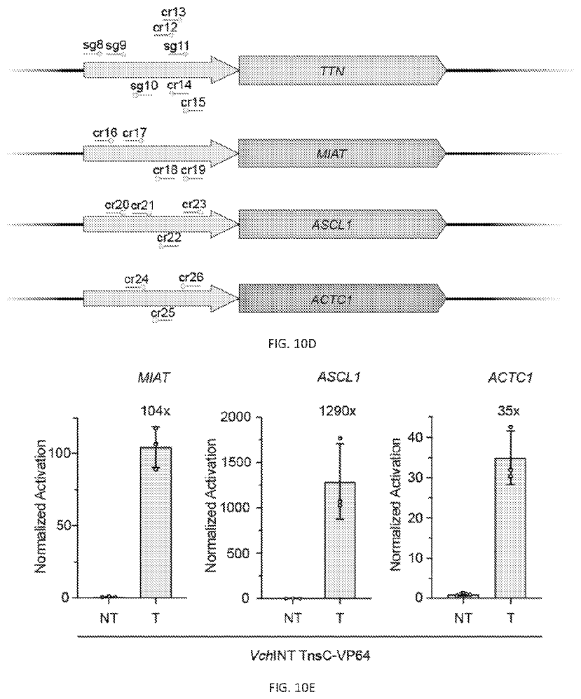

Vectors were designed in which both a VchINTEGRATE protein component and guide RNA were encoded as a type of polycistronic construct on the same RNA molecule, controlled by an RNA Pol II promoter. This strategy reduced the number of separate plasmids required for transfection in order to reconstitute the full INTEGRATE system, and it also promoted cytoplasmic TniQ-Cascade complex formation by exporting the gRNA to the cytoplasm where protein components are initially expressed and localized, prior to nuclear trafficking (FIG. 9A). Cytoplasmic assembly of TniQ-Cascade also obviated the need to place NLS tags on every single protein subunit, since a select few NLS tags on the multi-subunit TniQ-Cascade complex would be sufficient for the entire complex to efficiently traffic to the nucleus. A 110-bp fragment from the MALAT1 locus, previously shown to stabilize mRNA transcripts lacking a PolyA tail (Nissim et al., Mol Cell 54, 698-710 (2014)), was designed and encoded downstream of a gene of interest, in between the stop codon and the CRISPR array. In this context, the CRISPR array was found within the 3′-UTR. Cas6 processing of the pre-crRNA leads to cleavage of the fusion mRNA-crRNA species, but the triplex structure protects the protein-coding mRNA from 3′ exonuclease-based degradation once the poly(A) tag has been severed from the rest of the transcript. Two constructs were designed, in which the MALAT1 triplex sequence and CRISPR array were encoded within the 3′ UTR of either a BP NLS-tagged Cas6 or Cas7, and the ability of these modified gRNA expression cassettes to function for RNA-guided DNA targeting and synthetic transcriptional activation was measured using TnsC-BP-VP64 activators (FIG. 9B). These alternative gRNA expression contexts were functional for transcriptional activation, albeit with slightly reduced efficiency as compared to a separate plasmid encoding the gRNA on a Pol III transcript (FIG. 9C). The CRISPR array may be placed within other 3′-UTRs, such as drug resistance of fluorescence reporter protein genes, and the protein machinery may be further modified in order to optimize the formation of TniQ-Cascade in the cytoplasm.

TABLE 1
Table of plasmids used for the transformation and transfection experiments

| Expt ID | Type of experiment | Plasmid(s) Used |
|---|---|---|
| 54 | Transfection | pSL0302, pSL2552, pSL2554, pSL2549, pSL2661 |
| 55 | Transfection | pSL0302, pSL2552, pSL2554, pSL2549, pSL2556 |
| 56 | Transfection | pSL0302, pSL2552, pSL2554, pSL2620, pSL2621, pSL2622, |
| | | pSL2623, pSL1409 |
| 57 | Transfection | pSL0302, pSL2552, pSL2554, pSL2620, pSL2621, pSL2622, |
| | | pSL2623, pSL2560 |
| 58 | Transfection | pSL0302, pSL2552, pSL2554, pSL2620, pSL2621, pSL2622, |
| | | pSL2623, pSL2578 |
| 59 | Transfection | pSL0302, pSL2552, pSL2554, pSL2620, pSL2621, pSL2622, |
| | | pSL2623, pSL2579 |
| 60 | Transfection | pSL0302, pSL2552, pSL2554, pSL1057, pSL1058, pSL1059, |
| | | pSL1060, pSL2579 |
| 61 | Transfection | pSL0302, pSL2552, pSL2554, pSL2621, pSL2622, pSL2623, |
| | | pSL2579 |
| 62 | Transfection | pSL0302, pSL2552, pSL2554, pSL2620, pSL2622, pSL2623, |
| | | pSL2579 |
| 63 | Transfection | pSL0302, pSL2552, pSL2554, pSL2620, pSL2621, pSL2623, |
| | | pSL2579 |
| 64 | Transfection | pSL0302, pSL2552, pSL2554, pSL2620, pSL2621, pSL2622, |
| | | pSL2579 |
| 65 | Transformation | pSL1567 |
| 66 | Transformation | pSL1969 |
| 67 | Transformation | pSL1970 |
| 68 | Transformation | pSL1971 |
| 69 | Transfection | pSL0302, pSL0341, pSL1057, pSL1058, pSL1060, pSL2081, |
| | | pSL1409 |
| 70 | Transfection | pSL0302, pSL0341, pSL1057, pSL1058, pSL1060, pSL2081, |
| | | pSL2084 |
| 71 | Transfection | pSL0302, pSL0341, pSL1057, pSL1058, pSL1060, pSL2082, |
| | | pSL1409 |
| 72 | Transfection | pSL0302, pSL0341, pSL1057, pSL1058, pSL1060, pSL2082, |
| | | pSL2084 |
| 73 | Transfection | pSL2533, pSL0341, pSL0297, pSL2661 |
| 74 | Transfection | pSL2533, pSL0341, pSL0297, pSL2555 |
| 75 | Transfection | pSL2533, pSL0341, pSL0454, pSL0534 |
| 76 | Transfection | pSL2533, pSL0341, pSL0454, pSL0532 |
| 77 | Transfection | pSL2533, pSL0341, pSL2620, pSL2621, pSL2623, pSL2693, |
| | | pSL1409 |
| 78 | Transfection | pSL2533, pSL0341, pSL1057, pSL1058, pSL1060, pSL2082, |
| | | pSL2084 |
| 79 | Transfection | pSL2533, pSL0341, pSL2620, pSL2621, pSL2623, pSL2082, |
| | | pSL2084 |
| 80 | Transfection | pSL2533, pSL0341, pSL2620, pSL2621, pSL2623, pSL2693, |
| | | pSL2084 |
| 81 | Transfection | pSL2533, pSL0341, pSL2620, pSL2621, pSL2693, pSL2084 |
| 82 | Transfection | pSL2533, pSL0341, pSL2620, pSL2623, pSL2693, pSL2084 |
| 83 | Transfection | pSL2533, pSL0341, pSL2621, pSL2623, pSL2693, pSL2084 |
| 84 | Transfection | pSL2533, pSL0341, pSL0297, pSL2661 |
| 85 | Transfection | pSL2533, pSL0341, pSL0297, pSL2555 |
| 86 | Transfection | pSL2533, pSL0341, pSL0347, pSL2661 |
| 87 | Transfection | pSL2533, pSL0341, pSL0347, pSL2555 |
| 88 | Transfection | pSL2533, pSL0341, pSL0454, pSL0534 |
| 89 | Transfection | pSL2533, pSL0341, pSL0454, pSL0532 |
| 90 | Transfection | pSL2533, pSL0341, pSL2620, pSL2621, pSL2623, pSL2693, |
| | | pSL1409 |
| 91 | Transfection | pSL2533, pSL0341, pSL2620, pSL2621, pSL2623, pSL2693, |
| | | pSL2084 |
| 92 | Transfection | pSL2533, pSL0341, pSL2620, pSL2621, pSL2622, pSL2623, |
| | | pSL2783, pSL1409 |
| 93 | Transfection | pSL2533, pSL0341, pSL2620, pSL2621, pSL2622, pSL2623, |
| | | pSL2783, pSL2084 |
| 94 | Transfection | pSL2533, pSL0341, pSL2620, pSL2621, pSL2622, pSL2623, |
| | | pSL2658, pSL2084 |
| 95 | Transfection | pSL2533, pSL0341, pSL2621, pSL2622, pSL2623, pSL2783, |
| | | pSL2084 |
| 96 | Transfection | pSL2533, pSL0341, pSL2671, pSL2672, pSL2673, pSL2674, |
| | | pSL2783, pSL2084 |
| 97 | Transfection | pSL2533, pSL0341, pSL2620, pSL1058, pSL2622, pSL2623, |
| | | pSL2783, pSL2084 |
| 98 | Transfection | pSL2533, pSL0341, pSL2620, pSL1196, pSL2622, pSL2623, |
| | | pSL2783, pSL2084 |
| 99 | Transfection | pSL2533, pSL0341, pSL2620, pSL2621, pSL2622, pSL2623, |
| | | pSL2783, pSL1409 |
| 100 | Transfection | pSL2533, pSL0341, pSL2620, pSL2621, pSL2622, pSL2623, |
| | | pSL2783, pSL2084 |
| 101 | Transfection | pSL2533, pSL0341, pSL2620, pSL2621, pSL2622, pSL2623, |
| | | pSL2783, pSL2656, pSL2084 |
| 102 | Transfection | pSL2533, pSL0341, pSL2620, pSL2621, pSL2622, pSL2623, |
| | | pSL2783, pSL2644, pSL2084 |
| 103 | Transfection | pSL2533, pSL0341, pSL2620, pSL2621, pSL2622, pSL2623, |
| | | pSL2783, pSL2669, pSL2084 |
| 104 | Transfection | pSL2533, pSL0341, pSL2620, pSL2621, pSL2622, pSL2623, |
| | | pSL2783, pSL1409 |
| 105 | Transfection | pSL2533, pSL0341, pSL2620, pSL2621, pSL1059, pSL2623, |
| | | pSL2783, pSL2084 |
| 106 | Transfection | pSL2533, pSL0341, pSL2620, pSL2621, pSL2622, pSL2623, |
| | | pSL2783, pSL2084 |
| 107 | Transfection | pSL2533, pSL0341, pSL2620, pSL2621, pSL2837, pSL2623, |
| | | pSL2783, pSL2084 |
| 108 | Transfection | pSL2533, pSL0341, pSL2620, pSL2621, pSL2946, pSL2623, |
| | | pSL2783, pSL2084 |
| 109 | Transfection | pSL2533, pSL0341, pSL2620, pSL2621, pSL2947, pSL2623, |
| | | pSL2783, pSL2084 |
| 110 | Transfection | pSL2533, pSL0341, pSL2620, pSL2621, pSL2948, pSL2623, |
| | | pSL2783, pSL2084 |
| 118 | Transfection | pSL2533, pSL0341, pSL0347, pSL2661 |
| 119 | Transfection | pSL2533, pSL0341, pSL0347, pSL2555 |
| 120 | Transfection | pSL2533, pSL0341, pSL2620, pSL2621, pSL2622, pSL2623, |
| | | pSL2783, pSL1409 |
| 121 | Transfection | pSL2533, pSL0341, pSL2620, pSL2621, pSL2622, pSL2623, |
| | | pSL2783, pSL2084 |
| 122 | Transfection | pSL2533, pSL0341, pSL2620, pSL2621, pSL2622, pSL2623, |
| | | pSL2783, pSL2084 |
| 123 | Transfection | pSL2533, pSL0341, pSL2620, pSL2621, pSL2622, pSL2623, |
| | | pSL2783, pSL2084 |
| 124 | Transfection | pSL2533, pSL0341, pSL2620, pSL2621, pSL2622, pSL2623, |
| | | pSL2783, pSL2084 |
| 125 | Transfection | pSL2533, pSL0341, pSL2620, pSL2621, pSL2622, pSL2623, |
| | | pSL2783, pSL2084 |
| 126 | Transfection | pSL2533, pSL0341, pSL2620, pSL2621, pSL2622, pSL2623, |
| | | pSL2783, pSL2084 |
| 127 | Transfection | pSL2533, pSL0341, pSL2620, pSL2621, pSL2622, pSL2623, |
| | | pSL2783, pSL2084 |
| 128 | Transfection | pSL2533, pSL0341, pSL2620, pSL2621, pSL2622, pSL2623, |
| | | pSL2783, pSL2084 |

TABLE 2
Description of plasmids

| Plasmid ID | Plasmid name |
|---|---|
| pSL2533 | p6A_Macrolab CMV_acGFP _noORI |
| pSL0302 | CAGG-eBFP2 |
| pSL0341 | mCherry reporter for CRISPRa |
| pSL0454 | pcDNA3.1 hCO pse_Cascade-Cas7-VP64 |
| pSL0532 | 6A U6-I-E_pse_CRISPR(Hsa07-2) |
| pSL0534 | 6A_hU6_I-E_PseS-6-2_CRISPR(non-targeting) |
| pSL1057 | pcDNA3.1_hCO_Vch_NLS-TniQ |
| pSL1058 | pcDNA3.1_hCO_Vch_NLS-Cas8 |
| pSL1059 | pcDNA3.1_hCO_Vch_NLS-Cas7 |
| pSL1060 | pcDNA3.1_hCO_Vch_NLS-Cas6 |
| pSL1196 | pcDNA3.1_hCO_Vch_NLS-Cas8-T2A |
| pSL1409 | p6A_Vch_hU6_CRISPR(tSL0105) |
| pSL1567 | pCDF_Vch_PT7_CRISPR(Target4)_QCascade_TnsABC_T7Term w/all |
| | Permissive Eukaryotic Terminal Tags |
| pSL1969 | pCDF_Vch_PT7_CRISPR(Target4)_QCascade_TnsABC_T7Term, RPV- |
| | Cas8, w/all Permissive Eukaryotic Terminal Tags |
| pSL1970 | pCDF_Vch_PT7_CRISPR(Target4)_QCascade_TnsABC_T7Term, VP64- |
| | Cas7, w/all Permissive Eukaryotic Terminal Tags |
| pSL1971 | pCDF_Vch_PT7_CRISPR(Target4)_QCascade_TnsABC_T7Term, RPV- |
| | Cas7, w/all Permissive Eukaryotic Terminal Tags |
| pSL2081 | pcDNA3.1_hCO_Vch_NLS-RPV-Cas7 |
| pSL2082 | pcDNA3.1_hCO_Vch_NLS-VP64-Cas7 |
| pSL2084 | p6A_Vch_hU6_CRISPR(SL0264) |
| pSL2560 | p6A_Vch_hU6_CRISPR_ISL0324 |
| pSL2578 | p6A_Vch_hU6_CRISPR (tSL0325) |
| pSL2579 | p6A_Vch_hU6_CRISPR (tSL0325 and tSL0326) |
| pSL2620 | pcDNA3.1_hCO_Vch_BP-NLS-TniQ |
| pSL2621 | pcDNA3.1_hCO_Vch_BP-NLS-Cas8 |
| pSL2622 | pcDNA3.1_hCO_Vch_BP-NLS-Cas7 |
| pSL2623 | pcDNA3.1_hCO_Vch_BP-NLS-Cas6 |
| pSL2644 | pcDNA3.1_hCO_Vch_BP-NLS-TnsB |
| pSL2656 | pcDNA3.1_hCO_Vch_TnsA_BP-NLS |
| pSL2658 | pcDNA3.1_hCO_Vch_TnsC_BP-NLS |
| pSL2669 | pcDNA3.1_hCO_Vch_TnsA_BP-NLS_TnsB |
| pSL2671 | pcDNA3.1_hCO_Vch_BP-NLS-3xFLAG-TniQ |
| pSL2672 | pcDNA3.1_hCO_Vch_BP-NLS-3xFLAG-Cas8 |
| pSL2673 | pcDNA3.1_hCO_Vch_BP-NLS-3xFLAG-Cas7 |
| pSL2674 | pcDNA3.1_hCO_Vch_BP-NLS-3xFLAG-Cas6 |
| pSL2693 | pcDNA3.1_hCO_VP64_Vch_BP-NLS-Cas7 |
| pSL2783 | p6A_hCO_Vch_TnsC_BP-NLS-VP64 |
| pSL2837 | p6AMacrolab_hCO_Vch_BP-NLS-3xFLAG-Cas7-BP-NLS |
| pSL2946 | p6AMacrolab_hCO_Vch_BP-2xNLS-Cas7 |
| pSL2947 | p6AMacrolab_hCO_Vch_BP-3xNLS-Cas7 |
| pSL2948 | p6AMacrolab_hCO_Vch_BP-4xNLS-Cas7 |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A system for effector domain recruitment to a target nucleic acid, comprising:
an engineered Clustered Regularly Interspaced Short Palindromic Repeats (CRISPR)-CRISPR associated (Cas) system or one or more nucleic acids encoding the engineered CRISPR-Cas system, wherein the CRISPR-Cas system comprises:
a) at least one Cas protein; and
b) a guide RNA (gRNA) complementary to at least a portion of the target nucleic acid sequence,
wherein one or more of the at least one Cas protein comprises at least one effector domain.

2. The system of claim 1, further comprising at least one transposon-associated protein, or one or more nucleic acids encoding thereof.

3. The system of claim 2, wherein one or more of the at least one transposon-associated protein comprises at least one effector domain.

4. A system for effector domain recruitment to a target nucleic acid, comprising:
an engineered Clustered Regularly Interspaced Short Palindromic Repeats (CRISPR)-CRISPR associated (Cas) system or one or more nucleic acids encoding the engineered CRISPR-Cas system, wherein the CRISPR-Cas system comprises:
a) at least one Cas protein;
b) at least one transposon-associated protein; and
c) a guide RNA (gRNA) complementary to at least a portion of the target nucleic acid sequence,
wherein one or more of the at least one Cas protein and the at least one transposon-associated protein comprises at least one effector domain.

5. The system of any of claims 1-4, wherein the at least one effector domain comprises a transcription activator, a transcription repressor, a base editor, an epigenetic modifier, a chromosomal locus imaging agent, or a combination thereof.

6. The system of any of claims 1-5, wherein the at least one effector domain is appended to one or more of the at least one Cas protein and the at least one transposon-associated protein at a N-terminus, a C-terminus, or a combination thereof.

7. The system of any of claims 1-6, wherein the at least one Cas protein is derived from a Type-I CRISPR-Cas system.

8. The system of any of claims 1-7, wherein the at least one Cas protein comprises Cas5, Cas6, Cas7, and Cas8.

9. The system of any of claims 1-8, wherein the at least one Cas protein comprises a Cas8-Cas5 fusion protein.

10. The system of claim 8 or claim 9, wherein Cas7 comprises at least one effector domain.

11. The system of any of claims 8-10, wherein Cas8 or the Cas8-Cas5 fusion protein comprises at least one effector domain.

12. The system of any of claims 1-6, wherein the at least one Cas protein is derived from a Type-V CRISPR-Cas system.

13. The system of claim 12, wherein the at least one Cas protein comprises Cas12k.

14. The system of claim 13, wherein Cas12k comprises at least one effector domain.

15. The system of any of claims 2-14, wherein the at least one transposon protein is derived from a Tn7 or Tn7-like transposon system.

16. The system of any of claims 2-15, wherein the at least one transposon-associated protein comprises TniQ.

17. The system of claim 16, wherein TniQ comprises at least one effector domain.

18. The system of any of claims 2-17, wherein the at least one transposon-associated protein further comprises TnsC.

19. The system of claim 18, wherein TnsC comprises at least one effector domain.

20. The system of any of claims 2-19, wherein the at least one transposon associated protein further comprises TnsA, TnsB, or a combination thereof.

21. The system of claim 20, wherein the at least one transposon protein comprises a TnsA-TnsB fusion protein.

22. The system of claim 21, wherein the TnsA-TnsB fusion protein further comprises an amino acid linker between TnsA and TnsB.

23. The system of claim 22, wherein the linker is a flexible linker.

24. The system of claim 22 or 23, wherein the linker comprises at least one glycine-rich region.

25. The system of any of claims 22-24, wherein the linker comprises a NLS sequence.

26. The system of claim 25, wherein the linker comprises a NLS sequence flanked on each end by a glycine rich region.

27. The system of any of claims 1-26, wherein one or more of the at least one Cas protein and the at least one transposon-associated protein comprises a nuclear localization signal (NLS).

28. The system of claim 27, wherein one or more of the at least one Cas protein and the at least one transposon-associated protein comprises two or more NLSs.

29. The system of claim 27 or claim 28, wherein the NLS is appended to the one or more of the at least one Cas protein and the at least one transposon-associated protein at a N-terminus, a C-terminus, or a combination thereof.

30. The system of any of claims 25-29, wherein the NLS is a monopartite sequence.

31. The system of any of claims 25-29, wherein the NLS is a bipartite sequence.

32. The system of claim 31, wherein the NLS comprises a sequence having at least 70% similarity to KRTADGSEFESPKKKRKV (SEQ ID NO: 4).

33. The system of any of claims 1-32, wherein the engineered CRISPR-Cas system is derived from Vibrio cholerae, Photobacterium iliopiscarium, Vibrio parahaemolyticus, Pseudoalterononas sp., Pseudoalterononas ruthenica, Photobacterium ganghwense, Shewanella sp., Vibrio diazotrophicus, Vibrio sp. 16, Vibrio sp. F12, Vibrio splendidus, Aliivibrio wodanis, Aliivibrio sp., Endozoicononas ascidiicola, Parashewanella spongiae or Scytonema hofmannii.

34. The system of any of claims 1-33, wherein the at least one gRNA is a non-naturally occurring gRNA.

35. The system of any of claims 1-34, wherein the at least one gRNA is encoded in a CRISPR RNA (crRNA) array.

36. The system of any of claims 1-35, wherein the gRNA is transcribed under control of an RNA Polymerase II promoter.

37. The system of any of claims 1-36, wherein the target nucleic acid comprises a promoter region.

38. The system of any of claims 1-37, wherein the target nucleic acid comprises an upstream activator sequence.

39. The system of any of claims 1-38, wherein the one or more nucleic acids comprises one or more messenger RNAs, one or more vectors, or a combination thereof.

40. The system of any of claims 1-39, wherein the at least one Cas protein and the gRNA are encoded by different nucleic acids.

41. The system of any of claims 1-39, wherein one or more of the at least one Cas protein and the gRNA are encoded by a single nucleic acid.

42. The system of claim 40 or claim 41, wherein Cas7 is encoded by an individual nucleic acid.

43. The system of any of claims 1-42, wherein a single nucleic acid encodes the gRNA and at least one Cas protein.

44. The system of claim 43, wherein the at least one Cas protein is Cas6 or Cas7.

45. The system of any of claims 2-44, wherein the at least one transposon-associated protein is encoded on a same or different nucleic acid as the at least one Cas protein and the gRNA.

46. The system of claim 45, wherein each of the at least one Cas protein, the at least one transposon-associated protein, and the gRNA are encoded by a single nucleic acid.

47. The system of any of claims 1-46, wherein the system comprises Cas7 or the nucleic acid encoding Cas7 in greater abundance compared to the remaining protein components or nucleic acids encoding thereof.

48. The system of any of claims 1-47, wherein the one or more nucleic acids further comprises a sequence capable of forming a triple helix downstream of the sequence encoding the at least one Cas protein or the sequence encoding the at least one transposon-associated protein.

49. The system of claim 48, wherein the sequence capable of forming a triple helix is in a 3′ untranslated region of the sequence encoding the at least one Cas protein or the sequence encoding the at least one transposon-associated protein.

50. The system of any of claims 1-49, wherein one or more of the at least one Cas protein and the at least one transposon-associated protein comprises a sequence of a ribosome skipping peptide.

51. The system of claim 50, wherein the ribosome skipping peptide comprises a 2A family peptide.

52. The system of any of claims 1-51, wherein each of the at least one Cas protein are part of a single fusion protein.

53. The system of claim 52, wherein each of the at least one Cas protein and one or more of the at least one transposon-associated protein are part of a single fusion protein.

54. The system of any of claims 1-53, wherein one or more of the at least one Cas protein are part of a ribonucleoprotein complex with the gRNA.

55. The system of claim 54, wherein the ribonucleoprotein complex comprises one or more of the at least one transposon-associated protein.

56. A cell comprising the system of any of claims 1-55.

57. The cell of claim 56, wherein the cell is a prokaryotic cell.

58. The cell of claim 56, wherein the cell is a eukaryotic cell.

59. The cell of claim 58, wherein the cell is a mammalian cell.

60. The cell of claim 58 or 59, wherein the cell is a human cell.

61. A composition comprising the system of any of claims 1-55.

62. A method for recruiting one or more effector domains to a target nucleic acid in a cell comprising:
introducing into a cell the system of any of claims 1-55.

63. A method for modulating expression of a target gene in a cell comprising:
introducing into a cell the system of any of claims 1-55.

64. The method of claim 62 or 63, wherein the target nucleic acid comprises the promoter region or the upstream activator sequence of the target gene.

65. The method of any of claims 62-64, wherein the cell is a prokaryotic cell.

66. The method of any of claims 62-64, wherein the cell is a eukaryotic cell.

67. The method of claim 66, wherein the cell is a mammalian cell.

68. The method of claim 66 or 67, wherein the cell is a human cell.

69. The method of any of claims 62-68, wherein the introducing into the cell comprises administering the system to a subject.

70. The method of claim 69, wherein the administering comprises in vivo administration.

71. The method of claim 69, wherein the administering comprises transplantation of ex vivo treated cells comprising the system.
